SEQUENCE LISTING (42 sequences)

SEQ ID NO: 1 - DNA, 2511 bp, Unknown
  Soil Metagenome
atgcttcgcgcggctttggcggcaatggttatggcgggcgctctgacggcgggttcggccgcgggcgagatgttccccttcaccatgccatggaacgacgccggcaccggcaatatcaccgacctgtcggcgtggaacgataagccggcgggggcgagcggcttcgtcaccgtcgcgggcggccatctggtggcgggcggcaaacggctgcaactcctcggcgtcaacgtgaccttcggctccaacgcgcctgagcatgccgatgccgatatcgtggcgcgccggatggcccgcttcggcatcaatatcgtgcggctgcaccatatggacacctatgaggcgccgttcgggattctggaaaaggatcgcgtcaccctcaaccccgactatctcgacaagctggactatttcgtcgccgcgctgaagcggcagggcatttatgtcgacatcaacctgcatgtcggccgcgcctatccgggctttgcgagttggccgggcggcgacagctatttcaagggcgtcgatcacttcgagccgcagatgatccgcttgcagaaggatttcgcccgcgacctgctccaccatcgcaatccctataccggcacccgttatgccgacgaaccggcggtcgccatcgtcgagatcaacaacgagaacggcctgatccgcgaatggggcgccggggcgctcgacgccatgaccgagccgctgcgcggtgagatgacccggcaatggaatgtctggctgaagcagcgctatggcagcgatgcggcactgcgccaagcgtggggcgcgcgcagcgaaccgctcggcaacgagatgttcaccaccggctggcaattgcagacgcttggcggtgcgcacgcaacgctgacgccgaccgcggtcgggctcgccctgaccatgaccggcaaaggccaagagagctggcacacgcagatgcaccaaggcgggctgaatttcaccgccgaacgaccctacaccctgacgctgcggcttcgggccgatcacccgatgaaagtcgcggtgcaggcgatgcagacccacgaaccgtggaaatggttgttgtccgataccatttcggtcggcaccgaatggaagacggtgcatttcacgtttgtgcccgccttcggcgagaccggcgcgcggctgacgctgggcggtctcgggtttgagaccggcacactggagattgccgaagcgagtttgcggcccggcggcacgtcggggctgaaaccgggcgaaaatctcgaccgcggcagcgtcgcgatcagcgaatatagttcccgcttcagccgcacgccggcggcgcaacgcgattggctgaatttcctgtgggataccgaaacccactattgggccgagatgcagcggttcctgaaggccgatctcggcgtcaaatcgctgctcgtcgggacgcagacggtgtacagcccggcgccaatccaatccggcctcgatgtcgtcgacgatcacgcctattggcagcatccgcatttccccggccgcgcctgggatccgggcaactggcggatcaacaatctgccgatggcgggcctcgaaggcggcggcacgattgccgatctggcgctgcgccgcgtgcccggcaagccgttcatcgtgacggaatacaacgcgccagcgccgaacgactaccaaggcgaggccatgccgctggttgcggcgtatggcgcgctacaggattgggacggcattttcctgttcgactacggcggctgggacaacaactggcacaccgatcatatcgacagcttcttcgacagccgctccaatccggtgaaactggcgagcttgatcgcgaccgccgcgatgctgcggcggggcgatgtcgcggcggccgctccgacgcgtgcaagcatgccggaccgcgcggcgtggatcgaagcgctgcgccaatcggcgtatccgcccagcggagccaatttcggcatggcgaaagacgcggccttggcgcgctcggtcggcgcgatcgcgggcaacggggtggcgccgtcatggccggtcaagagcgagacgggtgaactcacttggggtctcggcggcaagaccgtggtgatcgatgcgcctcgcagcaagggtctgatcggcccgcggctcggccatgaatatgacgcgcatggcgtcgggctggaactgaccgaagcgcaaggcgattggggcgtggtgaccgcgaccgtcgttcagggtacggatttttcgtcgccggggcgcattctggtgacgacgctcggccgcgaagagaataccggacagcaatggaccgacgcgacgcggacttcggttggccgcaattggggcgcagcgccggtgctggtcgagggtttgggcgcgcgcatcaccctgcccgttccggcagcgcgggtgtcggcgtttgcgctcgatgcgctgggcaatcgtaccacggcactgccggtgagcggttcgagtcgcgccacaatcgaactcggcgcgcggtatcagacgctgtggtatgaggtggtggtgaagtag

SEQ ID NO: 2 - protein, 836 aa, Unknown
  Synthetic Construct
MLRAALAAMVMAGALTAGSAAGEMFPFTMPWNDAGTGNITDLSAWNDKPAGASGFVTVAGGHLVAGGKRLQLLGVNVTFGSNAPEHADADIVARRMARFGINIVRLHHMDTYEAPFGILEKDRVTLNPDYLDKLDYFVAALKRQGIYVDINLHVGRAYPGFASWPGGDSYFKGVDHFEPQMIRLQKDFARDLLHHRNPYTGTRYADEPAVAIVEINNENGLIREWGAGALDAMTEPLRGEMTRQWNVWLKQRYGSDAALRQAWGARSEPLGNEMFTTGWQLQTLGGAHATLTPTAVGLALTMTGKGQESWHTQMHQGGLNFTAERPYTLTLRLRADHPMKVAVQAMQTHEPWKWLLSDTISVGTEWKTVHFTFVPAFGETGARLTLGGLGFETGTLEIAEASLRPGGTSGLKPGENLDRGSVAISEYSSRFSRTPAAQRDWLNFLWDTETHYWAEMQRFLKADLGVKSLLVGTQTVYSPAPIQSGLDVVDDHAYWQHPHFPGRAWDPGNWRINNLPMAGLEGGGTIADLALRRVPGKPFIVTEYNAPAPNDYQGEAMPLVAAYGALQDWDGIFLFDYGGWDNNWHTDHIDSFFDSRSNPVKLASLIATAAMLRRGDVAAAAPTRASMPDRAAWIEALRQSAYPPSGANFGMAKDAALARSVGAIAGNGVAPSWPVKSETGELTWGLGGKTVVIDAPRSKGLIGPRLGHEYDAHGVGLELTEAQGDWGVVTATVVQGTDFSSPGRILVTTLGREENTGQQWTDATRTSVGRNWGAAPVLVEGLGARITLPVPAARVSAFALDALGNRTTALPVSGSSRATIELGARYQTLWYEVVVK

SEQ ID NO: 3 - DNA, 2586 bp, Unknown
  Wastewater Metagenome
ttgcgtttaaatttcgcctcctttcagttgttagcttggttaatgttttccgtctcagtggatgcattgtccgctgacagtgatttttttccttacaaagtccgatgggacgaagcttctgttagtgcagttaatctgcgcgactggaatcatagacccgctggtaggctcggttgggttacggctcgtgatgggcatctttatgttggaaagtctaggctacgtttctttggcgttaatgttgtctttcggggagcaatgcctgagcgtgatgaggcggagaaaattgcagcacgtttggctaagttaggcttcaatgtggtccgatttcatcatatggatacgcttgttagtcccaatggtctgctcaaggatgatctgcgtacttttgatccagctcagctagataaactggattactttattgcagctttgaagcgagaaggtatctacagcgatcttaatcttcatgtagggcgtctttatcctggttttgatcgttggcgtgatgccgctggaaatcagcagcctgaggcttggaaaggggttgatgttttctatcctcctatggtcgatcagcagaaagagtacgcaaaagagttgctgactcatgtaaatccttatttaggtaagcgatatttagatgagcctgctgtcgccattgtcgagctcaataatgagaatgggctgatctatagttggaggcgcggcgatcttgacctgatgagtgagccgtatcgtggagagttgcaacgtctatggaactcttggcttgttacccactataagagtgacgctggcttgcgtttggcgtgggagacaaaagaagtacctatgggtatggagatgctggtcagtcccggagcacctaccgggcttgttcgtgagtggacgctgcagtctgtggggcaggcgagagcgatacttgatgggtatgaggatgggatccagcatcttaacgttcttcagccaggtactgagcgttggcacgttcaggttcatcaaaaatctttgtcgtttaaggctggcgaactctacaccctacatttgcgattgcgcgctaataagcctcggtctgtacgtctcatggctgtgcaaaaccatgctccgtttcggtctttgtgggagcagaggctcaagcttgattctgagtggcaggagttcgtatttgtttttagttcccctatcgatgaagcgcttgcacggctaactttgggtgatctgggagctgactcaggagagatttggattgcaggcagtagtttaagagcgggcggtgattttaagtttggcgaaagcgactcgttagtgaagcgcaatgttccgatttttactagcagtgactttggaagtcgttcgttgcgcgctcagagagactggctgaactttctgtgggacgttgaggcgcaatattggtcggaaatgcagggttatttaaaaaataagctgggtgtaaagtctttggtcattgggacgcaactaaatcatagtccctcgctgattcagagaaatatggatgtgctggatgcccatgcttattgggatcatccgcgttttccggatggtttatggagccctatcaattggctcattgataataaggctatggccggtgttgatgggggaggggctatttcccgcctcgcattgatgcggctgccgggaaagccatttgttgttacggaatataatcatcctgcacctaatgagtttgcagctgagacttttccgcttgttgctgcatacgcggcgatgcaagactgggatggtgtttttctattcagctatgggactcatagcagaagttggaaaagggattatgttgataattttttcgacataaatgctaatccaaataaatttacgagtactttggcggcggcggcgctttttcgccgtggtgacgtttcttcccaacgaggtgctttttccaccgttctaccaagtcgatccgcatttatagatgctctgcgccagataaataaaaactatcttccgtctgggggggattttggcgttagcagtaattctgcgatgcgcgagcctgttgctttgactgggcctgttcgtgttgattctcttctgccaataaagagcctgacagggcagcttgtctggggtgttgataaaagcccaacagtaagtataaatacgcctttgagtaaaggcttgattggtgccagtcttgctgaaccatttgatgcatctggagtacatttgcaactgttgggttcagatactgggagtggggttgtttttcttacgcttatagatggatcaagtttttctggccccggaagattattaattactgcgcttggtaacagtaaaaataccaatcaagtctgggtggataagacgcgtgcaagccttggggggaagtgggggcaagcccctgttcttgtcgaaggtgttcgtagtcgtattactttgcctatctcaagctctcaagttcgtgcttgggctttagatgaggagggccgccgcaaggagattgttgtggttcgcggaaatgaaaatgcggtaattgaaacgggacctaaatataagagtttgtggtacgaggtggaagttcttgcaaattga

SEQ ID NO: 4 - protein, 861 aa, Unknown
  Synthetic Construct
LRLNFASFQLLAWLMFSVSVDALSADSDFFPYKVRWDEASVSAVNLRDWNHRPAGRLGWVTARDGHLYVGKSRLRFFGVNVVFRGAMPERDEAEKIAARLAKLGFNVVRFHHMDTLVSPNGLLKDDLRTFDPAQLDKLDYFIAALKREGIYSDLNLHVGRLYPGFDRWRDAAGNQQPEAWKGVDVFYPPMVDQQKEYAKELLTHVNPYLGKRYLDEPAVAIVELNNENGLIYSWRRGDLDLMSEPYRGELQRLWNSWLVTHYKSDAGLRLAWETKEVPMGMEMLVSPGAPTGLVREWTLQSVGQARAILDGYEDGIQHLNVLQPGTERWHVQVHQKSLSFKAGELYTLHLRLRANKPRSVRLMAVQNHAPFRSLWEQRLKLDSEWQEFVFVFSSPIDEALARLTLGDLGADSGEIWIAGSSLRAGGDFKFGESDSLVKRNVPIFTSSDFGSRSLRAQRDWLNFLWDVEAQYWSEMQGYLKNKLGVKSLVIGTQLNHSPSLIQRNMDVLDAHAYWDHPRFPDGLWSPINWLIDNKAMAGVDGGGAISRLALMRLPGKPFVVTEYNHPAPNEFAAETFPLVAAYAAMQDWDGVFLFSYGTHSRSWKRDYVDNFFDINANPNKFTSTLAAAALFRRGDVSSQRGAFSTVLPSRSAFIDALRQINKNYLPSGGDFGVSSNSAMREPVALTGPVRVDSLLPIKSLTGQLVWGVDKSPTVSINTPLSKGLIGASLAEPFDASGVHLQLLGSDTGSGVVFLTLIDGSSFSGPGRLLITALGNSKNTNQVWVDKTRASLGGKWGQAPVLVEGVRSRITLPISSSQVRAWALDEEGRRKEIVVVRGNENAVIETGPKYKSLWYEVEVLAN

SEQ ID NO: 5 - DNA, 2916 bp, Lewinella cohaerens
  CDS (1)..(2913)
  sig_peptide (1)..(66)
  mat_peptide (67)..(2913)
atgaccattcatttccgcatacagctatccctatttgcccttgtatttagctcgctcctctttggtcaaaacttctcgaatggctttccttttgcattgcctgtcgatgacaatacctcttctgtatttctaccggcattccctgccagccctataacagaagcgaaaagagtagtacctgagggtaggcaattcgtgcgccaaggtgaagccatccgtttttggggtgtcaatatcacgtcttcagcttgtttccctacccataccgaggcagaaaccatcgctcgacgccttagaaagatggggatcaaccttgtccgttttcatcaccttgacaaccccgcatgggcaggtaatgagggtacaatcttcctcaatagccaagacaataccctccaaattgaccctgtaagcatggatcgcctcaactatttcatttctcgcctcaaacaagaaggcgtctatgtcaatttaaacctccatgttacgcgtacttttcggttaaacgacggtgtccccctcgctgattcaatcgctgactttggtaaagtagtcacgctatacgaccctcagctacaagctctacaaaaagaatatgctaatgaattactggcccaagtcaacccctacactggaataacacttgcactagatcctgtcgtagtaatggtagagatgaacaacgaaaatagcatctacggctggtggaaaagcaacgccttgcgcccttttaaccaaggaggccgactgacggtctatcatcatgaaatgctcaatgaccgttggcataccttcttaggacaagaatatgccgatgatgaaagcctagcagccagttggaacaacggaaccatccccgctggcaccgaggaaaacctcaccaaccccgacctcgaagagggactattacaagccccttggttattggaaacccatgacattgcacaagctaatattactctcgacaataccaatcctcaatcaggtaatcaatgtgttgctttacaggttacacaggcgacgggaacagaatggcatatacagttcaagcaaaacgacctcaacttccaacgcgactctacctatgaattgcggttttgggcacggactgacaccgaacgggatttttccatttccttccttcgtgatgatgccccctatacctggtacagtggtcgaacatttacggccaacactcagtggcaggaattccgcttactgtttacagcttctgaaagtactaccgctggccgactcagtatcagcccacttggtggcaatggcacctattggtttgataacttctcattatctaatccagcggtggatggcctgctaccaggcgaaagcctcaataccgccagcatcaaacgtatcccttggagccaacgcctaagttacacccctgccagggtagctgacctaagccgcttttacgtagctcttcaagcggagcacttccgtgaaatgaaagaatacctcacggatcagttgcttgtgagtgctgctattaccggcaccaatgcgctagttggtcctgccgatgtggtacatcaattggatctcgactatttggatgatcatagctactgggatcatcctcactttcctaatactgcctgggatagctacgactggctcattaacaaccaaccgcaggtgctagaccctaactttgaagccatcacccatgctttttctggactggcacgcaccgatcaacctttcacactttccgaatacaaccacggtgctcctaatcgctaccgcgttgagatgccccatagcatattggcctatgctgcctttcagggggcagatggcatcatgttttatacttacgccggagagcgcaatcaagacaacgatcttgttaataatttctttgacttgcaccgtgaccattccataatggcccaatttccaggggtcgcgatggcttaccgacgagggtaccttcaggaagcacaacaacccttaatggccaattacaaagaagaagatatccataggtttcctatcgtagacaatcaaggccgctgggggcgttatacaccttatgacaaaaggttaattttgacaacaggcgtacagacgggtagttacgacgctccacaaaccagtaattttacggagtggccttctcctccagaagaggtatttacaacattcaatggagaaacaacgctgaacactactgaagggctattaaccaccaataccgataaattttgtagtgttacaggtttcttttctaccgccacagatatgacattggatgccttgaccataaattctggcaatgactttgggactttgcaatggatcagtcttgatgatcaaccactacctgaagctaaaaaatctttaatcaccttaactgctgcccagcaaaacacaaacatgacttggaatggcaccaataccattcacaacaattggggtaacgcaccgacggaacaaaaaccactacaggtggcaatagagatggcattaaatgccgattatataaaactttaccctttagatgtttatgccacacctacggactctatcctagtattaccaaatagtcaaggccattttcctattctcttagatcaataccaatacgaaaccctatggtttggcattaatacttttattggcccagtcagcactcttgaaagcgaccagccaactccatttcacttctaccctaatccggtattaagtggacaacctgtgcaaattacaggaccagaaaaaagcactatactgcttttcaacatcttagggcagttggttaatcaacaagaaatcaatacccctttttatcgcttagataccactggcttaacagctggcacttaccaattagtgttcctaaacgaaaataggcaacgactaaataataccctattgatcatcaaataa

SEQ ID NO: 6 - protein, 971 aa, Lewinella cohaerens
MTIHFRIQLSLFALVFSSLLFGQNFSNGFPFALPVDDNTSSVFLPAFPASPITEAKRVVPEGRQFVRQGEAIRFWGVNITSSACFPTHTEAETIARRLRKMGINLVRFHHLDNPAWAGNEGTIFLNSQDNTLQIDPVSMDRLNYFISRLKQEGVYVNLNLHVTRTFRLNDGVPLADSIADFGKVVTLYDPQLQALQKEYANELLAQVNPYTGITLALDPVVVMVEMNNENSIYGWWKSNALRPFNQGGRLTVYHHEMLNDRWHTFLGQEYADDESLAASWNNGTIPAGTEENLTNPDLEEGLLQAPWLLETHDIAQANITLDNTNPQSGNQCVALQVTQATGTEWHIQFKQNDLNFQRDSTYELRFWARTDTERDFSISFLRDDAPYTWYSGRTFTANTQWQEFRLLFTASESTTAGRLSISPLGGNGTYWFDNFSLSNPAVDGLLPGESLNTASIKRIPWSQRLSYTPARVADLSRFYVALQAEHFREMKEYLTDQLLVSAAITGTNALVGPADVVHQLDLDYLDDHSYWDHPHFPNTAWDSYDWLINNQPQVLDPNFEAITHAFSGLARTDQPFTLSEYNHGAPNRYRVEMPHSILAYAAFQGADGIMFYTYAGERNQDNDLVNNFFDLHRDHSIMAQFPGVAMAYRRGYLQEAQQPLMANYKEEDIHRFPIVDNQGRWGRYTPYDKRLILTTGVQTGSYDAPQTSNFTEWPSPPEEVFTTFNGETTLNTTEGLLTTNTDKFCSVTGFFSTATDMTLDALTINSGNDFGTLQWISLDDQPLPEAKKSLITLTAAQQNTNMTWNGTNTIHNNWGNAPTEQKPLQVAIEMALNADYIKLYPLDVYATPTDSILVLPNSQGHFPILLDQYQYETLWFGINTFIGPVSTLESDQPTPFHFYPNPVLSGQPVQITGPEKSTILLFNILGQLVNQQEINTPFYRLDTTGLTAGTYQLVFLNENRQRLNNTLLIIK

SEQ ID NO: 7 - DNA, 2565 bp, Chthoniobacter flavus
  CDS (1)..(2562)
  sig_peptide (1)..(72)
  mat_peptide (73)..(2562)
atgaatcttcgccaaactctccccgtcctctccgttgcggccgccttacttacggcgccagtcgttcgcgctgccgatttgttccctttcgtcctgccgtgggacgatgccagcccttccatcaccaacgtcagctcctggctggacaagccggccgggaaagacggcttcgtctacacgcacgatggacatcttttcgccggcaagaagcgcatccgtttctttggggtcaacctcgccttcgccggaaatttccccaatcacaacgatgccgaccccgtggcggctcgcatggcaaaatttggcatcaactgtgtccgctttcaccacatggatacgggctttgcgccggccggattgctgaagaaggacaagaagaccttcgacgaggactcgctcgaccggctcgattacttcattgcccagctcaaaaagaacgggatctatgccgacctgaatctccacgtcggcctcgaatatcccggcttcaagaaatgggaaggcgcttcgaacttcttcaagggcgtggacaatttttttccaccgttcatcgagcagcagcgcgagtacgcgcgcatgctgctcacgcacgtcaatgcttatacccacaagccttataccgatgaatcggccgtcgccttcatcgagatcaacaatgagaatgggctgatcatggaatggaacaacgggacgctcgatgccatgcccgatcctttcgccgccgagttgcgaaagcagtggaacgactggttgaaaaagaaatacgacaccccggccaagctcgccggggcatggggaaaaggcgcggagccgctcggtcaggagatgctcaagcccacgcataccgcctggcatctcgagcaacacggcgaggccaagtcggagttgagttccgaacccggcgaagggtccgcgggggaaaccctgcacgtccacgtaacccaaccgggccaggaaagctggcatgtgcagttgggacaggcggggctaaagctggccgccggcaagacctacacgctgcacttgcgcgccaaggcggacgctccccggcggattagcatcgggctgagccaggctcatgaaccgtggaagacacttggcagccagagtgtccggctcaccacggagtggcaggacgtccatttctccataccggtcgcgaataccgaggaaaatgcccgcttctccttcaccagtctcggcagcgcggtcggtgactactggttttccgacgcgtcgctgcgtcctggcggggtcatcgcgctgcaagcgggtgaatcggtcgggaacattcctttcttccgcaagaaagaaatcggcatgcgcaccctcaccgcacaacgcgattggaacgctttcctcgtcgacaccgaggtgaactattggacgggcatgcgccatttcgtccgcgaagagttgcatgcgcacagccaggtcgtcggctcggccaccggcttcagcccatggctggcgcaggcgaagctcgatgtcgtcgatgcgcacagctattggcagcatccgcattttccccacaaaccatgggacccgggcgactggacggtgcaaaacgtctcgatggccggcgcgcccgacggaggcacactcccgggcctcgcactacggcgcgtggccggcaagcctttcatcgtcaccgaatataacgcctccgcaccgaacacctattcgagtgaggctttcctcgaactctgcgccatcgcgggcttgcaggactgggacggggtctttgcttttgcctacagccaccgcgaaaacgattggaacaccgagcacatcatgggctttttcgatatcgaccagcacccgacgaagatggccacgctacccgcggcgctcgcgctctttatgcggggcgatatcaaacccccgggcgaacccgtcatcgcggacaccacctggaacgacgcgcttgaatcggtccgcaaaggaggatcgtgggtcgatgccaatacctacggcattccgaaagaggaagcgtttcggcgcgccatcggcatgcgcatcggccaggccaccaaagtcaacgtgccgccgccatccggagattcgtcggtgattcgcagtgacaacggccagttcacctgggacaccgtttcgcaccgcatgctcctcgcgagtccgcgttcagcgggcgtgatcggctcgctcaaagagggcgaaacgatcgacctcggcagtgtgcgaatcatcccgggtcccacgcggcagaactgggcgacgatcaatgccacggtcatcgccggcccggattttgaacacgcgaagcgcattctcatcacggccacgggtctggccgaaaacaccggcatgaagtggaaggatgcgcagaagtcgagcgtaggcgccgactggggccacgagccttcgctcgtcgaggggatctccgccaagatcggcgtgcctttccagaaaggcgctcaggcgtggtctctcgacgcgcgcgggcagcgccaaaccgagatccctgtgaagcgagggacaggcaaaacggagatcgagatttcccccaatcaacagacgctctggtgggagatcgagatcccgtaa

SEQ ID NO: 8 - protein, 854 aa, Chthoniobacter flavus
MNLRQTLPVLSVAAALLTAPVVRAADLFPFVLPWDDASPSITNVSSWLDKPAGKDGFVYTHDGHLFAGKKRIRFFGVNLAFAGNFPNHNDADPVAARMAKFGINCVRFHHMDTGFAPAGLLKKDKKTFDEDSLDRLDYFIAQLKKNGIYADLNLHVGLEYPGFKKWEGASNFFKGVDNFFPPFIEQQREYARMLLTHVNAYTHKPYTDESAVAFIEINNENGLIMEWNNGTLDAMPDPFAAELRKQWNDWLKKKYDTPAKLAGAWGKGAEPLGQEMLKPTHTAWHLEQHGEAKSELSSEPGEGSAGETLHVHVTQPGQESWHVQLGQAGLKLAAGKTYTLHLRAKADAPRRISIGLSQAHEPWKTLGSQSVRLTTEWQDVHFSIPVANTEENARFSFTSLGSAVGDYWFSDASLRPGGVIALQAGESVGNIPFFRKKEIGMRTLTAQRDWNAFLVDTEVNYWTGMRHFVREELHAHSQVVGSATGFSPWLAQAKLDVVDAHSYWQHPHFPHKPWDPGDWTVQNVSMAGAPDGGTLPGLALRRVAGKPFIVTEYNASAPNTYSSEAFLELCAIAGLQDWDGVFAFAYSHRENDWNTEHIMGFFDIDQHPTKMATLPAALALFMRGDIKPPGEPVIADTTWNDALESVRKGGSWVDANTYGIPKEEAFRRAIGMRIGQATKVNVPPPSGDSSVIRSDNGQFTWDTVSHRMLLASPRSAGVIGSLKEGETIDLGSVRIIPGPTRQNWATINATVIAGPDFEHAKRILITATGLAENTGMKWKDAQKSSVGADWGHEPSLVEGISAKIGVPFQKGAQAWSLDARGQRQTEIPVKRGTGKTEIEISPNQQTLWWEIEIP

SEQ ID NO: 9 - DNA, 2715 bp, Unknown
  Enrichment metagenome
atggtccacccccgcctgcgcagacccctcctccgactgctcctcgccgtttctcccgtcttttccgccctcgctgcgtcggccgcggacggcgtccccttcgacggcttcccctttcacatcccgcccaccggcaccgtccccggcaccgcgcccgccgccctcggcctccccgcccgccccgccgacagcccgatcgtcatccgcggcgaccagttcatccgggccgacaccggcgaacccatccgcttctggggcgtcaacctctccttcgccggcgcgttccccgatcacgaaaacgccgatcgcatcgccgcccgcctggccagcctcggcgtcaacatcgtgcgcttccaccacatcgaccagcgccgcttccccggcggcctctggcaccgcgacgcccccggcgcctccgccaatccccgcgaggacgacatcgagcaccgcgtcttcgaccccgaatccctcgaccgcctcgactacctcgtcgcccgcctgaaggcgcacggcatctacgccaatctcaacctcaaggtctcccgcaccttcagcacctacgacggccccgccttccccgcgcccgccgccgacgaattcaccccccgcaaaggcaaaggcttcgaccagttctacacgccggccatcgaggcccagaaaaccttcgcccgcctcctcctcacccaccgcaacccctacaccggctccacctacgccgccgagcccgccgtcgcccaagtcgagatcaacaacgagaacggcatcctctgggcctggaactacaacctcctcgaccgcctccccgccccctatctcgccgaactctcctcccgctggaacacctggctgcgcgcccgctaccccgacaccgccgccctccgcgccgcctgggcggacggctcggccgccgcatcctccgtcctttcccccggcgcgcccgcgagcaccgatcttctcgccggcgtcgccccggaactccgcaccgccagaagagcccgcgccacgctcctccccccgcccgcgcgcgacaccggcgacgcggacgccgacgacaccaccgcgctgcgcctcaccgtggacgaagtccccgacgccgcctcgtggaacgtccgctgcaactacccgctcaccctctcccccggcgcgacctacctcgccaccctccgcctccgggccaaccgtgaggaaaaaatcgccctgcgcctgcgcgaccccgacaaccaaaacctcgccgcgccgcgcacgctcaacctcgagaccgactggaaacggcacaccctcacctttgccgtccccgccggcgaccaccccgccgacacgctcgccgccctgctctccctcgaagcgggccgccccggtctcgtcctcgacatcgacgccgcctccttccgcctcaacctcctcgccggcctgccctccggccaaggcatcgaccccggcgaccgccccgtcgcctgggtgctccgccgcgacctgcccgaccgcacccccgccaccgtcaccgacatcatgcgcttcctgcgcgacaccgaggtcgcctactggcgcgagatgcacgcctatctccgcgacgaactcggcgtcgtcgcccccatcgccggcaccgccgtcggctactccaccccgcagatccaggccgagaccggcgacttcgtggacacccaccgctactggggcgcgccgcgtttccccaggttcgaccgctccaagccctggaccgtcgaacagaaggccatggtcgcgcatcccggctcatccacattcgaacgcatggccgcccgtcgcgtcttcgggcgtcctttcaccgtcaccgaatacaaccacccgccctccagcgaccatcacgccgaggccttcccgctgctcgccctctacggctccgcgcaggattgggacgcgctcttccaattcgcctacgcccactcccccgacgcctgggaaggcgacacgctccggggcttcttcgacaccgcgcccaaccccgcgcacaccgtcgccgcgctggccgcatccgacatcttccgaaaacgccgcgtcgccccgttctccgaagccgtcgccgtccacgttcctctcgaacgccagctcgaacggcagaacaactacgccttcccgcgactcgtcgaggcctgcgccgtcttcggcggcctccccgccgacgcctggctgcaccgccgcgtcggtctcgcgctccaccccggcgaacaacccgcgtcgctccctcccgccgcctctggccatcaccttgtctgggacgccgcgcacgccggctccgcccacgtccgcttcgtcggcgacggcgccgccggcctcgtcggtttcgtcgccggccggacactcgacctcggctggctgcgtatcactccaggcactacatcgctcgacggtttctccgtcgtcatgctcaacgccgtcgacggccagcccctcggcgcgcccggccgccacctgctcaccgtcgtcgtgcgcgccgccaaccgccacatgggatggaacgccgaccgcaccggcttcggcaccgcgtggggcgaaggccccgccctcgtcgagaccgcccccgtcgacctcgccttcctcaagccggcgcgcgtccacgcgctcgcgcccgacggcacgcgccgcgtcgagctcgcgcccgcggagggttccggctccgccgtccggttccgcgccggcccggaataccgcaccctctggtatgagatttccctctga

SEQ ID NO: 10 - protein, 904 aa, Unknown
  Synthetic Construct
MVHPRLRRPLLRLLLAVSPVFSALAASAADGVPFDGFPFHIPPTGTVPGTAPAALGLPARPADSPIVIRGDQFIRADTGEPIRFWGVNLSFAGAFPDHENADRIAARLASLGVNIVRFHHIDQRRFPGGLWHRDAPGASANPREDDIEHRVFDPESLDRLDYLVARLKAHGIYANLNLKVSRTFSTYDGPAFPAPAADEFTPRKGKGFDQFYTPAIEAQKTFARLLLTHRNPYTGSTYAAEPAVAQVEINNENGILWAWNYNLLDRLPAPYLAELSSRWNTWLRARYPDTAALRAAWADGSAAASSVLSPGAPASTDLLAGVAPELRTARRARATLLPPPARDTGDADADDTTALRLTVDEVPDAASWNVRCNYPLTLSPGATYLATLRLRANREEKIALRLRDPDNQNLAAPRTLNLETDWKRHTLTFAVPAGDHPADTLAALLSLEAGRPGLVLDIDAASFRLNLLAGLPSGQGIDPGDRPVAWVLRRDLPDRTPATVTDIMRFLRDTEVAYWREMHAYLRDELGVVAPIAGTAVGYSTPQIQAETGDFVDTHRYWGAPRFPRFDRSKPWTVEQKAMVAHPGSSTFERMAARRVFGRPFTVTEYNHPPSSDHHAEAFPLLALYGSAQDWDALFQFAYAHSPDAWEGDTLRGFFDTAPNPAHTVAALAASDIFRKRRVAPFSEAVAVHVPLERQLERQNNYAFPRLVEACAVFGGLPADAWLHRRVGLALHPGEQPASLPPAASGHHLVWDAAHAGSAHVRFVGDGAAGLVGFVAGRTLDLGWLRITPGTTSLDGFSVVMLNAVDGQPLGAPGRHLLTVVVRAANRHMGWNADRTGFGTAWGEGPALVETAPVDLAFLKPARVHALAPDGTRRVELAPAEGSGSAVRFRAGPEYRTLWYEISL

SEQ ID NO: 11 - DNA, 2586 bp, Unknown
  Human Stool Microbiome
atgacacgaaaattctttattccactcacgctcggcgggctgttgctcggaagtccgcttacagccgcgaacacggatgccgttccggagggctatcagccgttcccgttgcaatgggatgacacactcgccgggacagcaaccgatgtttctttcctcaacgaaaaacctgccggaaaaaacgggcgtctgatcgttcgcgacgcccattttgtcgaaagttccacaggaaaacgtgtccgtctgattggaatcgggattggcggtgatgcgcttttcgaaatggatcacgctgcggcagaaaaggctgcacggcgtcttgcaaaagccggtgtcaacgtggtccgtttccacaatcttgatggttccgatcgagaccgggatactctcattgatttcaagcaaccgggctccgagcacttcaatcccagacaccttgatattctggactatttcttcgcctgtctgaaaaaggagggaatctatactgtgatgggtctgaaagtcaaccggactctccggaaaggggatgatcttccggaaggcgtcgacaacgccggaaaacgggttgaccgcttcaaccgcgcatggatcgaatcgcagaaacgctgggcgaaaaacctgctgacccgtcaaaatccctatacgaagacaacgctggcggaagatcccgccgttctgagtgtggaactcaacaacgaaagtgcgcttctgtttgaaaatctgaactggatcgacacgcttcccgctccctacaaacgggagctgaccgccctctggaatgacttcctttcccgcaaatacaaaaacgacaaagccctccttgccgcctggaaccgggacaccagtctccccggagcctctctcctgaatccggaaggccgatggggcttcgaacagccggacgcattgaaggttttccgcaataccgccgactcggtttcggcaaccgtaacacggcgcagtaatcaggactggcagatccagtttcagcgttccggactctcccttgaaaacggaaaaacctacacgctggagttcgacgtgcgaggcgaaggcgacccgatccgcatcgttctgtcccaggaccgtcccgactggcacaactgcggactcgaagcctccttatccctgacctccgaatggagacaccagcgctacagcttccaggcaaagaatgcggatcccggacacgtccggatctctttcggtgtcggacatgcacggaaagtggaaatcgccgccgtccgccttttccctggaacgaaaccggtctccgcccgtctctcctccggcagcattccgcttcccgttgcgccgaacgatgccatgagcgctgacttactggagtttatggtcgaacttgacacccgctatgcggaggagatgctggactacctgcggaaggatctcaacgtaaaaagtctggtgatcgacacgcagatcgactggggcggactctccggactccgccgcgaaaaacggatggattacgttgatgcccatgcctactgggggcatccggaattcaccggaggaagctgggagttcaaaccgggttgctggaagattctcaaccagtcacagattccccgcattgtacacggcggctggtgtccgctggagcagttcagccgataccggatcagcaccaagcctttttccatttccgaacacgattatccctatccgcacgactacgcggttgagatgatgccgcttctggtcagcgtggctctgcggcaggactgggacatgcttcagctcttcatccacgggaccttccttacccgcggaaaatccgccggcatcagccacatgtttgatcagaccaatcatcccggcaagatcggtttctttccggctgctgcgctgattttccgtcgcggtatgttcgaacccgctccgaaaacggtggaactccgtcttccggagcagccgtggcgctggttcggcaatcgctttgaccgggcgtgggcggagaccggagtacggcgcagtcttcttgactcccggatgacaatcgttcccgatgcgctgaaaacgcccggtcgggctgaggcatgcgtctccgctcccgatgaaccggaccgcccgatgcgcggatggaccgaaggagagaaaaacttctttacagcggtcgctccgcagtgtattgttctctgcggtcatttcggcggaaggacgatggatgtcggcgatctccgtctccgggcacgtccattccccggcgatttcggcgcggcagttctggtctcccgcgatgaacgtcctcttccggcatccggagatattctcttcacgattgcagggcgcttcgaaaactccggggttatctggaacaaggagcgtgatgcgctcctcaaccgttctccatggtggggcaatcctccggttctcggtacaaaaaccgatgctgccatccgtttcaaaaccgatgggccgcgcacggtctacgcccttgattccaatggaaagcggactgccgaactgccgtccgcatggaaggacgggcatctctccttccatgttcatccgtcacaccgttccatgcactacgaaatcgtcaaaggagaaaacctgtga

SEQ ID NO: 12 - protein, 861 aa, Unknown
  Synthetic Construct
MTRKFFIPLTLGGLLLGSPLTAANTDAVPEGYQPFPLQWDDTLAGTATDVSFLNEKPAGKNGRLIVRDAHFVESSTGKRVRLIGIGIGGDALFEMDHAAAEKAARRLAKAGVNVVRFHNLDGSDRDRDTLIDFKQPGSEHFNPRHLDILDYFFACLKKEGIYTVMGLKVNRTLRKGDDLPEGVDNAGKRVDRFNRAWIESQKRWAKNLLTRQNPYTKTTLAEDPAVLSVELNNESALLFENLNWIDTLPAPYKRELTALWNDFLSRKYKNDKALLAAWNRDTSLPGASLLNPEGRWGFEQPDALKVFRNTADSVSATVTRRSNQDWQIQFQRSGLSLENGKTYTLEFDVRGEGDPIRIVLSQDRPDWHNCGLEASLSLTSEWRHQRYSFQAKNADPGHVRISFGVGHARKVEIAAVRLFPGTKPVSARLSSGSIPLPVAPNDAMSADLLEFMVELDTRYAEEMLDYLRKDLNVKSLVIDTQIDWGGLSGLRREKRMDYVDAHAYWGHPEFTGGSWEFKPGCWKILNQSQIPRIVHGGWCPLEQFSRYRISTKPFSISEHDYPYPHDYAVEMMPLLVSVALRQDWDMLQLFIHGTFLTRGKSAGISHMFDQTNHPGKIGFFPAAALIFRRGMFEPAPKTVELRLPEQPWRWFGNRFDRAWAETGVRRSLLDSRMTIVPDALKTPGRAEACVSAPDEPDRPMRGWTEGEKNFFTAVAPQCIVLCGHFGGRTMDVGDLRLRARPFPGDFGAAVLVSRDERPLPASGDILFTIAGRFENSGVIWNKERDALLNRSPWWGNPPVLGTKTDAAIRFKTDGPRTVYALDSNGKRTAELPSAWKDGHLSFHVHPSHRSMHYEIVKGENL

SEQ ID NO: 13 - DNA, 2712 bp, Fervidibacteria bacterium
atgcggtggtggatgttggtcatcttggcgttcatctctggctttgggggttggtggactatgaggcaagggttatcgcaggaaggcaaacaggatgccttgcttttccctttcgttttaccttgggacgatgcttctccatcggtcaccaacatcagtcactggttgcacaaacctgctggcaagtttgggcatatccgtatcggcgctgacggtcgcctttacgctggcaagcagcgaattcgtttcctgggcgtgaacttgtgtttcggggcatgtttcccgcgcaaagaggactctgaaaaaattgcggcaaggatggcgaagttcggcatcaacattgttcgcttccaccacatggacatgcaagaatttcccaacggcatccgccgtcggggcgtcccccacacccgcgaccttgaccctgaagcccttgacagacttgactacctgattgcccaactgaaacgcaacggcatctatgtcaacttgaacttgctcgtttcccgtcccttcaatgccgccgacggtttgcccaaagaaattgagcaactcggttggaaggagcgacacatcgttggcttcttctacgaaccatgccttgagttgcaaaaggaatatgctcgcaagttgctgacgcaccgcaatccttacacggggctcacctatgctgaagaccctgtcgtcgctttcgtggaaatcaacaacgagaacggtctcattcacgcttggcttagtggctctattgaccggatgccaaaagtttttcaggatgagttgcaacggcagtggcatgcttggctgaaagcccgatacggcacaacggagaagttgcgcaaagcgtggggtgtcaaggaagagcccttgggcaatgaaatgctgaggaacacgaactttgaggcagggttgcaaaattgggttttggagcgacacgcgggtgctgaagcaacagcggaagttgtcgctgaacccatccctgaactcaaaggtcttcgcttcgtccgcatcaatgtcaccaaacgaggacaagcaggctggcatgtccaattccatcaaccgaacctgaaagtccaacccgaccgaccatatacgctttccttctgggcgcgagcggaacgaccttgcaccatttcagtcggaatttcgcaagcccacgaaccttggcagaaccttggcttcagtgccgaagtcaaattgacacaggaatggcgtgaatatcggttcacctttaccctcaacaggggtgacgacaacgctcgcgttatcttcagcaacttgggcgctcaaacgacaacttactggttcgccgccccctcccttcgccctggcggaattgttggcttggcagccaacgaacgccttgaagatggcactgtcccgattttccttcgcgctcgtttcggtgagcgcacacccgaagcgcaacgggactggatgcgatttttgtgggagacggaagatcgctattggcaaacaatctaccgctacctcaaggacgagttggaagttaaggcattggtcatcggcacaatcgtcggatgcagcacacccaacatgatggcaaaacttgactgcgtggacacacacgcttattggcaacatccgatgttcccctcccgaccttgggatcctgaggattggattgtgcccaaccgaacgatggtcaacgaacgaggcgggacgctgccaggtctggcgctgcgacgggtcttaggcaaacctcactccgtcaccgaatacaatcatccagcaccaaacacatactgcagcgaagccttccttttgctggcagcatatgcagccttgcaggattgggatgctatctacgccttcagttactcgcaccgacgagatggttgggacttaaggcgcattcccaacttctttgacattgaccaacatcccacgaagatggttacccttatccctgcagcagcgatgtttgttcggggcgatgtgaagcctgccaagcagcaagtggttgtcaggttgacgaaagagcaagaggttgacttgttgcggcgcagttgggcttggttgctcgtccacgcaggtcatgtcggagttcccaacgaagcagcactcgtccatcgggttgcaattgcgacagatgggaagcgagttccgccaactgctctcaagcccgaacaagtgaaaattggcggcaaccgatttgtgtccgacactggcgaattgatttgggacttgacggaaaaagggcgcggggttgtcatcgttaacgcccaaaacagcaaagccgttatcgggttcgtaggtggcaagcggtttgagttgagcagcgtcgtgattgaacctgggcaaactatgcaagacggttggtgcgccatcacagtcacggcgatggaagggaacttgccaactcgtcccttgcttcgtatccttcgtcccatccgtttgctcatcactgctacaggctacgcagagaacaccgacatgggttggaaagaagttcccggttacccacccaaatcaagttgcggtcgcaactggggcaaaccaccttcgttggtggaaggcatctctgccagcatcaccttgccgttacccgccaagcgagtgcaagcgtgggctttggacgaacggggtcaacggaaatcccaaatcccagtcactgccgacccatcaggcaacgccgtcatccgcatcagtccccaatggcaaacgctttggtatgaagttgaggcgcggtaa

SEQ ID NO: 14 - protein, 903 aa, Fervidibacteria bacterium
MRWWMLVILAFISGFGGWWTMRQGLSQEGKQDALLFPFVLPWDDASPSVTNISHWLHKPAGKFGHIRIGADGRLYAGKQRIRFLGVNLCFGACFPRKEDSEKIAARMAKFGINIVRFHHMDMQEFPNGIRRRGVPHTRDLDPEALDRLDYLIAQLKRNGIYVNLNLLVSRPFNAADGLPKEIEQLGWKERHIVGFFYEPCLELQKEYARKLLTHRNPYTGLTYAEDPVVAFVEINNENGLIHAWLSGSIDRMPKVFQDELQRQWHAWLKARYGTTEKLRKAWGVKEEPLGNEMLRNTNFEAGLQNWVLERHAGAEATAEVVAEPIPELKGLRFVRINVTKRGQAGWHVQFHQPNLKVQPDRPYTLSFWARAERPCTISVGISQAHEPWQNLGFSAEVKLTQEWREYRFTFTLNRGDDNARVIFSNLGAQTTTYWFAAPSLRPGGIVGLAANERLEDGTVPIFLRARFGERTPEAQRDWMRFLWETEDRYWQTIYRYLKDELEVKALVIGTIVGCSTPNMMAKLDCVDTHAYWQHPMFPSRPWDPEDWIVPNRTMVNERGGTLPGLALRRVLGKPHSVTEYNHPAPNTYCSEAFLLLAAYAALQDWDAIYAFSYSHRRDGWDLRRIPNFFDIDQHPTKMVTLIPAAAMFVRGDVKPAKQQVVVRLTKEQEVDLLRRSWAWLLVHAGHVGVPNEAALVHRVAIATDGKRVPPTALKPEQVKIGGNRFVSDTGELIWDLTEKGRGVVIVNAQNSKAVIGFVGGKRFELSSVVIEPGQTMQDGWCAITVTAMEGNLPTRPLLRILRPIRLLITATGYAENTDMGWKEVPGYPPKSSCGRNWGKPPSLVEGISASITLPLPAKRVQAWALDERGQRKSQIPVTADPSGNAVIRISPQWQTLWYEVEAR

SEQ ID NO: 15 - DNA, 2799 bp, Opitutaceae bacterium
atgttccccccgatcgtccgaagcagcttttacctcctccttgccgccgcgctgatcccggcctccgtcaccaccgcgcctgccgcgacgcctgcggatgatctcccttttgccaacggtttcccctttcacatccctcccaccggcaccgtcgccggcacggcgcccgccgccctcgccatccccgctcgtcccgccggagccgacggtcgggtcatcgtccgcggcgaccagttcctccttgccgataccgggacccccatccgtttctggggcgtcaacctctgcttctccggcgctttccccgaccacgccaccgccgaccgcatcgccgcccgtctcgccagtctcggcgtcaacatcgtccgcttccaccacatcgaccagcgccgctttcccggcggcctgtggcatcgcgattcccccggcgccaccaacaacccccgcgaggacaacatcgcgcaccgcacgctcgaccccgaagcgctcgaccgcctcgactacctcgtcgcccgactcaaggaacacggcatctacaccaacctcaacctgaaggtctcccggatcttcagcaccttcgacgatcccgcgttcccggcccccgcgcctggcgaaatccttccgaagaaaggcaaaggcttcgaccagttttacacgcccgccatcgaagcgcagaaagcctacgcccgcctcctcctcacccgtcgcaatgcctggaccggcctgacctgggccgaagaccccgccgtcgcccaggtcgagatcaacaacgaaaacggcatcctctgggcctggaactacaacctgctcgatcgcctgcccgccccctacctcgccgagctcgccgcgcgctggaacacctggctgcgcgcccgttacacggacaccgcggctctccggacagcctgggatccggcgagtggcgcgggcgtctcgcccgcatccgacgtggcacgggcatccctgcccgtgagcgttgcctcccggcccacctccgccgccggtaacgcaaacgccgccgccgcggacgccgacctcctcgccgggatcagcccggctcttttcaccgcgaagaaagcccgggccacgcttgcgcccctgcccgcgcccgacaccggcgatgccgacgccgccgaatccgccggcctgcgcctggccgtcaacaacgtccccggcgacgcaacctggaacgtccgttgcagttactccctcacgcttcccgcgctttcctccggtgcgccctggaccgtcacccttcgccttcgcgccaacaaacccgaaaaaatccgtctgcgcctccgttcccccgagcaaaacaaggatatcgccccgccccgcacgctcaatctcgccaccacctggaagacccacaccgtcacttttgccatccccgagcatgccgctcccctcgccgcgcaactgacgctcgaagccggcctccccggcctcgttctcgacatcgcctccgccagcctccgctcccacacccgttcggggctcccgcgcggcgagggacttgcctccgacgagcgccccgtcacctggattccgcgccgcgacctctccgggcgaaccgatgccgtcgtccgcgatgtcatgcacttcctgcgcgacaccgagatcgcctactggcgtgaaatgcacgcgttccttcgtgatgaactccgcgtcgccgctcccatcacgaccacggccgtcggttacaccacgtcgcagatcgccgccgaaaccgctgacttcatcgacacccatcgctactggggcgcgcctcgctttcccggtttcaaccgcagcaaaccctggaccgtcgaacaaaaagccatggtcgcccgtcccggcgaatccgccatcgaacgcatggccgcccgccgcgtcttcggcctccctttcaccgtcaccgaatacaaccacccgccctccagcgatcaccacgccgaaggcttccccctcctcgccctctggggcgctgcgcaggattggaacggcctcttcgaattcgcctactcccacagcgacgcctgggaaagcgacaccatgaccggcttcttcgacaccgctcccaaccctgttcacaccgtcgccgctctcgccgcctccgacctcttccgcaaccgccgcctcgcccccctcgctcccgcgaaatccggatacgtgcccctcgatcgccagctcgaacggcaaaacaacgacaccttccctcgcctcatcgaggccgacgccgttttcggcggactcccgcccgacgcctggctcgccaatcgtgtcggcctcgttcgccgcgccgatgaacagccggaaacgctcccgccgccgccggccagccagcaactcgcctggaccgccaccgatcccgccaccgcccacgtccgctacacaggcgagggcgtcgccggcctcgtcggcttcgtttccggccagaccctcgatctcggctggctccgtatcacccccggagatacatcgctcggcggtttttccgtcgtcatgctcaactccgtggacggtcagcccctcggcgcatccggccgctatctgctcaccacggcggtgcgcgccgccaaccgcggcatgggctggaacgccgaccgcaccggcttcgacaaaaaatggggatccggccccgcccaggccgaatccgctccggtcacgctcgacttcgcctccgcctccggcgtccgtgtgtatccgctgaatcccgacggaacacgccgcccggaactcccgcccgcctccaccccgggccgcttcgaagccacacccgccagcaaaaccctctggtttgaaatcacattcccgtaa

SEQ ID NO: 16 - protein, 932 aa, Opitutaceae bacterium
MFPPIVRSSFYLLLAAALIPASVTTAPAATPADDLPFANGFPFHIPPTGTVAGTAPAALAIPARPAGADGRVIVRGDQFLLADTGTPIRFWGVNLCFSGAFPDHATADRIAARLASLGVNIVRFHHIDQRRFPGGLWHRDSPGATNNPREDNIAHRTLDPEALDRLDYLVARLKEHGIYTNLNLKVSRIFSTFDDPAFPAPAPGEILPKKGKGFDQFYTPAIEAQKAYARLLLTRRNAWTGLTWAEDPAVAQVEINNENGILWAWNYNLLDRLPAPYLAELAARWNTWLRARYTDTAALRTAWDPASGAGVSPASDVARASLPVSVASRPTSAAGNANAAAADADLLAGISPALFTAKKARATLAPLPAPDTGDADAAESAGLRLAVNNVPGDATWNVRCSYSLTLPALSSGAPWTVTLRLRANKPEKIRLRLRSPEQNKDIAPPRTLNLATTWKTHTVTFAIPEHAAPLAAQLTLEAGLPGLVLDIASASLRSHTRSGLPRGEGLASDERPVTWIPRRDLSGRTDAVVRDVMHFLRDTEIAYWREMHAFLRDELRVAAPITTTAVGYTTSQIAAETADFIDTHRYWGAPRFPGFNRSKPWTVEQKAMVARPGESAIERMAARRVFGLPFTVTEYNHPPSSDHHAEGFPLLALWGAAQDWNGLFEFAYSHSDAWESDTMTGFFDTAPNPVHTVAALAASDLFRNRRLAPLAPAKSGYVPLDRQLERQNNDTFPRLIEADAVFGGLPPDAWLANRVGLVRRADEQPETLPPPPASQQLAWTATDPATAHVRYTGEGVAGLVGFVSGQTLDLGWLRITPGDTSLGGFSVVMLNSVDGQPLGASGRYLLTTAVRAANRGMGWNADRTGFDKKWGSGPAQAESAPVTLDFASASGVRVYPLNPDGTRRPELPPASTPGRFEATPASKTLWFEITFP

SEQ ID NO: 17 - DNA, 2763 bp, Opitutaceae bacterium
atgatcgcccatcgccccctgtttctcgtctgtcgcctcctgttcagcggactggttgcctgttgcatttccccaaacctcctgaacgccgcctccccgcccggcctctttcccttcaccatcgcctgggactctccgcccaccgccgtcacggacgccagcgtcgctgatctcaccggctggctcgacgcccccgcaggcaaacacggctacatccgcgccgaaggctccgacttcatcaccaccgacaccggccaacgcatccgttttctcggcgtcaacctcgcctttgccgccaatttccccgagcacgccgacgccgaaaaactcgccgcccgcctcgcccgcctcggcgtcaactgcgtccgcttccaccacatggacaggaccggtctcctgcgcaacgccgccggtcgccgctccagcatctggagcgaaaacgccgacggccagaccctcgatcccgcgcaactcgaccgcctcgactacctcttcgcccaactcaaggcgcgcggcatttacgccaacatcaacctccacgtctcccgcacctaccccggtttccccgccggctccagataccacaaggcgctcgacatgtttgtccccggcatgatcgccctccagaaacaatacgcccgcgacctcctgcatcacaaaaacaactacaccgggctccgctacgccgacgaccccgccgtggccatcgtcgaaatcaacaacgaaaacggagtcgttggacgctggtggcgcggcaacctcgacacgctcgatccgctttacgttggcgaactcaacacccgctggaacgcttggctcacccgcaaccacggctcacctgccgccgcgctcgtcgcctggaaaaacgccgccgccgccgattccacgcccaccggccccgaactcctcaaaaatcaccgtctcgccaatctcgccaaaaactggacgctccaaaactcctccccctcccccctgctcgccctcgaaccgcccgccagccccggccccggcatcgaaccgcaaggcatcgtcctgcgcgtcctccccgacgctgccaaaaactcccgcgcctcccttttgcaacccgtctccctcaagcccggcgcgcgccacaccttgcgcatcaccctcgaggccgattctcccgccgaactcatcctcgacatcaaggacgcgcgtccgccctggcgcacccacctctccataaaaattcccgccaccgacacccttcgtaccgtcgaaaacaccttcgtctataaggaaaacgccccggcctccggcatccgcctcgccctcaacctccgcgccaccgccagccccgcctcgaacaatttccgcgttcaggaaatctcgctccgcgaaggcggctcctccgcgaaactccccgccttcgttgacgcggaaaacgccaacaattccgccaacgccgccgcttccgccgccgctcccacttcggcgcaccgcgccctcgcctacctccgtcgcgacggctttgaaaccttctcgcccgccgcctgcgacgactggctccgcttcctctgggaaaccgaggacgactactggaccgacatgcgcgaatacctccgccgcgacctcggcatccgctccatactcgtcggctcacaactcggcgcctacagcctgctccccctccagcagaaattcgacgcccttgaccaccacgcctactggcagcacccgcaaacccaggacaacgggcgcaggatcgtccaaaacctctccatggtcaacgagcccggcggagcctacgccgcctcgcccgcgttttaccgcgctgccggaaagccgtatctgctcaccgaatacaatcactcgtcgcccaacacctttggcgcggaggccttccccatcatctccgcctacggcgcgttccaggactggagcggcatcttcgtttactcttacgcgcacggcaccgcgccgtgggacgccggataccagcgcggccagttcgacattgaccagcatcccctcaagcttgcgacccttcccattgccgccgcccttttcctgcgtggcgacaccgacgcccctgcgggtgtcaccaccaaaaccaccaccatcactccggaggaatacctgcgccagatgcgccgtgtcggccccaacgtttcggcgatcaccgccggagccacccgcaccgacgcccttcgccaccgcgtcgccttcgccctcgccgaaaacaaaagtggcgcgggcgtcccgcccgcaaccgttgccactccgtctgccggacccatcaccaccgagggcggcgcactcacttgggacgccgcgcccaacgcgggcattttcaccattcgctcacctcgcaccaaggcagccatcggcttcgcctgcggacgctcctttgacctcgacggcctcgtcattacgcccggccaaaccctccaggactggtcaaccattgccgtctcccaaatgagcggcgaccgcgtcggctctcccggacgcgccctgctcgtcgcctgcggctacatcgaaaacaccgggcaaatctggcacgaccccgtagccaaaaacagcgtcaaggaatcgggtcgcgccccaacgctcgtcgagggcattcccgcgaaaatcaccctcactcccgcccccggcgtcaccgccgtcgaagtctggtcgctcgacgaacacggacgccgcgcccgctccgtccccgtcacccgcgatggccccgccgccacctttcacatcggacccgattaccggaccatctggtatgaagtcatcactcaataa

SEQ ID NO: 18 - protein, 920 aa, Opitutaceae bacterium
MIAHRPLFLVCRLLFSGLVACCISPNLLNAASPPGLFPFTIAWDSPPTAVTDASVADLTGWLDAPAGKHGYIRAEGSDFITTDTGQRIRFLGVNLAFAANFPEHADAEKLAARLARLGVNCVRFHHMDRTGLLRNAAGRRSSIWSENADGQTLDPAQLDRLDYLFAQLKARGIYANINLHVSRTYPGFPAGSRYHKALDMFVPGMIALQKQYARDLLHHKNNYTGLRYADDPAVAIVEINNENGVVGRWWRGNLDTLDPLYVGELNTRWNAWLTRNHGSPAAALVAWKNAAAADSTPTGPELLKNHRLANLAKNWTLQNSSPSPLLALEPPASPGPGIEPQGIVLRVLPDAAKNSRASLLQPVSLKPGARHTLRITLEADSPAELILDIKDARPPWRTHLSIKIPATDTLRTVENTFVYKENAPASGIRLALNLRATASPASNNFRVQEISLREGGSSAKLPAFVDAENANNSANAAASAAAPTSAHRALAYLRRDGFETFSPAACDDWLRFLWETEDDYWTDMREYLRRDLGIRSILVGSQLGAYSLLPLQQKFDALDHHAYWQHPQTQDNGRRIVQNLSMVNEPGGAYAASPAFYRAAGKPYLLTEYNHSSPNTFGAEAFPIISAYGAFQDWSGIFVYSYAHGTAPWDAGYQRGQFDIDQHPLKLATLPIAAALFLRGDTDAPAGVTTKTTTITPEEYLRQMRRVGPNVSAITAGATRTDALRHRVAFALAENKSGAGVPPATVATPSAGPITTEGGALTWDAAPNAGIFTIRSPRTKAAIGFACGRSFDLDGLVITPGQTLQDWSTIAVSQMSGDRVGSPGRALLVACGYIENTGQIWHDPVAKNSVKESGRAPTLVEGIPAKITLTPAPGVTAVEVWSLDEHGRRARSVPVTRDGPAATFHIGPDYRTIWYEVITQ

SEQ ID NO: 19 - DNA, 2535 bp, Paludibacterium yongneupense
atgacgctacgtttgcgcgtgctgctatgcctgtttgcagcctgcggcgcgcatgccgccgacgcaccgctgttcccctatcatttgccctgggatgacgcttcgctgaacctcagtaatctgcatgcctggaatcctgcgcctgccgggcgttccggttttgtcggcgtccgcaacgggcatctgtacgccgagggccgccgcctgcgcttgctgggggtcaactgcgtattcggcggcgcgctgcccgagcatgacgtcgccgagcgcatcgccgcgcgcatggcccgcttcggcatcaatgccgtccgcttccatcatatggacacccggccggctccggatggcctgctgctggcggaccgcctgaccttggatccgaaagcactcgaccgtctcgactacttcatcgcggccctgaaacgggtgggcatttacagcgatctgaatctgcatgtcggtcgcagctatccggggttcgcaccctggagcgacgcgcaaggcaggccgcagcctcagtactggaagggcgtcgacctgttctacccgcccatgatcgcgatgcagcgcgattatgcccgcaccctgctgtcgcatcgcaacccgtataccgggcatcaataccgcgacgagcccgcgctggcgctggtcgagatcaataatgaggacggtttgatacgggagtggcaggatggggcgcttgaccgtatgagcgagccctatcgttccgaattaagacagcgctggaatcgctggctgtcggcgcattaccggtcgaccgccgagctggcccgcgcctggagcgcacgtgacgacgtgcagggagacgagcgcttcgatacgggtggcaatggctggaatctgcaggtggtggagccggcgcgtgcgcgccagcagacggagggcgacggctcgctgcgggtgacggtcgaggcgacagacagcgagaactggcatgtgcagctgcaccagctgcggcaaagcttcgcggccgggcaaccctacaccttgcgactgcggctgcgtgccgaccgtccgctgcgggtgcggctggcggcgatgcaggcgcacccgccctggcagtcgctgtggcaaacggcggtggatgtcgacacccgatggcgcgactatcgttttacctttgcccccgtcgctggggatggtgtggcgcgttttacgctgggcggcctcggggaacagcgctccaccctgtggttgggcatggcgagcctgaagacaggagggcgcctgggactgctggctggcgagtcgctcgaggggagcggagtcgacatctttgcgcgtggcgacgagggcgcccgcagcgcgcaggggcagcgcgactggttacagtttctatgggacacagaaacagaatactgggatggcatgcgtaattttctgcgccagaccgtgggcctgcgttccttgctgattggaacgcaggtcgggtatagcccggccccgatccaggcgcggatggatgtcgtggatggccatgcttactggcagcatccgcgctttcccggccggccgtgggatcccgcagactggagtgtggccaatacgcccatggcgggcatcgatggcggcggcaccctggccgacctggctctgcgtcgcatggccggcaagccttttgtcgtcaccgagtacaatcatcccgcgccgggtgagttcgtcgccgagggactgccgcttctggccgcctatgccgccctgcaggactgggatggcgtatttgttttcgattatggtgccggcgatcacgatccgggtttcattgcttcgtatttcgacattcaggccgatcccggcaagatgagtgcgctgccggccgcggccgccttgttccggcgcggtgacgttgcgacgcccgggagcgcgggcggcgccttaccttcgcccgcggccatgatcgaggccatgcgtgccgggaataccatgccggcggcggatcagtttggcaccgcgcgcaacgaagcgctgaggcggccggtcgcgctcggcagcacgcctgccggtccgcggccgttaccggtgcgtgcgatcggcgggcaattggtatggggcgaggacggccggaagacggtcgtgatcgacactccgcgcagcaagggattggtcggtgccggattgggacgggacgtcgatgccggcggcgtgggcttgcgtctgctgcaggcccgccacaatagcggcgtgctgctggccacactgatggacgggcgtgacttttcgcacccgggccgggttctgctgacggcgatcggaagcgaggaaaacagcgggcagcgctggctggacgaggcgcacaccacgctcggacggcaatgggggcaggcgccggtatgggtcgagggtattgccgcccgcatcgtattgccggtcgcggcggcgcgggtcggcgcctgggcgctcgacgagcgcggacaacggcggcaggccctgccggtggctggcgaccacgcgcatgcggtattcgaaaccagccccggctaccgcgctctttggtatgagcttgaaatccgctga

SEQ ID NO: 20 - protein, 844 aa, Paludibacterium yongneupense
MTLRLRVLLCLFAACGAHAADAPLFPYHLPWDDASLNLSNLHAWNPAPAGRSGFVGVRNGHLYAEGRRLRLLGVNCVFGGALPEHDVAERIAARMARFGINAVRFHHMDTRPAPDGLLLADRLTLDPKALDRLDYFIAALKRVGIYSDLNLHVGRSYPGFAPWSDAQGRPQPQYWKGVDLFYPPMIAMQRDYARTLLSHRNPYTGHQYRDEPALALVEINNEDGLIREWQDGALDRMSEPYRSELRQRWNRWLSAHYRSTAELARAWSARDDVQGDERFDTGGNGWNLQVVEPARARQQTEGDGSLRVTVEATDSENWHVQLHQLRQSFAAGQPYTLRLRLRADRPLRVRLAAMQAHPPWQSLWQTAVDVDTRWRDYRFTFAPVAGDGVARFTLGGLGEQRSTLWLGMASLKTGGRLGLLAGESLEGSGVDIFARGDEGARSAQGQRDWLQFLWDTETEYWDGMRNFLRQTVGLRSLLIGTQVGYSPAPIQARMDVVDGHAYWQHPRFPGRPWDPADWSVANTPMAGIDGGGTLADLALRRMAGKPFVVTEYNHPAPGEFVAEGLPLLAAYAALQDWDGVFVFDYGAGDHDPGFIASYFDIQADPGKMSALPAAAALFRRGDVATPGSAGGALPSPAAMIEAMRAGNTMPAADQFGTARNEALRRPVALGSTPAGPRPLPVRAIGGQLVWGEDGRKTVVIDTPRSKGLVGAGLGRDVDAGGVGLRLLQARHNSGVLLATLMDGRDFSHPGRVLLTAIGSEENSGQRWLDEAHTTLGRQWGQAPVWVEGIAARIVLPVAAARVGAWALDERGQRRQALPVAGDHAHAVFETSPGYRALWYELEIR

SEQ ID NO: 21 - DNA, 2550 bp, Dickeya species
atgatgcgaatccgtagggtagtactggcgctggcgttgtctctgaccttccccgctatcgccggtgagctgtttccctttactttgccgctggatgtccgctccgacggcagccttaccgacctttcgtcatggaacgacaaaccggcaggcgcacgcgggtttgtgacggtcgacggcagccacctgacagttggcggcaaacggctgcgattcctcggcgttaatatcgtctttggttcgaccgcgccgacacatgccgacacagacgctatcgcccgccgcctggcacgcttcggcatcaatctggtgcgttttcatctgatggacgcccgccctgcgcccgacggcatcctgcagcgcgatctgcgcacccttgacccagacaccctcgaccgcgtcgattattttatctcgacgctggcgcgagaaggcatctacgccgatctcaacttgcacgtcggccgacaatatcccggcatgggagagagttgggcggatgggcccaaatactggaaaggcgtcgacctgttttatccgccgatgtttgtacagcagcaggagtatgcccgcgccctgctgacgcaccgcaatccttacaccggtcatcgctatactgaagaacccgccgtggcgttcgtcgagatcaataacgaagacggattgatccgggaatggcggtctggctcgctcgacatgatgcccgcgccgtttcgcactgagctcgcccgccaatggcgtgtctggctcggacatcgttatgctggtgacgacgcgttacgcgcgggctggggcgtgcgggaagagccttacggcgcggaaatgctgtcagagcggatcgggtcgaaaagcagcgacccaggctgggtgctgcaaacccttggcggcgccaacgcgacgctcgccacaacagattccggactgatgctgtcgatgacgacaccggggcagctcggctggcatacgcagttgcactacaatcacctctcgttccagacccgccacgcctatacattgtcgctccggttacgtgccgatcacccgctgacgctgtccgtacaggcgatgcaatcgcacgccccctggcaaacgttgtggtcgcagaaaatcccggtcggcaccgactggcaggatgtcacggtgacatttatgccgcagcaggacgacgcgacggcacggctcacactgggtggcctcgggctggataccggtcgtctggatatcgcccatgcgcggcttcgtaccggcgggttgctcggactacaacccggcgaaagcctcacgcacggcacgctcgatatcattccgtttgcctcccggttgaaccgaaccgtcgcggcacagcgcgactggctgagttttctgtgggacaccgaagcgcagtattggcgaagcatgcgggactttctgaaaggtgaactgggcgtacgctcgccagtcatcggaacgcaggtctcctacagcccggcggccatccagcagacgctggacgtggttgacggccacgcctactggcagcacccgcgttttccgggcaaaccctgggatcgcaacaactggtttatcggcaactcgccaatggccggtattcccggcggcggcacgcttgccgacctagcattgcatcgtgtccccggcaagccgtttatcgtcagcgagtacaaccatcctgcgcccagcctctggcaaggcgaggcgatgccgctggccgccgcctatggcgccctgcaggactgggatggcatcgccgtttacaactatggcgccagtaagcgtaactggcaggccgactttatcaccgactacttcgacagcgtcgctaacccggtcaagatgaccagtctggtggctgccgccgcgctgctgcggcgtgaggatgtgcgcgccgacaagccgcaaccacagccgatgccggatcgcaccgtctttatcgacgcgctgcgtcagggcggccgcctgccgggtgcggacagcctgggcgcgccacgtgacgccgcgctggcgggatgggtcagcatcgccacgccagcgggcgaaccgccgtcatggccggtacgtagccgcaccgggcaactgatctggggtgtggacggcatcggcggcaaaaccgtggtggtcgatacgccgcgcagtaaagggcttatcggcgcccggctcggtcaggtttatgacgcgcacggcgtcgggctggaagtgacggcggcgcgcaacgactggggagtcctgctggcgaccgtactggacgggcagagtttcaccgcgccgagccgcattctgctgacgacgctggggcaggaggaaaacaccggccagcgctggcttgatgccgcaaaaaccaccattggcagccatttcggcaccggcccggtgctggtcgaaggcatcggcgcccgcatcaccctgccggttgcgccgtcccgggtcagcgcctgggcgcttgacgcgcgcggccagcggcagacgccgataccggtcggcggcacgcaacatgccacactggacgtcgatgagcgctatcgtactctgtggtatgaaatcgacattcgataa

SEQ ID NO: 22 - protein, 849 aa, Dickeya species
MMRIRRVVLALALSLTFPAIAGELFPFTLPLDVRSDGSLTDLSSWNDKPAGARGFVTVDGSHLTVGGKRLRFLGVNIVFGSTAPTHADTDAIARRLARFGINLVRFHLMDARPAPDGILQRDLRTLDPDTLDRVDYFISTLAREGIYADLNLHVGRQYPGMGESWADGPKYWKGVDLFYPPMFVQQQEYARALLTHRNPYTGHRYTEEPAVAFVEINNEDGLIREWRSGSLDMMPAPFRTELARQWRVWLGHRYAGDDALRAGWGVREEPYGAEMLSERIGSKSSDPGWVLQTLGGANATLATTDSGLMLSMTTPGQLGWHTQLHYNHLSFQTRHAYTLSLRLRADHPLTLSVQAMQSHAPWQTLWSQKIPVGTDWQDVTVTFMPQQDDATARLTLGGLGLDTGRLDIAHARLRTGGLLGLQPGESLTHGTLDIIPFASRLNRTVAAQRDWLSFLWDTEAQYWRSMRDFLKGELGVRSPVIGTQVSYSPAAIQQTLDVVDGHAYWQHPRFPGKPWDRNNWFIGNSPMAGIPGGGTLADLALHRVPGKPFIVSEYNHPAPSLWQGEAMPLAAAYGALQDWDGIAVYNYGASKRNWQADFITDYFDSVANPVKMTSLVAAAALLRREDVRADKPQPQPMPDRTVFIDALRQGGRLPGADSLGAPRDAALAGWVSIATPAGEPPSWPVRSRTGQLIWGVDGIGGKTVVVDTPRSKGLIGARLGQVYDAHGVGLEVTAARNDWGVLLATVLDGQSFTAPSRILLTTLGQEENTGQRWLDAAKTTIGSHFGTGPVLVEGIGARITLPVAPSRVSAWALDARGQRQTPIPVGGTQHATLDVDERYRTLWYEIDIR

SEQ ID NO: 23 - DNA, 2712 bp, Fervidibacteria bacterium
atgcggtggtggatgttggtcatcttggcgttcatctctggctttgggggttggtggactatgaggcaagggttatcgcaggaaggcaaacaggatgccttgcttttccctttcgttttaccttgggacgatgcttctccatcggtcaccaacatcagtcactggttgcacaaacctgcgggcaagtttgggcacatccgtatcggcgctgacggtcgcctttacgctggcaagcagcgaattcgtttcctgggcgtgaacttgtgtttcggggcatgtttcccgcgcaaagaggactctgaaaaaattgcggcaaggatggcgaagttcggcatcaacattgttcgcttccaccacatggacatgcaagaatttcccaatggcatccgccgtcggggcgtcccccacacccgcgaccttgacccagaagcccttgacagacttgactacctgattgcccaactgaaacgcaacggcatctatgtcaacttgaacttgctcgtttctcgccccttcaatgctgccgacggtttgcccaaagaaattgagcaactcggatggaaggagcgacacatcgttggcttcttctacgagccatgtcttgagttgcaaaaggaatatgctcgcaagttgctgacgcaccgcaatccttacacggggctcacctatgctgaagaccctgtcgtcgctttcgtggaaatcaacaacgagaacggtctcattcacgcttggcttagtggctctattgaccggatgccaaaagtttttcaggatgagttgcaacggcagtggcatgcttggctgaaagcccgatacggcacaacggagaagttgcgcaaagcgtggggtgtcaaggaagagcccttgggcaatgaaatgctgaggaacacgaactttgaggcagggttgcaaaattgggttttggagcgacacgcgggtgctgaagcaacagcggaagttgtcgctgaacccatccctgaacttaaaggtcttcgcttcgtccgcattaatgtcacaaaactgggacaggcaggctggcatgtccaattccatcaaccgaacttgaaagttcaacctgatcgcccatacacactttcgttctgggcgcgagcggaacgaccttgcaccatttcagtcggaatttcgcaagcccacgaaccttggcagaaccttggcttcagtgccgaagtcaaattgacacaggaatggcgtgaatatcggttcaccttcattctcaacaggggtgacgacaacgctcgcgttatcttcagcaacttgggcgctcaaacgacaacttactggttcgccgccccctcccttcgccctggcggaattgttggcttggcagtcaacgagcgccttgaagatggcactgtcccgattttccttcgcggtcgtttcggtgagcgcacacccgaagcacaacgggactggatgcgatttttgtgggagacggaagatcgctattggcaaacaatctaccgctacctcaaggacgagttgaaagtcaaggcattggtcatcggcacaatcgtcggatgcagcacacccaacatgatggcaaaacttgactgcgtggacacacacgcttattggcaacatccgatgtttccctcccgaccatgggaccctgaggattggattgtgcctaaccgaacgatggtcaacgaacgaggcgggactttgcctggcttagcattgcgacgggttttgggcaaaccccattcttgcaccgaatacaaccaccccgcaccaaacacatacagcagcgaagccttccttttgctggcagcatatgcagccttgcaggattgggatgccatctatgccttcagttactcgcaccgacgagatggttgggacttaaggcgcattcccaacttctttgacattgaccaacatcccacgaagatggttacccttatccctgcagcagcgatgtttgttcggggcgatgtgaaacctgccaagcagcaagtggttgtcaagttgacgaaagagcaagaagttgacttgttgcggcgcagttgggcttggttactcgtccacgcaggtcatgtcggagttcctaacgaagcagcactcgtccatcgggttgcaattgcgacagatgggaagcgagttccgccaactgctctcaagcccgaacaagtgaaaattgacggcaaccgatttgtgtccgatactggcgaattgatttgggacttgacggaaaaagggcgcggggttgttatcgttaatgcccagaacagcaaagccgttatcgggttcgcaggtggcaagcggtttgagttgagcagcgtcgtgattgaacctgggcaaactatgcaagacggttggtgcgccatcacactcaccgtcatggacggctccttgccaactcgcccctcgtcccgcgtccctcgtcccgtccgtttgctcatcacggcaacaggctatgcggagaacaccgacatgggttggaaggaagtgcctggttacccacccaaatcaagttgcggtcgcaactggggcaaaccaccttcgttggtggaaggcatctttgccagcatcaccttgccgttacccgccaagcgagtgcaagcgtgggctttggacgaacggggtcaacggaaatcccaaatcccagtcactgccgacccatcaggcaacgccgtcatccgcatcagtccccaatggcaaacgctttggtatgaagttgaggcgcggtaa

SEQ ID NO: 24 - protein, 903 aa, Fervidibacteria bacterium
MRWWMLVILAFISGFGGWWTMRQGLSQEGKQDALLFPFVLPWDDASPSVTNISHWLHKPAGKFGHIRIGADGRLYAGKQRIRFLGVNLCFGACFPRKEDSEKIAARMAKFGINIVRFHHMDMQEFPNGIRRRGVPHTRDLDPEALDRLDYLIAQLKRNGIYVNLNLLVSRPFNAADGLPKEIEQLGWKERHIVGFFYEPCLELQKEYARKLLTHRNPYTGLTYAEDPVVAFVEINNENGLIHAWLSGSIDRMPKVFQDELQRQWHAWLKARYGTTEKLRKAWGVKEEPLGNEMLRNTNFEAGLQNWVLERHAGAEATAEVVAEPIPELKGLRFVRINVTKLGQAGWHVQFHQPNLKVQPDRPYTLSFWARAERPCTISVGISQAHEPWQNLGFSAEVKLTQEWREYRFTFILNRGDDNARVIFSNLGAQTTTYWFAAPSLRPGGIVGLAVNERLEDGTVPIFLRGRFGERTPEAQRDWMRFLWETEDRYWQTIYRYLKDELKVKALVIGTIVGCSTPNMMAKLDCVDTHAYWQHPMFPSRPWDPEDWIVPNRTMVNERGGTLPGLALRRVLGKPHSCTEYNHPAPNTYSSEAFLLLAAYAALQDWDAIYAFSYSHRRDGWDLRRIPNFFDIDQHPTKMVTLIPAAAMFVRGDVKPAKQQVVVKLTKEQEVDLLRRSWAWLLVHAGHVGVPNEAALVHRVAIATDGKRVPPTALKPEQVKIDGNRFVSDTGELIWDLTEKGRGVVIVNAQNSKAVIGFAGGKRFELSSVVIEPGQTMQDGWCAITLTVMDGSLPTRPSSRVPRPVRLLITATGYAENTDMGWKEVPGYPPKSSCGRNWGKPPSLVEGIFASITLPLPAKRVQAWALDERGQRKSQIPVTADPSGNAVIRISPQWQTLWYEVEAR

SEQ ID NO: 25 - DNA, 2685 bp, Teredinibacter turnerae
atgtttttgtttatctcaacgcactcaccacgcctgtgcctgtaccgggcacttctctttccgttgttgctcttctgtgttgatgcgctggcggaagagcgctttgcgcaatcaccgataaagtctttcacgcaaaccgggttggtgcccttcgtgcttcccttcgacgacaatggaacgggtattaccgcttttaataatggttcgcaccagcgcggggaggggcttgccccactcactatcgattccgatgggcatttctccgttgccgggaataggtttaggctttggggcgtgaacattacgggtgactcagcttttccgtcacacgaagacgccgaaaaaatcgccgggcgcttagcaaaatttggcgtgaacattgttcgttttcatcacttggacaataactggggcggtgcgggcctaatcgattatcggcggggagactcccgacatttgagcaaggacaatctcgataagctcgattactttattgccgctctaaaattgcgtggaatctacagcaatataaacctgcttaccgcgcgggaatttttacctgctgatggattacccgcatcgattactcagattgactggaaggcgcggcagatgctcggcgcgatttcgcatgcggttcgcaatctggaaaaagcctacgcgaaaaaacttctgcaccatgtgaacccctacacgcgcctggcataccggacagacccggccatcgcgtttgttgaaataaacaatgaaaacagtctgtttcaacagttttttgacggcaatatagatcgctggccaaaggagtttaaacggccactggcgcaagagtggaatgcttggttgactcgcaagtataaagatcagaatgcactcgagcgcgcttggcaggtgattgataaacctctgggtaacaatctgttaaaaaatgcgaattttgtggccgggttacaggggtggcatttagaccagatcgacggcgcaaaggcgcaggcgagtccgctcgcatccgctggtttgcgtattcaagtagataccgtagggcctgcgttgtggaacattcaactatcccaaaatttacctgaactaaaagacggtgagatttacacactgtcgtttgccgcacgatcggcgtcgcacagtaaaattacaccgctggtgatgcagcgtgcggaaccctggctggtggttgaatcgtttcctgtaaagctcgattcgaaatggcaggaattcagatttcggtttgtgcacagtggatcggcgcaaccattgcgtctgacgttaggtgaattgggttcggtaataggagcgatagacattagggatctgcgcttgcagcccggcgggaccgtcggtgaacttgccgcgaaccaaacgcttgaacgccattctattgggttaaaccgcaacgatgaatcctatctcgcgcagcggcgggaagattggtttgcatttttgtacagccttgagttgacgtactggcaggatatgcaccgctatctggctgaggaactcaaggttaaaagcaatatctatgggactatcgcgagcctgagtccgccttcaattcagcgcgaatttggatttatcgatagccatatctattgggcgcacccacatttccccgctggggcctgggacgcgcagcagtggagtgttgatatgtcgtccatggttaatgcttttccaaacaacacgttgagcgcgctggcgcgccagcgagttgccggcctgccttttgttgtctctgaataccagcatgctatgccaaatccctattctgcagaagggccgcttctggtagcggcctatgcgggtttgcaggattgggacggcgtttatctgttttcttacgaccagggggaactgggttggcaacaggaatttattgacggattctttaaaaccaatttgaatcccgcggcaatggttaattttgccgtcggcggtaatctgtttcgacgtggtgatgtgcagcctgcgcagggcaaacgctggttgaatttttcgcgatcccgcgagctcgcacaaattgcaagcgccggtgcatcctggagcgtgagtccggcggacttcccggcggactggcgtggctacgcatttcatgagcaaataggcttgcagttggaggcgccagcagcggagcctaaactgcctgtgcttgatgttaacaaggtaactgcagatacaggagcgctcacctgggatacgtctgtccaagcgcagggcagggtgacaataaacacggcgaaatctgctggcgtagtgggctttattgcagatcaaggcttccagctcggtgcgctcgaattgagagtgggcgatttgcagatgggttgggccagttggatgatcactgcacaagagggaagtttgcaggatcttgccccaggcgcatcgctgttggcggttgcgacggcaaaaattgagaacagtaaaatgcgttggaacgacgcgcataattcgcttggccgcaattggggtgaagcgccgacgagggttgaggttgtgccctttagtctcacgctgccagttgcttccaggcgggtaaatgcctggtgtctggatgagcgtgggcagcgattgcaggcgttgaaggtcgagcaaacagccacgggcagtcgtatcgacgtggatagcaaagcgcgaacattgtggtatgagatagccatcgcccccaaaatctaa

SEQ ID NO: 26 - protein, 894 aa, Teredinibacter turnerae
MFLFISTHSPRLCLYRALLFPLLLFCVDALAEERFAQSPIKSFTQTGLVPFVLPFDDNGTGITAFNNGSHQRGEGLAPLTIDSDGHFSVAGNRFRLWGVNITGDSAFPSHEDAEKIAGRLAKFGVNIVRFHHLDNNWGGAGLIDYRRGDSRHLSKDNLDKLDYFIAALKLRGIYSNINLLTAREFLPADGLPASITQIDWKARQMLGAISHAVRNLEKAYAKKLLHHVNPYTRLAYRTDPAIAFVEINNENSLFQQFFDGNIDRWPKEFKRPLAQEWNAWLTRKYKDQNALERAWQVIDKPLGNNLLKNANFVAGLQGWHLDQIDGAKAQASPLASAGLRIQVDTVGPALWNIQLSQNLPELKDGEIYTLSFAARSASHSKITPLVMQRAEPWLVVESFPVKLDSKWQEFRFRFVHSGSAQPLRLTLGELGSVIGAIDIRDLRLQPGGTVGELAANQTLERHSIGLNRNDESYLAQRREDWFAFLYSLELTYWQDMHRYLAEELKVKSNIYGTIASLSPPSIQREFGFIDSHIYWAHPHFPAGAWDAQQWSVDMSSMVNAFPNNTLSALARQRVAGLPFVVSEYQHAMPNPYSAEGPLLVAAYAGLQDWDGVYLFSYDQGELGWQQEFIDGFFKTNLNPAAMVNFAVGGNLFRRGDVQPAQGKRWLNFSRSRELAQIASAGASWSVSPADFPADWRGYAFHEQIGLQLEAPAAEPKLPVLDVNKVTADTGALTWDTSVQAQGRVTINTAKSAGVVGFIADQGFQLGALELRVGDLQMGWASWMITAQEGSLQDLAPGASLLAVATAKIENSKMRWNDAHNSLGRNWGEAPTRVEVVPFSLTLPVASRRVNAWCLDERGQRLQALKVEQTATGSRIDVDSKARTLWYEIAIAPKI

SEQ ID NO: 27 - DNA, 2685 bp, Teredinibacter turnerae
atgttttggtttacctctgcgtactcgcgaagcttgtgtctgtaccgggcacttctgtttccggtgttgctcttctgtgttgatgctccggcagcagagcgtctttcgcaaccaccaacaaaatcggtaaatcacacagggttagcaccgttcgtgcttcccttcgacgatgatggtacgggtattaccgcttttaataacggatcgcaccagcgaggggaggggcttgctccgctcactatcgattccgccggacatttctccgtcgccgggaataggtttaggctttggggcgtaaatataacgggggactccgccttcccctcacacaaggatgcggaaaaagttgccggacgtttagcaaaattcggcgtgaacattgttcggtttcatcatttggacaataactggggtggtgcgggcctaattgattatcgacggggagactcccgtcatttgagtaaggaaaatctcgataagctcgattattttattgccgctttaaaattgcgtggaatctacagcaatataaacctgcttactgcgcgcgaatttttacctgctgacggattgcctgcatcgattactcaaatcgattggaaggcgcggcaaatgctcggcgcgatttctccctcggtccgcaatttggaaaaagcctacgcgaaacaaattctgaagcatgtaaatccgtacacgcgcctggcgtaccgggtagatccggccatcgcatttgttgaaataaacaatgaaaacagcctgtttcaacagttttttgacggcaatatagaccgctggccggaagcgtttagtcagccactggcgcaagagtggaatgcttggttggctcgcaagtataaagatcacgctgcactcgagcgtgcctggcaggtgattgataaacctctgggcaacaatctgttaaaaaatgcggattttgtggcgggcttacagggttggcatttagaccagatcgacggcgcaaaggcgcaggccaatccgctcgcatccgctggtttgcgtatccaaatagataccgtagggcctgcgttatggaacattcaactttcccaaaacttacctgaactcaaagacggtgagatttacacattgtcgtttgccgcacgctcgcagtcgcacagcagaattacacctctgctgatgcagagtgtggaaccctggcaggtggtcgaatccttccctgtgaagctcgattctgaatggcgggaattcaggtttcagtatgtgcatactggatcagcgcaaccgttgcgcttaacgttgggtgaattgggttcggtaataggggcaatagatgttagggatcttcgcctgcagtctggcggaactgtcggtgaacttgctaagaaccagactctcgaacgccgttctataggattaaaccgtaacgatgaatcctatctcgcgcagcggcgggaagactggtttgcatttttgtacagccttgagctggcgtactggcaggatatgcacagctatttggctgatgaactcaaggttaaaaacaatatttatgggactatcgcgagcttgagtccgccttcaattcagcgcgaatttggatttatcgatagtcatatctattgggcgcacccacattttcccgctggagcctgggatgcacagcagtggagtgtcgatatgtcgtccatggttaacgccttcccaaacaacacgttaagcgctctggcgcgccagcgcgtggccgggctgccttttgtagtctctgaatatcagcatgctatgccaaatccctattctgcagaaggcccgctgctggtagcggcctatgcgggtttgcaggattgggatggcgtttatctgttttcgtacgatcagggggaactgggttggcaacaggaatttatcgacggattctttaaaactaatttgaacccagcggcaatggttaattttgccgtcggcgggaatctgtttcgacgtggagatgtgcagcctgcgcagggcaaacgctggttaaatttttctcgatccggcgagctcgcacaaattacaaacgccggtgcatcttggagtgtgagtccggcggacttccctccggaatggcgtggctacgcttttcacgagcaaatgggcttgcagttgggtatgtcaacgacggagtctaaaccgccggtgcttgatgttaacaaagtgactgccgagacgggcgaactcagttgggatacaactagccaagcgcagggtagggtgacaataaacacagaaaaatccgctggcgtagtgggctttgttgcagatcaacacttccagctcggtgcgttggggttgaccttgggcgatctacagatgggctgggccagctggatggtcactgcgcaagagggtagtttgcaggatcttgcagcaggcgcttcgctgttggcagttgcgaccgcaaaaattgaaaacagtaaaatgcgctggaacgacgctcataattcgctcggccgcaattggggtgaagcgccgacgagggttgaagttgtgccttttagtctctcgttgccaattgctgccaggcgggttagtgcctggtgtttggatgagcgtggtcagcgaatgcggtcgttgaaggttgtgcaaacagcgacaggcagtcgtatcgatgtggatagcaaagcgcgaacattatggtacgagatcgtcataaccccgaaaatataa

SEQ ID NO: 28 - protein, 894 aa, Teredinibacter turnerae
MFWFTSAYSRSLCLYRALLFPVLLFCVDAPAAERLSQPPTKSVNHTGLAPFVLPFDDDGTGITAFNNGSHQRGEGLAPLTIDSAGHFSVAGNRFRLWGVNITGDSAFPSHKDAEKVAGRLAKFGVNIVRFHHLDNNWGGAGLIDYRRGDSRHLSKENLDKLDYFIAALKLRGIYSNINLLTAREFLPADGLPASITQIDWKARQMLGAISPSVRNLEKAYAKQILKHVNPYTRLAYRVDPAIAFVEINNENSLFQQFFDGNIDRWPEAFSQPLAQEWNAWLARKYKDHAALERAWQVIDKPLGNNLLKNADFVAGLQGWHLDQIDGAKAQANPLASAGLRIQIDTVGPALWNIQLSQNLPELKDGEIYTLSFAARSQSHSRITPLLMQSVEPWQVVESFPVKLDSEWREFRFQYVHTGSAQPLRLTLGELGSVIGAIDVRDLRLQSGGTVGELAKNQTLERRSIGLNRNDESYLAQRREDWFAFLYSLELAYWQDMHSYLADELKVKNNIYGTIASLSPPSIQREFGFIDSHIYWAHPHFPAGAWDAQQWSVDMSSMVNAFPNNTLSALARQRVAGLPFVVSEYQHAMPNPYSAEGPLLVAAYAGLQDWDGVYLFSYDQGELGWQQEFIDGFFKTNLNPAAMVNFAVGGNLFRRGDVQPAQGKRWLNFSRSGELAQITNAGASWSVSPADFPPEWRGYAFHEQMGLQLGMSTTESKPPVLDVNKVTAETGELSWDTTSQAQGRVTINTEKSAGVVGFVADQHFQLGALGLTLGDLQMGWASWMVTAQEGSLQDLAAGASLLAVATAKIENSKMRWNDAHNSLGRNWGEAPTRVEVVPFSLSLPIAARRVSAWCLDERGQRMRSLKVVQTATGSRIDVDSKARTLWYEIVITPKI

SEQ ID NO: 29 - DNA, 2868 bp, Diplosphaera colitermitum
atgtctccccagattgcctctttcgcacgtcgctgccgcctcgtccacacactatctggtgcactgcttgccatcgcatttgcgtccggcccgctgacggcgatcatcgccaacgctggcgcatctgaactcacggcggccacggccaaccaactccccttcgccaacggcttcccctttcacatcccccccaccggcacccaaccgggcaccgcgccctacgacctcgcgatcccgccgtccgccaacacaaaaatcgacagccccatcagcattcgcggcgaccaattcatcgttcgccatttgacatcaaacaccagccccgacaccggcgaacccatccgcttctggggcaccaacctttgcttttccggcgtattccccgaacacgacatcgctgaccgcatggccgcgcgcatggccacactcggcatcaacatcgtccgccttcaccacttcgaccaacgccgtttccccggcggcatctggcatcgcgacgcccccggcgcctccaaatctcccaacgaagacgacatcgcccaccaaaccttcgacccagaatcccttgaccgcctcgactacctcatcgccgccctcaaaaaacgcggcatctacaccaacctaaacctcaaagtctcccgcatattcagtcccgaccacgacggccccgacttcccgaaacccgatcctgccaaaaacgaaatcctgcccaaaaaaggcaaaggctttgaccaattttacacccccgccatcgccgcgcaaaaagactacgcccgccgcctcctcacccatcgcaacccctacaccggcctcacctacaccgaagaccccgccgtcgcgatggtggagatcaacaacgaaaacggcatcctctgggcctggaactaccgaatccttgaccgcattccctcccgtttcattgacgaactcgccgcccgctggaacacctggcttcgtaaccaatattccactaccgacgcactccgcgccgcatggaatccggcgagtggcgcgggcgtcccacccgcaaccggaaccgtagcctcccggtccgttccctccaccggtggcaacctcttggaaaacatcccccccgccctcttcaccgccaaaaaagcccgcgccaccctcgccccgctcaccgccgccgccgacgccgacgattccaccccggcctcccgtcgcctcaccgttgccgaagtccctgctgccaccgcttggaatgtccgctgcaactggcccctcccgaccgcgcttcccgccgacgccacctacaccgccacccttcgcctccgcgcaaaccaaccccacaaaatcaaactccgcctccgctccccgtccgacaacaaggacctcgcgcccgtccgcaccctcaacctcgccaccgagtggaaaaaccacagcaccacctttgccatcccgccgggtgacgccgccgtcgccgcccaactcaccctcgaagcgggcatccccggcctcgtcctcgacatcgactccgcttcccttcaaccgctcaccagcaaaaacctcctcggcctcccagcgggccaaggtctcgtctcccaaagtggcgcgggcgggacgcccgcgccacgccccgtcgaatgggtattccgccgcgatctcccctcccgcacacccgccgtcgtcaccgacgtcatgcgcttcctccgcgacaccgaaatcgcctactggcgcgaaatgcacgctttcttacgcaacgacctccgcgtcgccgcacccataaccaccaccgccgttggctacaccacgccgcaaatcgccgccgaaaccgccgacttcatcgacacccaccgctactggggatcaccccgctttcccgcctttgaccggacaaaaccctggaccgtgcaacaaaaacccatggtctcccaccccgcccagtccaccatcgaacgcatgtccgcccgacgtgtattcggaaaaccattcaccatcaccgaatacaaccacccgccctccaccgaccaccacgccgaggccttccccctcgtcggcgtctggggcgccgcgcaaggctgggacggcctcttccagttcgcctactcccacagccgcgcttgggaagctgacatcatgaccggcttcttcgacaccgagcccaaccccgcgcacaccgtcgccgccctcgccgcctccgacatcttccgtcaccgccgcatcaccccctttgcctccacaaaaaccggctacgtcaccctagaccgccaactcgaacgccagaacaactacgccttccctcgcgaaatcgaggccgacgccatctacggaggactcccgcccgacgcctggcttaccaaccgcgtcggcctcgcgccaagtgacgcgggcgtcccgcccgcaacccttgcccctccgccctccgtctcccaaagcctcgtctgggacgccgccaaccccgccaccgcccacgtccgttacacgggcgacggcgttgctggcctcatcggattcgtttccggccagaccctcgatctcggatggctccgtatcacacccggcactacatcactcaacggcttctcaattgtcatgctcaataccgttgaccgccaagccctcggagcccccggacgctacctgctcactgtggccgtccgagcctccaacctcggcatgggctggaacgctgaccgcaccggcttcggcaaaaaatggggcactggtcccacccatgccgaaaccgccccgatcgcgctcgatttcgcctctgccaccggcgtccgggtgtatccgctcaatcccgacggaacacgtcgcccggaactcccgccggtctccctcccgggtcgcttcgaagccactcctgccagcaaaactctctggtacgaaatcatcctcccgtaa

SEQ ID NO: 30 - protein, 955 aa, Diplosphaera colitermitum
MSPQIASFARRCRLVHTLSGALLAIAFASGPLTAIIANAGASELTAATANQLPFANGFPFHIPPTGTQPGTAPYDLAIPPSANTKIDSPISIRGDQFIVRHLTSNTSPDTGEPIRFWGTNLCFSGVFPEHDIADRMAARMATLGINIVRLHHFDQRRFPGGIWHRDAPGASKSPNEDDIAHQTFDPESLDRLDYLIAALKKRGIYTNLNLKVSRIFSPDHDGPDFPKPDPAKNEILPKKGKGFDQFYTPAIAAQKDYARRLLTHRNPYTGLTYTEDPAVAMVEINNENGILWAWNYRILDRIPSRFIDELAARWNTWLRNQYSTTDALRAAWNPASGAGVPPATGTVASRSVPSTGGNLLENIPPALFTAKKARATLAPLTAAADADDSTPASRRLTVAEVPAATAWNVRCNWPLPTALPADATYTATLRLRANQPHKIKLRLRSPSDNKDLAPVRTLNLATEWKNHSTTFAIPPGDAAVAAQLTLEAGIPGLVLDIDSASLQPLTSKNLLGLPAGQGLVSQSGAGGTPAPRPVEWVFRRDLPSRTPAVVTDVMRFLRDTEIAYWREMHAFLRNDLRVAAPITTTAVGYTTPQIAAETADFIDTHRYWGSPRFPAFDRTKPWTVQQKPMVSHPAQSTIERMSARRVFGKPFTITEYNHPPSTDHHAEAFPLVGVWGAAQGWDGLFQFAYSHSRAWEADIMTGFFDTEPNPAHTVAALAASDIFRHRRITPFASTKTGYVTLDRQLERQNNYAFPREIEADAIYGGLPPDAWLTNRVGLAPSDAGVPPATLAPPPSVSQSLVWDAANPATAHVRYTGDGVAGLIGFVSGQTLDLGWLRITPGTTSLNGFSIVMLNTVDRQALGAPGRYLLTVAVRASNLGMGWNADRTGFGKKWGTGPTHAETAPIALDFASATGVRVYPLNPDGTRRPELPPVSLPGRFEATPASKTLWYEIILP

SEQ ID NO: 31 - DNA, 2685 bp, Teredinibacter turnerae
atgttttggtttacctatgcgtactcgcgaagcttgtgtctgtaccgggcactcctgtttccgttgttgctcttctgtgttgatgctccggcagcagagcgtctttcgcaatcaccgacaaaatcggtaaatcacacagggttagcaccgttcgtgcttcccttcgacgatgatggtacgggtattaccgcttttaataacgggtcgcaccagcgaggggagaggcttgctccgctcactatcgattccgccggacatttctccgtcgccgggaataggtttaggctttggggcgtaaatataacaggggattccgccttcccctcacacaaggatgcggaaaaagttgccggacgtttagcaaaatttggcgtgaacattgttcggtttcatcatttggacaataactggggtggtgcgggcctaattgattatcgacggggagactcccgtcatttgagtaaggaaaatctcgataagctcgattattttattgccgctttaaaatcgcgtggaatctacagcaatataaacctgcttaccgcgcgcgaatttttacctgctgacggattgcctgcatcgattactcaaatcgattggaaggcgcggcaaatgctcggcgcgatttctcccgcggtccgcaatttggaaaaagcctacgcgaaacaaattctgcaacatgtaaatccgtacacgcgcttggcgtatcgggtagatccggccatcgcttttgttgaaataaacaatgaaaacagcctgtttcaacagttttttgacggcaatatagaccgctggccggaagcgtttagtcagccactggcgcaagagtggaatgcttggttggctcgcaagtataaagatcacgctgcactcgagcgtgcctggcaggtgattgataaacctctgggcaacaatctgttaaaaaatgcggattttgtggcgggcttacagggttggcatttagaccagatcgacggcgcaaaggcgcaggccaatccgctcgcatccgctggtttgcgtatccaaataaataccgtagggcctgcgttatggaacattcaactttctcaaaacttacctgaactcaaagacggtgagatttacacattgtcgtttgccgcacgctcgcagtcgcacagcagaattacacctctgctgatgcagagtgtggcaccctggcaggtggtcgaatctttccctgtgaagctcgattccgaatggcaggaattcaggtttcagtatgtgcatactggatcagcgcaaccgttgcgcctaacgttgggtgaattgggttcggtaataggggcaatagatgttagggatcttcgcctgcagtctggcggaactgttggtgaacttgctaagaaccagactctcgaacgccgttctataggattaaaccgtaacgatgaatcctatctcgcgcagcggcgggaagactggtttgcatttttgtacagccttgagctggcgtactggcaggatatgcacagctatttggctgatgaactcaaggttaaaaacaatatttatgggactatcgcgagcttgagtccgccttcaatacagcgcgaatttggatttatcgatagtcatatctattgggcgcacccacattttcccgctggagcctgggatgcacagcagtggagtgtcgatatgtcgtccatggttaacgccttcccaaacaacacgttaagcgctctggcgcgccagcgcgtggccgggctgccttttgtagtctctgaatatcagcatgctatgccaaatccctattctgcagaaggcccgctgctggtagcggcctatgcgggtttgcaggattgggacggcgtttatctgttttcgtacgatcagggggaactgggttggcaacaggaatttatcgatggattctttaaaaccaatttgaacccagcggcaatggttaattttgccgtcggcggtaatctgtttcgacgtggagatgtgcagcctgcgcatggcaaacgctggttaaatttttcgcgatccggcgagctcgcacgaattacaaacgccggtgcatcttggagtgtgagtccggcggacttccctccggaatggcgtggctacgcttttcacgagcaaatgggcttgcagttggatatgtcagggagggagtctaaaccgccggtgcttgatgttaacaaagtgactgccgagacgggcgaactcagttgggatacaactatccaagcgcagggtagggtgacaataaacacagcgaaatccgctggcgtagtgggctttgttgcagatcaacacttccagctcggtgcgttggggttgaccttgggcgatctacagatgggttgggccagttggatggtcactgcgcaagagggtagtttgcaggatcttgcagcaggcgcttcgctgttggcagttgcgaccgcaaaaattgaaaacagtaaaatgcgttggaacgacgctcataattcgctcggccgcaattggggggaagcgccgacgagagttgaagttgtgccttttagtctctcgttgccaattgctgccaggcgggttagtgcctggtgtttggatgagcgtggtcagcgaatgcagtcgctgagggttgtgcaaacagcgacaggcagtcgtatcgatgtggatagcaaagcgcgaacattatggtacgagatcgtcataaccccgaaaatataa

SEQ ID NO: 32 - protein, 894 aa, Teredinibacter turnerae
MFWFTYAYSRSLCLYRALLFPLLLFCVDAPAAERLSQSPTKSVNHTGLAPFVLPFDDDGTGITAFNNGSHQRGERLAPLTIDSAGHFSVAGNRFRLWGVNITGDSAFPSHKDAEKVAGRLAKFGVNIVRFHHLDNNWGGAGLIDYRRGDSRHLSKENLDKLDYFIAALKSRGIYSNINLLTAREFLPADGLPASITQIDWKARQMLGAISPAVRNLEKAYAKQILQHVNPYTRLAYRVDPAIAFVEINNENSLFQQFFDGNIDRWPEAFSQPLAQEWNAWLARKYKDHAALERAWQVIDKPLGNNLLKNADFVAGLQGWHLDQIDGAKAQANPLASAGLRIQINTVGPALWNIQLSQNLPELKDGEIYTLSFAARSQSHSRITPLLMQSVAPWQVVESFPVKLDSEWQEFRFQYVHTGSAQPLRLTLGELGSVIGAIDVRDLRLQSGGTVGELAKNQTLERRSIGLNRNDESYLAQRREDWFAFLYSLELAYWQDMHSYLADELKVKNNIYGTIASLSPPSIQREFGFIDSHIYWAHPHFPAGAWDAQQWSVDMSSMVNAFPNNTLSALARQRVAGLPFVVSEYQHAMPNPYSAEGPLLVAAYAGLQDWDGVYLFSYDQGELGWQQEFIDGFFKTNLNPAAMVNFAVGGNLFRRGDVQPAHGKRWLNFSRSGELARITNAGASWSVSPADFPPEWRGYAFHEQMGLQLDMSGRESKPPVLDVNKVTAETGELSWDTTIQAQGRVTINTAKSAGVVGFVADQHFQLGALGLTLGDLQMGWASWMVTAQEGSLQDLAAGASLLAVATAKIENSKMRWNDAHNSLGRNWGEAPTRVEVVPFSLSLPIAARRVSAWCLDERGQRMQSLRVVQTATGSRIDVDSKARTLWYEIVITPKI

SEQ ID NO: 33 - DNA, 2682 bp, Teredinibacter turnerae
atgtttttgtttatctcaacgcactcaccaagcctgtgcctgtaccgggcacttctctttccgttgttgctcttctgtgttgatgcgctggcggaagagcgccttgcacaatcaccgataaagtctttcacgcaaaccgggttggtgcccttcgtgcttcccttcgacgacaatggagcgggtattaccgcttttaataatggttcgcaccagcgcggggaggggcttgccccgctcactatcgattccgacgggcatttctccgttgccgggaataggtttaggctttggggcgtgaacattacgggtgactcagcttttccgtcacacgaagacgccgaaaaaatcgccgggcgtttagcaaaatttggcgtgaacattgttcgttttcatcacttggacaataactggggcggtgcgggcctaatcgattatcggcggggagactcccgacatttgagcaaggacaatctcgataagctcgattactttattgccgctttaaaattgcgtggaatctacagcaatataaacctgcttaccgcgcgggaatttttacctgctgatggattacccgcatcgattactcagattgactggaaggcgcggcagatgctcggcgcgatttcgcctgcggttcgcaatctggaaaaagcctacgcgaaaaaaattctgcatcatgtgaacccctacacgcgcctggcataccgggcagacccggccatcgcgtttgttgaaataaacaatgaaaacagtctgtttcaacagttttttgacggcaatatagatcgctggccaaaggagtttaaacggccactggcgcaagagtggaatgcttggttgactcgaaagtataaagatcagaatgcactcgagcgcgcctggcaggtgattgataaacctctgggtaacaatctgttaaaaaatgcgaattttgtggccgggttacaggggtggcatttagaccagatcgacggcgcaaaggcgcaggcgagtccgctcgcatccgcgggtttgcgtattcaagtagataccgtagggcctgcgttgtggaacattcaactatcccaaaacttacctgaactaaaagacggtgagatttacacactgtcgtttgccgcacgatcggcgtcgcacagtcaaattacaccgctggtgatgcagcgtgcggaaccctggcaggtggttgaatcgtttcctgtaaagctcgattcgaaatggcaggaattcagatttcggtttgtgcacagtggatcggcgcaaccattgcgtctgacgttaggtgaattgggttcggtaataggagcgatagacattagggatctgcgcttgcagcccggcgggaccgtcggtgaacttgccgcgaaccaaacgcttgaacgccattctattgggttaaaccgcaacgatgaatcctatctcgcgcagcggcgggaagattggtttgcatttttgtacagccttgagttgacgtactggcaggatatgcaccgctatctggctgaggaactcaaggttaaaagcaatatctatgggactatcgcgagcctgagtccgccttcaattcagcgcgaatttggatttatcgatagccatatctattgggcgcacccacatttccccgctggggcctgggacgcgcagcagtggagtgttgatatgtcgtccatggttaatgcttttccaaacaacacgttgagcgcgctggcgcgccagcgagttgccggcctgccttttgttgtctctgaataccagcatgctatgccaaatccctattctgcagaagggccgcttctggtagcggcctatgcgggtttgcaggattgggacggcgtttatctgttttcttacgaccagggggaactgggttggcaacaggaatttattgacggattctttaaaaccaatttgaatcccgcggcaatggttaattttgccgtcggcggtaatctgtttcgacgtggtgatgtgcagcctgcgcagggcaaacgctggttaaatttttcgcgatcccgcgagctcgcacaaattgcaagcgccggtgcatcctggagcgtgagtccggcggacttcccggcggactggcgtggctacgcatttcatgagcaaataggcttgcagttggaggcgccagcagcggagcctaaactgcctgtgcttgataacaaggtaactgcagatacaggagcgctcacctgggatacgtctgtccaagcgcagggcagggtgacaataaacacggcgaaatctgctggcgtagtgggctttattgcagatcaaggcttccagctcggtgcgctcgaattgagagtgggcgatttgcagatgggttgggccagttggatgatcactgcacaagagggaagtttgcaggatcttgccccaggcgcatcgctgttggcggttgcgacggcaaaaattgagaacagtaaaatgcgttggaacgacgcgcataattcgcttggccgcaattggggtgaagcgccgacgagggttgaggttgtgccctttagtctcacgctgccagttgcttccaggcgggtaaatgcctggtgtctggatgagcgtgggcagcgattgcacgcgttgaaggtcgagcaaacagccacgggcagtcgtatcgacgtggatagcaaagcgcgaacattgtggtatgagatagccatcgcccccaaaatctaa

SEQ ID NO: 34 - protein, 893 aa, Teredinibacter turnerae
MFLFISTHSPSLCLYRALLFPLLLFCVDALAEERLAQSPIKSFTQTGLVPFVLPFDDNGAGITAFNNGSHQRGEGLAPLTIDSDGHFSVAGNRFRLWGVNITGDSAFPSHEDAEKIAGRLAKFGVNIVRFHHLDNNWGGAGLIDYRRGDSRHLSKDNLDKLDYFIAALKLRGIYSNINLLTAREFLPADGLPASITQIDWKARQMLGAISPAVRNLEKAYAKKILHHVNPYTRLAYRADPAIAFVEINNENSLFQQFFDGNIDRWPKEFKRPLAQEWNAWLTRKYKDQNALERAWQVIDKPLGNNLLKNANFVAGLQGWHLDQIDGAKAQASPLASAGLRIQVDTVGPALWNIQLSQNLPELKDGEIYTLSFAARSASHSQITPLVMQRAEPWQVVESFPVKLDSKWQEFRFRFVHSGSAQPLRLTLGELGSVIGAIDIRDLRLQPGGTVGELAANQTLERHSIGLNRNDESYLAQRREDWFAFLYSLELTYWQDMHRYLAEELKVKSNIYGTIASLSPPSIQREFGFIDSHIYWAHPHFPAGAWDAQQWSVDMSSMVNAFPNNTLSALARQRVAGLPFVVSEYQHAMPNPYSAEGPLLVAAYAGLQDWDGVYLFSYDQGELGWQQEFIDGFFKTNLNPAAMVNFAVGGNLFRRGDVQPAQGKRWLNFSRSRELAQIASAGASWSVSPADFPADWRGYAFHEQIGLQLEAPAAEPKLPVLDNKVTADTGALTWDTSVQAQGRVTINTAKSAGVVGFIADQGFQLGALELRVGDLQMGWASWMITAQEGSLQDLAPGASLLAVATAKIENSKMRWNDAHNSLGRNWGEAPTRVEVVPFSLTLPVASRRVNAWCLDERGQRLHALKVEQTATGSRIDVDSKARTLWYEIAIAPKI

SEQ ID NO: 35 - DNA, 2685 bp, Teredinibacter turnerae
atgtttttgtttatctcaacgcactcaccacgcctgcgcctgtaccgggcacttctctttccgttgttgctcttctgtgttgatgcgctggcggaagagcgccttgcgcaatcaccgataaagtctttcactcaaaccgggttggtgcccttcgtgcttcccttcgacgacaatggagcgggtattaccgcttttaataatggttcgcaccagcgcggggaggggcttgccccactcactatcgattccgatgggcatttctccgttgccgggaataggtttaggctttggggcgtgaacattacgggtgactcagcttttccgtcacacgaagacgccgaaaaaatcgccgggcgtttagcaaaatttggcgtgaacattgttcgttttcatcacttggacaataactggggcggtgcgggcctaatcgattatcggcggggagactcccgacatttgagcaaggacaatctcgataagctcgattactttattgccgctctaaaattgcgtggaatctacagcaatataaacctgcttaccgcgcgggaatttttacctgctgatggattacccgcatcgattactcagattgactggaaggcgcggcagatgctcggcgcgatttcgcctgcgattcgcaatctggaaaaagcctacgcgaaaaaacttctgcaccatgtgaacccctacacgcgcctggcataccggacagacccggccatcgcgtttgttgaaataaacaatgaaaacagtctgtttcaacagttttttgacggcaatatagatcgctggccaaaggagtttaaacggcaactggcgcaagagtggaatgcttggttgactcgcaagtataaagatcacaatgcactcgagcgcgcctggcaggtgattgataaacctctgggtaacaatctgttaaaaaatgcgaactttgtggccgggttacagggttggcatttagaccagatcgacggcgcaaaggcgcaggcgagtccgctcgcatccgcgggtttgcgtattcaagtagataccgtagggcctgcgttgtggaacattcaactatcccaaaacttacctgaactaaaagacggtgagatttacacactgtcgtttgccgcacgatcggcgtcgcacagtcaatttacaccgctggtgatgcagcgtgcggaaccctggcaggtggttgaatcgtttcctgtaaagctcgattcgaaatggcaggaattcagatttcggtttgtgcacagtggatcggcgcaaccattgcgtctgacgttaggtgaattgggttcggtaataggagcgatagacattatggatctgcgcttgcagcccggcgggagcgtcggtgaacttgccgctaaccaaacgcttgaacgccattctattgggttaaaccgcaacgatgaatcctatctcgcgcagcggcggaaagattggtttgcatttttgtacagccttgagttgacgtactggcaggatatgcaccgttatctggctgaggaactcaaggttaaaagcaatatttatgggactatcgcgagcttgagtccgccttcaattcagcgcgaatttggatttatcgatagtcatatctattgggcgcacccacatttccccgctggggcctgggacgcgcagcagtggagtgttgatatgtcgtccatggttaatgcttttccaaacaatacgttgagcgcgctggcgcgccaacgagttgccggcctgccttttgttgtctctgaataccagcatgctatgccaaatccctattctgcagaagggccgcttctggtagcggcctatgcgggtttacaggattgggacggcgtttatctgttttcttacgaccagggtgaactgggttggcaacaggaatttattgacggattctttaaaaccaatttgaatcccgcggcaatggttaattttgccgtcggcggtaatctgtttcgacgtggtgatgtgcagcctgcgcagggcaaacgctggttaaatttttcgcgatcccgcgagctcgcacaaattgcaagcgccggtgcatcctggagcgtgagtccggcggacttcccggcggactggcgtggctacgcatttcatgagcaaataggcttgcagttggaggcgccagcagcggagcctaaactgcctgtgcttgatgttaacaaggtaactgcagatacaggagcgctcacctgggatacgtctgtccaagcgcagggcagggtgaaaataaacacggcgaaatccgctggcgtagtgggctttattgcagatcaaggcttccagctcggtgcgctcgaattgagagtgggcgatttgcagatgggttgggccagttggatgatcactgcacaagagggaagtttgcaggatctcgcccaaggcgcatcgctgttggcggttgcgacggcaaaaattgagaacagtaaaatgcgctggaacggcgcgcataattcgcttggccgcaattggggtgaagcgccgacgagggttgaggttgtgccttttagtctcacgctgccaattgcttccaggcgggtaaatgcttggtctctggatgagcgtgggcagcgattgcacgcgttgaaggtcgagcaaatagccacgggcagtcgtatcgacgtggatagcaaagcgcgaacattgtggtatgagatagccatcgcccccaaaatctaa

SEQ ID NO: 36 - protein, 894 aa, Teredinibacter turnerae
MFLFISTHSPRLRLYRALLFPLLLFCVDALAEERLAQSPIKSFTQTGLVPFVLPFDDNGAGITAFNNGSHQRGEGLAPLTIDSDGHFSVAGNRFRLWGVNITGDSAFPSHEDAEKIAGRLAKFGVNIVRFHHLDNNWGGAGLIDYRRGDSRHLSKDNLDKLDYFIAALKLRGIYSNINLLTAREFLPADGLPASITQIDWKARQMLGAISPAIRNLEKAYAKKLLHHVNPYTRLAYRTDPAIAFVEINNENSLFQQFFDGNIDRWPKEFKRQLAQEWNAWLTRKYKDHNALERAWQVIDKPLGNNLLKNANFVAGLQGWHLDQIDGAKAQASPLASAGLRIQVDTVGPALWNIQLSQNLPELKDGEIYTLSFAARSASHSQFTPLVMQRAEPWQVVESFPVKLDSKWQEFRFRFVHSGSAQPLRLTLGELGSVIGAIDIMDLRLQPGGSVGELAANQTLERHSIGLNRNDESYLAQRRKDWFAFLYSLELTYWQDMHRYLAEELKVKSNIYGTIASLSPPSIQREFGFIDSHIYWAHPHFPAGAWDAQQWSVDMSSMVNAFPNNTLSALARQRVAGLPFVVSEYQHAMPNPYSAEGPLLVAAYAGLQDWDGVYLFSYDQGELGWQQEFIDGFFKTNLNPAAMVNFAVGGNLFRRGDVQPAQGKRWLNFSRSRELAQIASAGASWSVSPADFPADWRGYAFHEQIGLQLEAPAAEPKLPVLDVNKVTADTGALTWDTSVQAQGRVKINTAKSAGVVGFIADQGFQLGALELRVGDLQMGWASWMITAQEGSLQDLAQGASLLAVATAKIENSKMRWNGAHNSLGRNWGEAPTRVEVVPFSLTLPIASRRVNAWSLDERGQRLHALKVEQIATGSRIDVDSKARTLWYEIAIAPKI

SEQ ID NO: 37 - DNA, 2685 bp, Teredinibacter turnerae
atgtttttgtttatctcaacgcactcaccacgcctgcgcctgtaccgggcacttctctttctgttgttgctcttctgtgttgatgcgctggcggaagagcgccttgcggaatcaccgataaagtctttcactcaaaccgggttggtgcccttcgtgcttcccttcgacgacaatggagcgggtattaccgcttttaataatggttcgcaccagcgcggggaggggcttgccccactcactatcgattccgacgggcatttctccgtcgccgggaataggtttaggctttggggcgtgaacattacgggtgactcagcttttccatcacacgaagacgccgaaaaaattgccgggcgtttagcaaaatttggcgtgaacattgttcgttttcatcacttggacaataactggggcggtgcgggcctaatcgattatcggcggggagactcccgacatttgagcaaggataatctcgataagctcgattactttattgccgctttaaaattgcgtggaatctacagcaatataaacctgcttaccgcgcgggaatttttacctgctgatgaattacccgcatcgattactcagattgactggaaggcgcggcagatgcttggcgcgatttcgcctacggttcgcaatctggaaaaagcctacgcgaaaaaacttctgcaccatgtgaacccctacacgcgccttgcataccgggcagacccggccatcgcgtttgttgaaataaacaatgaaaacagtctgtttcaacagttttttgacggcaatatagatcgctggccaaaggagtttaaacggccactggcgcaagagtggaatgcttggttgactcgcaagtataaagatcagaatgcactcgagcgcgcctggcaggtgattgataaacctctgggtaacaatctgttaacaaatgcgaattttgtggccgggttacaggggtggcacttagaccagatcgacggcgcaaaggcgcaggcgagtccgctcgcatccgcgggtttgcgtattcaagtagataccgtagggcctgcgttgtggaacattcaactatcccaaaacttacctgaactaaaagacggtgagatttacacactgtcgtttgccgcacgatcggcgtcgcacagtcaaattacaccgctggtgatgcagcgtgcggaaccctggcaggtggttgaatcgtttcctgtaaagctcgattcgaaatggcaggaattcagatttcggtttgtgcacagtggatcggcgcaaccattgcgtctgacgttaggtgaattgggttcggtaataggagcgatagacattagggatctgcgtttgcagcccggcgggactgtcggtgaacttgccgcgaaccaaacgcttgaacgccattctattgggttaaaccgcaacgatgaatcctatctcgcgcagcggcgggaagattggtttgcatttttgtacagccttgagttgacgtactggcaggatatgcaccgctatctggctgaggaactcaaggttaaaagcaatatctatgggactatcgcgagcctgagtccgccttcaattcagcgcgaatttggatttatcgatagccatatctattgggcgcacccacatttccccgctggggcctgggacgcgcagcagtggagtgttgatatgtcgtccatggttaatgcttttccaaacaacacgttgagcgcgctggcgcgccagcgagttgccggcctgccttttgttgtctctgaataccagcatgctatgccaaatccctattctgcagaagggccgcttctggtagcggcctatgcgggtttgcaggattgggacggtgtttatctgttttcttacgaccagggggaactgggttggcaacaggaatttattgacggattctttaaaaccaatttgaatcccgcggcaatggttaattttgccgtcggcggtaatctgtttcgacgtggtgatgtgcagcctgcgcagggcaaacgctggttaaatttttcgccatcccgcgagctcgcacaaattgcaagcgccggtgcatcctggagcgtgagtccggcgaacttcccggcggactggcgtggctacgcatttcatgagcaaataggcttgcagttggaggcgccagcagcggagcctaaactgcctgtgcttgatgttaacaaggtaactgcagatacaggagcgctcacctgggatacgtctgtccaagcgcagggcagggtgacaataaacacggcgaaatctgctggcgtagtgggctttattgcagatcaaggcttccagctcggtgcgctcgaattgagagtgggcgatttgcagatgggttgggccagttggatgatcactgcacaagagggaagtttgcaggatcttgccccaggcgcatcgctgttggcggttgcgacggcaaaaattgagaacagtaaaatgcgttggaacgacgcgcataattcgcttggccgcaattggggtgaagcgccgacgagggttgaggttgtgccctttagtctcacgctgccagttgcttccaggcgggtaaatgcctggtgtctggatgagcgtgggcagcgattgcacgcgttgaaggtcgagcaaacagccacgggcagtcgtatcgacgtggatagcaaagcgcgaacattgtggtatgagatagcgatcgcccccaaaatctaa

SEQ ID NO: 38 - protein, 894 aa, Teredinibacter turnerae
MFLFISTHSPRLRLYRALLFLLLLFCVDALAEERLAESPIKSFTQTGLVPFVLPFDDNGAGITAFNNGSHQRGEGLAPLTIDSDGHFSVAGNRFRLWGVNITGDSAFPSHEDAEKIAGRLAKFGVNIVRFHHLDNNWGGAGLIDYRRGDSRHLSKDNLDKLDYFIAALKLRGIYSNINLLTAREFLPADELPASITQIDWKARQMLGAISPTVRNLEKAYAKKLLHHVNPYTRLAYRADPAIAFVEINNENSLFQQFFDGNIDRWPKEFKRPLAQEWNAWLTRKYKDQNALERAWQVIDKPLGNNLLTNANFVAGLQGWHLDQIDGAKAQASPLASAGLRIQVDTVGPALWNIQLSQNLPELKDGEIYTLSFAARSASHSQITPLVMQRAEPWQVVESFPVKLDSKWQEFRFRFVHSGSAQPLRLTLGELGSVIGAIDIRDLRLQPGGTVGELAANQTLERHSIGLNRNDESYLAQRREDWFAFLYSLELTYWQDMHRYLAEELKVKSNIYGTIASLSPPSIQREFGFIDSHIYWAHPHFPAGAWDAQQWSVDMSSMVNAFPNNTLSALARQRVAGLPFVVSEYQHAMPNPYSAEGPLLVAAYAGLQDWDGVYLFSYDQGELGWQQEFIDGFFKTNLNPAAMVNFAVGGNLFRRGDVQPAQGKRWLNFSPSRELAQIASAGASWSVSPANFPADWRGYAFHEQIGLQLEAPAAEPKLPVLDVNKVTADTGALTWDTSVQAQGRVTINTAKSAGVVGFIADQGFQLGALELRVGDLQMGWASWMITAQEGSLQDLAPGASLLAVATAKIENSKMRWNDAHNSLGRNWGEAPTRVEVVPFSLTLPVASRRVNAWCLDERGQRLHALKVEQTATGSRIDVDSKARTLWYEIAIAPKI

SEQ ID NO: 39 - DNA, 2604 bp, Unknown
  Microbial communities
atgatacggaattgttttttgccggctgcggcactaatgatcggttgcgcatttggggcggacgacgccatgttccccttcgtaccgtcgtacgacgcgccgatgaacgtggttaacatgagccatctgctggacgcgcccgccggcagccatgggcgcatccgtgttaaagacggacacttcgtgaacgaccagggccgggtcagactgcacgcgacgaatcttacgggaccggcgaactttccgtcgcacgaagaggcggagcgccttgcggcgcgtctggcccgtttcggcatcaactgcgtccgactgcactatttcgacagttcctacggcacgttcatgcttccggccgagcagggtatctttacggataaaaccggagaactccgacgtctcgatcccaagcagcgcgaccggcaggactatctgatcgcccagttcaagaaacgcggcatttatgtggatatcaacctgcacgtcgcccgtaccctggacgcaagcgacggtttcgagccgggaaccccatgggccaacaagggcgtggaccagtttgacccgcgggtgatcgcggcagaaaaagcgtatgcccgcgaattgctttcgcacgtcaatccctataccggactcagttacctgaaagaccccgttgtcgccatcgtggaactcaacaacgaggatgcgctgtggaatcaataccttaggggcgggattgaccagcttggaaaaccgtacgcgaaagaattccaacgccaatggaacgattggctgcgtaaaaaatacggcaacgacgaacgaatgcgcgcggcttggcgggtaaagacgcagccgctgggcgacgaaatgattcaggagggttcgtttgaagagaacgtggtgccggacggatcgcggtggattctcgaccttgagcgtacgaaagcgtctgccgcttcccaaaacggctcgctccgcatcacggtcgaaaagaagtcctccgaccgttttttccccaagctctaccgccgcgtatccgtaaaaaagaatgtcccatacacggtgtcgttccgaatccgccaagcggagggagagcctggcgaagtgggtttcgccgtggccgaccgcggcaagggctgggaatcgctgggcatccacaccgtccttaagcctaccaaaaactggacgaaacggaagttcacgttttatgccgcaaaggattgccagcaagcggaaatccagttcacgcgcttcgaggttggcgcttacgagattgacgacctttcgttccgaaccggcaacgaaccggaaaatctttccaccctttccgccgaaaagggcgaggtggcaatcgtgaagacgaaagactccgtagtgccggagatgaaacgcgacttctaccggtttgtaatggatacggaacacgcctattggacgggtatgcgcgattatctgcaaaaagagctcggactggaagcaccagtctccgccacccagctcgactattcgccgccgcatctgcaggcggagatggacttcgtggacaaccacgcctactggtgccacccgagcgtgcggaaagactggtcgatccgcaacaaggcgatggtaaacgcgcgcggcgggtgcattctcggcctcgcggggcaacgtgtggcgggcaaaccctacacggtaagcgagtacaaccacccctacccgatttactacggcgcggaggggcaacctatgctacgcgcctacggggcgttgcagggatgggacggcgtattcgaatattcctacaacaaccggcagaacgccgagccggaccacaacgagtatttcttcagcatggcggcacggaccgacgtgctggcgcatttcccggcatgcgcggcgatgtatctgcggggcgatgtcaaagagagcgcaacgcgtatcgtcgcgaatcttccgcttgaggaatatttcgaacggttggtgaaagccaacaaggtaagccaaggcatcaccacggcaaccgacgggaaactgtccgccgaactgggattggttcacagcgtggccgtggatgtgacggggcaaaccccgcgcacaacggataatattccgccgcccggcaagataatcgctagcgacacgggggaaatcgtttggaacaacgatatcgacggcgcgggcgtttggacggtggacacgccaaacacgaaaatcttctccggtttcccgaaggggcgcgtgttcaatctaagcggagtaaaactggcggttggagagactaaactcggttgggcgacggtgtcgctgacctcgcacgacgcgaccgggtttggcggggacggaaagcccgcgcgtattctgcttacggcgaccggactttctcacaacggcggagcaaagttcgtcgcaaagggaaaagaggcgatttttgcttccgagtggggcaacggtaaaacggtgaacgaaggtattcccgcgacagtcaccctacccgcgccgacggcaaagaccagctgctgggcacttgacgaacgcggtgaacggaaagcgaaagtacccgtgacggccgacgccgacggtcatgccgtcatcgcgatcggcccaacctggcaaacggtgtggtacgaaatcaacgttgagggatga

SEQ ID NO: 40 - protein, 867 aa, Unknown
  Microbial communities
MIRNCFLPAAALMIGCAFGADDAMFPFVPSYDAPMNVVNMSHLLDAPAGSHGRIRVKDGHFVNDQGRVRLHATNLTGPANFPSHEEAERLAARLARFGINCVRLHYFDSSYGTFMLPAEQGIFTDKTGELRRLDPKQRDRQDYLIAQFKKRGIYVDINLHVARTLDASDGFEPGTPWANKGVDQFDPRVIAAEKAYARELLSHVNPYTGLSYLKDPVVAIVELNNEDALWNQYLRGGIDQLGKPYAKEFQRQWNDWLRKKYGNDERMRAAWRVKTQPLGDEMIQEGSFEENVVPDGSRWILDLERTKASAASQNGSLRITVEKKSSDRFFPKLYRRVSVKKNVPYTVSFRIRQAEGEPGEVGFAVADRGKGWESLGIHTVLKPTKNWTKRKFTFYAAKDCQQAEIQFTRFEVGAYEIDDLSFRTGNEPENLSTLSAEKGEVAIVKTKDSVVPEMKRDFYRFVMDTEHAYWTGMRDYLQKELGLEAPVSATQLDYSPPHLQAEMDFVDNHAYWCHPSVRKDWSIRNKAMVNARGGCILGLAGQRVAGKPYTVSEYNHPYPIYYGAEGQPMLRAYGALQGWDGVFEYSYNNRQNAEPDHNEYFFSMAARTDVLAHFPACAAMYLRGDVKESATRIVANLPLEEYFERLVKANKVSQGITTATDGKLSAELGLVHSVAVDVTGQTPRTTDNIPPPGKIIASDTGEIVWNNDIDGAGVWTVDTPNTKIFSGFPKGRVFNLSGVKLAVGETKLGWATVSLTSHDATGFGGDGKPARILLTATGLSHNGGAKFVAKGKEAIFASEWGNGKTVNEGIPATVTLPAPTAKTSCWALDERGERKAKVPVTADADGHAVIAIGPTWQTVWYEINVEG

SEQ ID NO: 41 - DNA, 2622 bp, Planctomycetes
  CDS (1)..(2619)
  sig_peptide (1)..(81)
  mat_peptide (82)..(2619)
  PF00150 (277)..(687)
  PF02018 (850)..(1239)
atgaggcgaaacgttgcgttcgattgcattctgatcctgctacttgggctactgtgcttcggagcaacaccctctcggggagaagaaacggcaactccaggcaagctctttccgtttgtcctgagctacgaaccaacggacagcatcacaaacatctcagaatggcttgaccgtcccgctgggaagcacgggtttattcgggcggaaaatgggcactttgtgacagatgccgggcggatccggctgtgggccactaacctctgttttgaagcctgcttcccaaccaaggaagaggcagaacgccttgccaggcgtctcgccagcctggggatcaattgtgtgcgaatgcatcacatggacaatcggcacatctggggtaaaagccccaataagctgacgattgatcccgaaatgctggataagctggattacctgatttatcaattgaaattgcacgggatctataccaacctcaatctgcatgtgtcccgggagtttggcccggccgaaggctttcccgcggtggagggcctccccaactacgataaagggatcgacaactttgaaccccggatgatcgagtaccagaaaaaatatgcccgcgatttgctcacgcacgtcaatccctacaccggcacggcgtacatcaacgaaccggccattgcgatggtcgaaatcaataacgaaaatgcagcgtttgacgagtaccgcaagggagcgtttgatcatttgcccgagccgtacgccagccaactccgcaagctgtggaatgcctggctgaaaaagaaatacggcagtgacgacgcgcttcgcaaagcgtggaatgcccagcgtcaacccctgggcgaggaaatcctgaaaaatcgtgacttttccggccagtgggaaaaggtgtggaacctccagcgtgacaatctctcggaggtcgtcgccgaggtcattccgaatggctttcagggcaaacccgccttgcgtttgcgcgtcatccgcaacggacaagaaacctggatcccccagttaagccagggcggtttttcagttcagaaaggtcaggtgtacactctccgattctggctgaaagcggacaaaccgggccggatcgacgtgaactgcatgatgaaccacgatccctggcagcgtctcggcctttccgcggatgttcaaacctcggccgagtggaaggaatatcgcctcagctttgtggcggatcgcgatgatccaaatgccaggatcacgttcagccaactccgtcccgggacgtacgaactggcagacgtgtcactccggccgggtggggtcatcggcctggaagagggccaatccctcgccgatcagacggttcccattgttcctgctcgcggaccgcaaatgacggccgccgcccgggccgacttcgcagattttttgtgggagctcgaacgcgactactggtggggaatgtaccgatttctgaaggaggaactcaagctgaagccgctggtcgcgggaacgcaactctcctacagtccagttcacattcaagctgggctggactacatcgactcgcatgcctactggcagcatcccgttttccccggcaggccatgggatccggaaaactggtatgtgcgtagtctggccctcgtgaatcagccgggaggcacactttccggactcgccagtcggcgtgtcgaaggtttgccgttcaccgtgagcgaatacaaccacccggctcccaacgaatacgccgccgaaggatttccgatgatcgcggcttttggggcttttcaggattgggatggaatcttcagcttcacttacagccacagtcgagattacgagccgcgaaaaatcacgggtttcttcgacatcaaaagcgaggtgaccaaactcgttcacatgcccgcctgcgtcgccatgttctaccggggtgatgtgcaacccgccacccaggctgtggtcgtgggcatgacccgtgaaaaggaacaatccatcctccgagaaacactcaatccctgggcgctgaccgccgaccgtttgggtattcccgccaacctgagcttgctccatcgggtggccatggcactgaaagaacccagcgatagtgtgccaccacccacgctgtccgcggagcagaaggttttcctgtccgatacgcaacaaatctgctgggatgtctctcagcccggcgccggggtgttcctggtcaactcgccgaaaacgaaactcgtgaccggtttccccgccggaagaactttcaatctgaatggaatccagattcagattggagaaacggagctgggttgggcgaccgtttcgctcaccgttatcaaaggggacggatttgatcggcctggccgaatcctcctcgctgctacgggaaaggcccaaaatacaggctgggacttccgtaaagagggcgatcgggtgaccgtgggacgccgctggggcgacgagccgatcctctgcgaaggagtgccggctcgcatcgtgctgccggtttcgtccagccgcgtgaaagtctatgccctcgacgaggcgggacgccgcagggacgcggtgacggtttctggtggcgatcaggccgttgtcgaaatagggccccaattcaggacgctgtggtacgaaatcgaaatccaatga

SEQ ID NO: 42 - protein, 873 aa, Planctomycetes
MRRNVAFDCILILLLGLLCFGATPSRGEETATPGKLFPFVLSYEPTDSITNISEWLDRPAGKHGFIRAENGHFVTDAGRIRLWATNLCFEACFPTKEEAERLARRLASLGINCVRMHHMDNRHIWGKSPNKLTIDPEMLDKLDYLIYQLKLHGIYTNLNLHVSREFGPAEGFPAVEGLPNYDKGIDNFEPRMIEYQKKYARDLLTHVNPYTGTAYINEPAIAMVEINNENAAFDEYRKGAFDHLPEPYASQLRKLWNAWLKKKYGSDDALRKAWNAQRQPLGEEILKNRDFSGQWEKVWNLQRDNLSEVVAEVIPNGFQGKPALRLRVIRNGQETWIPQLSQGGFSVQKGQVYTLRFWLKADKPGRIDVNCMMNHDPWQRLGLSADVQTSAEWKEYRLSFVADRDDPNARITFSQLRPGTYELADVSLRPGGVIGLEEGQSLADQTVPIVPARGPQMTAAARADFADFLWELERDYWWGMYRFLKEELKLKPLVAGTQLSYSPVHIQAGLDYIDSHAYWQHPVFPGRPWDPENWYVRSLALVNQPGGTLSGLASRRVEGLPFTVSEYNHPAPNEYAAEGFPMIAAFGAFQDWDGIFSFTYSHSRDYEPRKITGFFDIKSEVTKLVHMPACVAMFYRGDVQPATQAVVVGMTREKEQSILRETLNPWALTADRLGIPANLSLLHRVAMALKEPSDSVPPPTLSAEQKVFLSDTQQICWDVSQPGAGVFLVNSPKTKLVTGFPAGRTFNLNGIQIQIGETELGWATVSLTVIKGDGFDRPGRILLAATGKAQNTGWDFRKEGDRVTVGRRWGDEPILCEGVPARIVLPVSSSRVKVYALDEAGRRRDAVTVSGGDQAVVEIGPQFRTLWYEIEIQ

(19) United States
(12) Patent Application Publication
(10) Pub. No.: US 2017/0175047 A1
(43) Pub. Date: Jun. 22, 2017

(54) Polypeptides Having Endoglucanase Activity

(71) Applicant: Novozymes A/S, Bagsvaerd (DK)

(72) Inventors: Kenneth Jensen, Oelsted (DK); Kirk M. Schnorr, Holte (DK); Jesper Solomon, Holte (DK); Bernard Henrissat, Marceille Cedex (FR); Leigh Murphy, Bagsvaerd (DK)

(73) Assignee: Novozymes A/S, Bagsvaerd (DK)

(21) Appl. No.: 15/312,208

(22) PCT Filed: May 28, 2015
(86) PCT No.: PCT/EP2015/061837

§ 371 Date: Nov. 18, 2016

(30) Foreign Application Priority Data
May 28, 2014 (EP) 14170186.2
May 28, 2014 (EP) 14170309.0
Aug. 19, 2014 (EP) 14181370.9

(51) Int. Cl. C11D 3/386; C09K 8/52; E21B 37/00; C12N 9/42; E21B 43/26; C09K 8/62; C12N 9/88

(52) U.S. Cl. CPC C11D 3/38636; C09K 8/62; C09K 8/52; C12N 9/88; C12N 9/2434; C12Y 402/02012; E21B 43/26; E21B 37/00; C09K 2208/24

(57) ABSTRACT

The present invention relates to endoglucanases having xanthan degrading activity and polynucleotides encoding the endoglucanases. The invention also relates to nucleic acid constructs, vectors, and host cells comprising the polynucleotides as well as methods of producing and using the endoglucanases.

### REFERENCE TO A SEQUENCE LISTING

This application contains a Sequence Listing in computer readable form, which is incorporated herein by reference.

### BACKGROUND OF THE INVENTION

Field of the Invention

The present invention relates to polypeptides having endoglucanase activity, in particular to polypeptides having endoglucanase activity and having activity on xanthan gum pretreated with xanthan lyase, and polynucleotides encoding the polypeptides. The invention also relates to nucleic acid constructs, vectors, and host cells comprising the polynucleotides as well as methods of producing and using the polypeptides. The invention further relates to compositions comprising the polypeptides and optionally xanthan lyases for use in detergents and in the drilling and oil industries.

Description of the Related Art

Xanthan gum is a polysaccharide derived from the bacterial coat of Xanthomonas campestris. It is produced by the fermentation of glucose, sucrose, or lactose by the Xanthomonas campestris bacterium. After a fermentation period, the polysaccharide is precipitated from a growth medium with isopropyl alcohol, dried, and ground into a fine powder. Later, the powder is added to a liquid medium to form the gum.

Xanthan is made up of pentasaccharide subunits, forming a cellulose backbone with trisaccharide side chains composed of mannose(β1,4)glucuronic-acid(β1,2)mannose attached to alternate glucose residues in the backbone by α1,3 linkages. This biopolymer is of great commercial significance because of its superior pseudoplasticity, thixotropy, and viscosity. Currently, it is widely used as a thickener or viscosifier in both food and nonfood industries and is used as a stabilizer for a wide variety of suspensions, emulsions, and foams.

In recent years xanthan gum has been use as an ingredient in many consumer products including foods (e.g. as thickening agent in salat dressings and dairy products) and cosmetics (e.g. as stabilizer and thickener in toothpaste and make-up to prevent ingredients from separating) and cosmetics (such as sun creams). Further xanthan gum has found use in the oil industry where xanthan gum is used in large quantities to thicken drilling mud. These fluids serve to carry the solids cut by the drilling bit back to the surface. When the circulation stops, the solids still remain suspended in the drilling fluid. The widespread use of horizontal drilling and the demand for good control of drilled solids has led to its expanded use. It is also added to self-consolidating concrete, including concrete poured underwater, to increase its viscosity.

The widespread use of xanthan gum has led to a desire to degrade and/or modify solutions or gels of xanthan gum. Complete enzymatic degradation of xanthan gum requires several enzymatic activities including xanthan lyase activity and endo-β-1,4-glucanase activity.

Xanthan lyases are enzymes that cleave the β-D-mannosyl-β-D-1,4-glucuronosyl bond of xanthan thereby removing the terminal pyruvated mannose. Two xanthan lyases been isolated from Paenibacillus alginolyticus XL-1 (e.g. Ruijssenaars et al. (1999) ‘A pyruvated mannose-specific xanthan lyase involved in xanthan degradation by Paenibacillus alginolyticus XL-1’, Appl. Environ. Microbiol. 65(6): 2446-2452, and Ruijssenaars et al. (2000), ‘A novel gene encoding xanthan lyase of Paenibacillus alginolyticus strain XL-1’, Appl. Environ. Microbiol. 66(9): 3945-3950).

The enzymes having endo-β-1,4-glucanase activity must be able to cut the highly substituted backbone of the xanthan gum after the removal of the terminal pyruvated mannose. Such enzymes are known from glycosyl hydrolase families GH9 (WO 2013/167581).

### SUMMARY OF THE INVENTION

The present inventors have surprisingly discovered a new group of enzymes that have endo-β-1,4-glucanase activity and are able to cut the highly substituted backbone of the xanthan gum—and which do not belong to the glycosyl hydrolase family known to comprise this enzymatic activity. The enzymes have no significant sequence similarity to any known enzyme having xanthan degrading activity and cannot be assigned to a known glycosyl hydrolase family. The new group of endoglucanases was identified by amino acid sequence alignment to the polypeptide disclosed in SEQ ID NO 2 of patent application no. EP14170186.2 and shown in SEQ ID NO 42 herein.

The invention provides new and improved enzymes for the degradation of xanthan gum and the use of such enzymes for cleaning purposes, such as the removal of xanthan gum stains, and in the drilling and oil industries. As the enzymes also has significant activity towards cellulose the enzymes may also be applied in a process for degradation of cellulosic material, e.g. in degradation of cellulosic biomass for production of e.g. fermentable sugars.

Accordingly, the present invention relates to polypeptides having endoglucanase activity and/or having activity on xanthan gum pretreated with xanthan lyase selected from the group consisting of:

(a) a polypeptide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40;

(b) a polypeptide encoded by a polynucleotide that hybridizes under medium stringency conditions with (i) the mature polypeptide coding sequence of any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, and SEQ ID NO: 39, (ii), or the full-length complement of (i);

(c) a polypeptide encoded by a polynucleotide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, and SEQ ID NO: 39;

(d) a variant of the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 comprising a substitution, deletion, and/or insertion at one or more positions; and

(e) a fragment of the polypeptide of (a), (b), (c), or (d) that has endoglucanase activity and/or has activity on xanthan gum pretreated with xanthan lyase.

The present invention also relates to polynucleotides encoding the polypeptides of the present invention; nucleic acid constructs; recombinant expression vectors; recombinant host cells comprising the polynucleotides; and methods of producing the polypeptides.

The present invention also relates to compositions comprising the polypeptides, and whole broth formulation or cell culture composition comprising the polypeptides.

The present invention also relates to use of the polypeptides and compositions for degrading xanthan gum, such as use in washing or cleaning a textile and/or a hard surface such as dish wash.

The present invention also relates to use of the polypeptides and compositions for degrading a cellulosic material.

### BRIEF DESCRIPTION OF THE FIGURES

FIG. 1 shows a multiple alignment of amino acid sequences SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40. Also included is SEQ ID NO: 42 which is identical to SEQ ID NO: 2 of patent application no. EP14170186.2.

### BRIEF DESCRIPTION OF THE SEQUENCES

SEQ ID Derived from
SEQ ID NO 01/02 Decomposing rain forest soil metagenome B
SEQ ID NO 03/04 Bioreactor enriched wastewater metagenome
SEQ ID NO 05/06 Lewinella cohaerens
SEQ ID NO 07/08 Chthoniobacter flavus
SEQ ID NO 09/10 Mesophilic rice straw/compost enrichment metagenome
SEQ ID NO 11/12 Human Stool microbiome
SEQ ID NO 13/14 Fervidibacteria bacterium
SEQ ID NO 15/16 Opitutaceae bacterium
SEQ ID NO 17/18 Opitutaceae bacterium
SEQ ID NO 19/20 Paludibacterium yongneupense
SEQ ID NO 21/22 Dickeya sp.
SEQ ID NO 23/24 Fervidibacteria bacterium
SEQ ID NO 25/26 Teredinibacter turnerae
SEQ ID NO 27/28 Teredinibacter turnerae
SEQ ID NO 29/30 Diplosphaera colitermitum
SEQ ID NO 31/32 Teredinibacter turnerae
SEQ ID NO 33/34 Teredinibacter turnerae
SEQ ID NO 35/36 Teredinibacter turnerae
SEQ ID NO 37/38 Teredinibacter turnerae
SEQ ID NO 39/40 Bovine rumen microbial communities
SEQ ID NO 41/42 Planctomycetes (SEQ ID NO: 2 of the patent application no. EP14170186.2)

### DEFINITIONS

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Cellulose binding domain: The term “cellulose binding domain” means the region of an enzyme that mediates binding of the enzyme to amorphous regions of a cellulose substrate.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Cleaning or Detergent Application: the term “cleaning or detergent application” means applying the polypeptide of the invention in any composition for the purpose of cleaning or washing, by hand, machine or automated, a hard surface or a textile.

Cleaning or Detergent Composition: the term “cleaning or detergent composition” refers to compositions that find use in the removal of undesired compounds from items to be cleaned, such as textiles, dishes, and hard surfaces. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; hard surface cleaning formulations, such as for glass, wood, ceramic and metal counter tops and windows; carpet cleaners; oven cleaners; fabric fresheners; fabric softeners; and textile and laundry pre-spotters, as well as dish wash detergents). In addition to the polypeptide of the invention, the detergent formulation may contain one or more additional enzymes (such as proteases, amylases, lipases, cutinases, cellulases, endoglucanases, xyloglucanases, pectinases, pectin lyases, xanthanases, xanthan lyases, peroxidaes, haloperoxygenases, catalases and mannanases, or any mixture thereof), and/or components such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxidoreductases, bluing agents and fluorescent dyes, antioxidants, and solubilizes.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Degrading xanthan gum: The term “degrading xanthan gum” or “xanthan degrading activity” is defined herein as the depolymerization, degradation or breaking down of xanthan gum into smaller components. The degradation of xanthan gum can either be the removal of one or more side chain saccharides, the cutting of the backbone of xanthan gum into smaller components or the removal of one or more side chain saccharides and the cutting of the backbone of xanthan gum into smaller components. The degradation of xanthan gum can preferably be measured using the viscosity reduction method as described in Example 5. Alternatively, the degradation of xanthan gum can be measured using the reducing ends method as described in Example 6.

Detergent Composition: the term “detergent composition” refers to compositions that find use in the removal of undesired compounds from items to be cleaned, such as textiles, dishes, and hard surfaces. The detergent composition may be used to e.g. clean textiles, dishes and hard surfaces for both household cleaning and industrial cleaning. The terms encompass any materials/compounds selected for the particular type of cleaning composition desired and the form of the product (e.g., liquid, gel, powder, granulate, paste, or spray compositions) and includes, but is not limited to, detergent compositions (e.g., liquid and/or solid laundry detergents and fine fabric detergents; hard surface cleaning formulations, such as for glass, wood, ceramic and metal counter tops and windows; carpet cleaners; oven cleaners; fabric fresheners; fabric softeners; and textile and laundry pre-spotters, as well as dish wash detergents). In addition to containing a polypeptide of the invention, the detergent formulation may contain one or more additional enzymes, such as xanthan lyases, proteases, amylases, lipases, cutinases, cellulases, endoglucanases, xyloglucanases, pectinases, pectin lyases, xanthanases, peroxidaes, haloperoxygenases, catalases and mannanases, or any mixture thereof), and/or components such as surfactants, builders, chelators or chelating agents, bleach system or bleach components, polymers, fabric conditioners, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

Dish wash: The term “dish wash” refers to all forms of washing dishes, e.g. by hand or automatic dish wash. Washing dishes includes, but is not limited to, the cleaning of all forms of crockery such as plates, cups, glasses, bowls, all forms of cutlery such as spoons, knives, forks and serving utensils as well as ceramics, plastics, metals, china, glass and acrylics.

Dish washing composition: The term “dish washing composition” refers to all forms of compositions for cleaning hard surfaces. The present invention is not restricted to any particular type of dish wash composition or any particular detergent.

Endoglucanase: The term “endoglucanase” means an endo-1,4-(1,3; 1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans, xyloglucans, xanthans and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481).

Endoglucanases having activity on xanthan gum pretreated with xanthan lyase: The term “endoglucanases having activity on xanthan gum pretreated with xanthan lyase” or “polypeptides having endoglucanase activity and/or having activity on xanthan gum pretreated with xanthan lyase” is defined as an endoglucanase having activity on xanthan gum pretreated with xanthan lyase. An endoglucanases of the invention has activity on xanthan gum pretreated with xanthan lyase. In one aspect of the invention a endoglucanases having activity on xanthan gum pretreated with xanthan lyase is a polypeptide having the sequence shown in any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40. Activity on xanthan gum pretreated with xanthan lyase can be determined as disclosed in Example 6.

Enzyme Detergency benefit: The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and or cleaning, prevention or reduction of redeposition of soils released in the washing process an effect that also is termed anti-redeposition, restoring fully or partly the whiteness of textiles, which originally were white but after repeated use and wash have obtained a greyish or yellowish appearance an effect that also is termed whitening. Textile care benefits, which are not directly related to catalytic stain removal or prevention of redeposition of soils are also important for enzyme detergency benefits. Examples of such textile care benefits are prevention or reduction of dye transfer from one fabric to another fabric or another part of the same fabric an effect that is also termed dye transfer inhibition or anti-backstaining, removal of protruding or broken fibers from a fabric surface to decrease pilling tendencies or remove already existing pills or fuzz an effect that also is termed anti-pilling, improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching component such as hydrogen peroxide or other peroxides.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide having one or more (e.g., several) amino acids absent from the amino and/or carboxyl terminus of a mature polypeptide; wherein the fragment has endoglucanase activity and/or has activity on xanthan gum pretreated with xanthan lyase.

Glycosyl hydrolase families: Glycoside hydrolases are enzymes that catalyse the hydrolysis of the glycosyl bond to release smaller sugars. There are over 100 classes of glycoside hydrolases which have been classified into glycosyl hydrolase (GH) families, see Henrissat et al. (1991) ‘A classification of glycosyl hydrolases based on amino-acid sequence similarities’, J. Biochem. 280: 309-316 and the Uniprot website at www.cazy.org.

Hard surface cleaning: The term “Hard surface cleaning” is defined herein as cleaning of hard surfaces wherein hard surfaces may include floors, tables, walls, roofs etc. as well as surfaces of hard objects such as cars (car wash) and dishes (dish wash). Dish washing includes but are not limited to cleaning of plates, cups, glasses, bowls, and cutlery such as spoons, knives, forks, serving utensils, ceramics, plastics, metals, china, glass and acrylics.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Improved wash performance: The term “improved wash performance” is defined herein as a (variant) enzyme (also a blend of enzymes, not necessarily only variants but also backbones, and in combination with certain cleaning composition etc.) displaying an alteration of the wash performance of a protease variant relative to the wash performance of the parent protease variant e.g. by increased stain removal. The term “wash performance” includes wash performance in laundry but also e.g. in dish wash.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). An isolated substance may be present in a fermentation broth sample; e.g. a host cell may be genetically modified to express the polypeptide of the invention. The fermentation broth from that host cell will comprise the isolated polypeptide.

Laundering: The term “laundering” relates to both household laundering and industrial laundering and means the process of treating textiles with a solution containing a cleaning or detergent composition of the present invention. The laundering process can for example be carried out using e.g. a household or an industrial washing machine or can be carried out by hand.

Mature polypeptide: The term “mature polypeptide” means a polypeptide in its final form following translation and any post-translational modifications, such as N-terminal processing, C-terminal truncation, glycosylation, phosphorylation, etc. The mature peptide of the endo-glucanases are: positions 1 to 813 in SEQ ID NO: 2, positions 1 to 836 in SEQ ID NO: 4, positions 1 to 949 in SEQ ID NO: 6, positions 1 to 830 in SEQ ID NO: 8, positions 1 to 866 in SEQ ID NO: 10, positions 1 to 827 in SEQ ID NO: 12, positions 1 to 903 in SEQ ID NO: 14, positions 1 to 932 in SEQ ID NO: 16, positions 1 to 920 in SEQ ID NO:18, positions 1 to 844 in SEQ ID NO:20, positions 1 to 849 in SEQ ID NO:22, positions 1 to 903 in SEQ ID NO:24, positions 1 to 894 in SEQ ID NO:26, positions 1 to 894 in SEQ ID NO:28, positions 1 to 955 in SEQ ID NO:30, positions 1 to 894 in SEQ ID NO:32, positions 1 to 893 in SEQ ID NO:34, positions 1 to 894 in SEQ ID NO:36, positions 1 to 894 in SEQ ID NO:38, and positions 1 to 867 in SEQ ID NO:40.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having endoglucanase activity and/or having activity on xanthan gum pretreated with xanthan lyase. The mature peptide encoding part of the endo-glucanase genes are: positions 67 to 2508 in SEQ ID NO: 1, positions 76 to 2583 in SEQ ID NO: 3, positions 67 to 2913 in SEQ ID NO: 5, positions 73 to 2562 in SEQ ID NO: 7, positions 115 to 2712 in SEQ ID NO: 9, positions 103 to 2565 in SEQ ID NO: 11, positions 1 to 2712 in SEQ ID NO: 13, positions 1 to 2799 in SEQ ID NO: 15, positions 1 to 2763 in SEQ ID NO:17, positions 1 to 2535 in SEQ ID NO:19, positions 1 to 2550 in SEQ ID NO:21, positions 1 to 2712 in SEQ ID NO:23, positions 1 to 2685 in SEQ ID NO:25, positions 1 to 2685 in SEQ ID NO:27, positions 1 to 2868 in SEQ ID NO:29, positions 1 to 2685 in SEQ ID NO:31, positions 1 to 2682 in SEQ ID NO:33, positions 1 to 2685 in SEQ ID NO:35, positions 1 to 2685 in SEQ ID NO:37, and positions 1 to 2604 in SEQ ID NO:39.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides×100)/(Length of Alignment−Total Number of Gaps in Alignment)

Stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

Subsequence: The term “subsequence” means a polynucleotide having one or more (e.g., several) nucleotides absent from the 5′ and/or 3′ end of a mature polypeptide coding sequence; wherein the subsequence encodes a fragment having endoglucanase activity and/or having activity on xanthan gum pretreated with xanthan lyase.

Textile: The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir or manmade cellulosics (e.g. originating from wood pulp) including viscose/rayon, ramie, cellulose acetate fibers (tricell), lyocell or blends thereof. The textile or fabric may also be non-cellulose based such as natural polyamides including wool, camel, cashmere, mohair, rabit and silk or synthetic polymer such as nylon, aramid, polyester, acrylic, polypropylen and spandex/elastane, or blends thereof as well as blend of cellulose based and non-cellulose based fibers. Examples of blends are blends of cotton and/or rayon/viscose with one or more companion material such as wool, synthetic fibers (e.g. polyamide fibers, acrylic fibers, polyester fibers, polyvinyl alcohol fibers, polyvinyl chloride fibers, polyurethane fibers, polyurea fibers, aramid fibers), and cellulose-containing fibers (e.g. rayon/viscose, ramie, flax/linen, jute, cellulose acetate fibers, lyocell). Fabric may be conventional washable laundry, for example stained household laundry. When the term fabric or garment is used it is intended to include the broader term textiles as well.

Textile care benefit: “Textile care benefits”, which are not directly related to catalytic stain removal or prevention of redeposition of soils, are also important for enzyme detergency benefits. Examples of such textile care benefits are prevention or reduction of dye transfer from one textile to another textile or another part of the same textile an effect that is also termed dye transfer inhibition or anti-backstaining, removal of protruding or broken fibers from a textile surface to decrease pilling tendencies or remove already existing pills or fuzz an effect that also is termed anti-pilling, improvement of the textile-softness, colour clarification of the textile and removal of particulate soils which are trapped in the fibers of the textile. Enzymatic bleaching is a further enzyme detergency benefit where the catalytic activity generally is used to catalyze the formation of bleaching component such as hydrogen peroxide or other peroxides or other bleaching species.

Variant: The term “variant” means a polypeptide having endoglucanase activity and/or activity on xanthan gum pretreated with xanthan lyase, and comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

Wash performance: The term “wash performance” is used as an enzyme's ability to remove stains present on the object to be cleaned during e.g. wash or hard surface cleaning. The improvement in the wash performance may be quantified by calculating the so-called intensity value (Int) as in the ‘Automatic Mechanical Stress Assay (AMSA) for laundry’ or the remission value (Rem) as defined in WO 2013/167581.

Whiteness: The term “Whiteness” is defined herein as a broad term with different meanings in different regions and for different customers. Loss of whiteness can e.g. be due to greying, yellowing, or removal of optical brighteners/hueing agents. Greying and yellowing can be due to soil redeposition, body soils, colouring from e.g. iron and copper ions or dye transfer. Whiteness might include one or several issues from the list below: Colorant or dye effects; Incomplete stain removal (e.g. body soils, sebum ect.); Re-deposition (greying, yellowing or other discolorations of the object) (removed soils re-associates with other part of textile, soiled or unsoiled); Chemical changes in textile during application; and Clarification or brightening of colours.

Xanthan Lyase: The term “xanthan lyase” is defined herein as an enzyme that cleaves the β-D-mannosyl-β-D-1,4-glucuronosyl bonds in xanthan gum (EC 4.2.2.12). For purposes of the present invention, xanthan lyase activity is determined according to the procedure described in Example 6.

### DETAILED DESCRIPTION OF THE INVENTION

The present invention provides endoglucanases having activity on xanthan gum pretreated with xanthan lyase and polynucleotides encoding the polypeptides. The endoglucanase do not belong to a GH family known to comprise enzymes which degrade xanthan. In addition, the combination of xanthan lyase and an endoglucanase of the invention having activity on xanthan gum pretreated with xanthan lyase of the invention shows a synergistic improved wash performance over using xanthan lyase or endoglucanases having activity on xanthan gum pretreated with xanthan lyase alone. In addition the enzyme may have activity towards any of the substrates cellulose, curdlan, and β-glucan.

Endoglucanases Having Activity on Xanthan Gum Pretreated with Xanthan Lyase

In an embodiment, the present invention relates to polypeptides having a sequence identity to the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have endoglucanase which have and activity on xanthan gum pretreated with xanthan lyase. In one aspect, the polypeptides differ by no more than 50 amino acids, e.g., 1,2, 3, 4, 5, 6, 7, 8, 9, 10, 11, 12, 13, 14, 15, 16, 17, 18, 19, 20, 21,22, 23, 24, 25, 26, 27, 28, 29, 30, 31, 32, 33, 34, 35, 36, 37, 38, 39, 40, 41,42, 43, 44, 45, 46, 47, 48, or 49, from the mature polypeptide of SEQ ID NO: 14. In a preferred aspect, the polypeptides differ by up to 10 amino acids, e.g., 1, 2, 3, 4, 5, 6, 7, 8, 9, or 10, from the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40.

In a particular embodiment the invention relates to polypeptides having a sequence identity to the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 of at least 60%, at least 65%, at least 75%, at least 70%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the endoglucanase activity and/or xanthan degrading activity of the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40.

In a particular embodiment the invention relates to polypeptides having a sequence identity to the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 of at least 60%, at least 65%, at least 75%, at least 70%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the endoglucanase activity and/or xanthan degrading activity of the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40.

In a particular embodiment the invention relates to polypeptides having a sequence identity to the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 of at least 60%, at least 65%, at least 75%, at least 70%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the endoglucanase activity and/or xanthan degrading activity of the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40.

In a particular embodiment the invention relates to polypeptides having a sequence identity to the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 of at least 60%, at least 65%, at least 75%, at least 70%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the endoglucanase activity and/or xanthan degrading activity of the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40.

In a particular embodiment the invention relates to polypeptides having a sequence identity to the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 of at least 60%, at least 65%, at least 75%, at least 70%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the endoglucanase activity and/or xanthan degrading activity of the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40.

In a particular embodiment the invention relates to polypeptides having a sequence identity to the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 of at least 60%, at least 65%, at least 75%, at least 70%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the endoglucanase activity and/or xanthan degrading activity of the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40.

In a particular embodiment the invention relates to polypeptides having a sequence identity to the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 of at least 60%, at least 65%, at least 75%, at least 70%, at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the endoglucanase activity and/or xanthan degrading activity of the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40.

In an embodiment, the polypeptide has been isolated.

A polypeptide of the present invention preferably comprises or consists of the amino acids 1 to 846 of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 or an allelic variant thereof; or is a fragment thereof having endoglucanase activity and/or xanthan degrading activity. In another aspect, the polypeptide comprises or consists of the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40.

In another embodiment, the present invention relates to a polypeptide having endoglucanase activity and/or activity on xanthan gum pretreated with xanthan lyase which is encoded by a polynucleotide that hybridizes under high stringency conditions with (i) the mature polypeptide coding sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, and SEQ ID NO: 39, or (ii) the full-length complement of (i) (Sambrook et al., 1989, Molecular Cloning, A Laboratory Manual, 2d edition, Cold Spring Harbor, New York). In an embodiment, the polypeptide has been isolated.

The polynucleotide of SEQ ID NO: 1 or a subsequence thereof, as well as the polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 or a fragment thereof may be used to design nucleic acid probes to identify and clone DNA encoding polypeptides having endoglucanase activity and/or activity on xanthan gum pretreated with xanthan lyase according to methods well known in the art. In particular, such probes can be used for hybridization with the genomic DNA or cDNA of a cell of interest from strains of different genera or species, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide of the invention. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, and SEQ ID NO: 39, or a subsequence thereof, the carrier material is used in a Southern blot.

For purposes of the present invention, hybridization indicates that the polynucleotide hybridizes to a labeled nucleic acid probe corresponding to (i) any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, and SEQ ID NO: 39; (ii) the mature polypeptide coding sequence of any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, and SEQ ID NO: 39; (iii) the full-length complement thereof; or (iv) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is nucleotides is a subsequence of any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, and SEQ ID NO: 39. In another aspect, the nucleic acid probe is a polynucleotide that encodes the polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40; the mature polypeptide thereof; or a fragment thereof. In another aspect, the nucleic acid probe is any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, and SEQ ID NO: 39.

In another embodiment, the present invention relates to an polypeptide having endoglucanase activity and/or having activity on xanthan gum pretreated with xanthan lyase encoded by a polynucleotide having a sequence identity to the mature polypeptide coding sequence of any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, and SEQ ID NO: 39, of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 comprising a substitution, deletion, and/or insertion at one or more (e.g., several) positions. In an embodiment, the number of amino acid substitutions, deletions and/or insertions introduced into the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 is up to 10, e.g., 1,2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a His-tag (poly-histidine tract), an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, AlaN/al, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for endoglucanase activity and/or xanthan degrading activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Sources of Polypeptides Having Endoglucanase Activity and/or Activity on Xanthan Gum Pretreated with Xanthan Lyase

A polypeptide of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

In a aspect, the polypeptide is a bacterial polypeptide. In another aspect, the polypeptide is a fungal polypeptide. In a aspect, the polypeptide is a polypeptide obtained from any of Lewinella sp., Chthoniobacter sp, Fervidibacteria sp., Opitutaceae sp., Dickeya sp., Teredinibacter sp., and Diplosphaera sp., including the species Lewinella cohaerens, Chthoniobacter flavus, Teredinibacter turnerae, and Diplosphaera colitermitum.

Strains of this and related species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to polynucleotides encoding a polypeptide of the present invention, as described herein. In an embodiment, the polynucleotide encoding the polypeptide of the present invention has been isolated.

The techniques used to isolate or clone a polynucleotide are known in the art and include isolation from genomic DNA or cDNA, or a combination thereof. The cloning of the polynucleotides from genomic DNA can be effected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Lewinella sp., Chthoniobacter sp, Fervidibacteria sp., Opitutaceae sp., Dickeya sp., Teredinibacter sp., and Diplosphaera sp., including the species Lewinella cohaerens, Chthoniobacter flavus, Teredinibacter turnerae, and Diplosphaera colitermitum, or a related organism and thus, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence of any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, and SEQ ID NO: 39, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xyIA and xyIB genes, Bacillus thuringiensis cryIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide. In one aspect, the cell is a Planctomycete cell. In another aspect, the cell is a cell from the Planctomycete sp. R1 strain.

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Plants

The present invention also relates to isolated plants, e.g., a transgenic plant, plant part, or plant cell, comprising a polynucleotide of the present invention so as to express and produce a polypeptide in recoverable quantities. The polypeptide may be recovered from the plant or plant part. Alternatively, the plant or plant part containing the polypeptide may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilization of the invention are also considered plant parts, e.g., embryos, endosperms, aleurone and seed coats.

Also included within the scope of the present invention are the progeny of such plants, plant parts, and plant cells.

The transgenic plant or plant cell expressing the polypeptide may be constructed in accordance with methods known in the art.

The present invention also relates to methods of producing a polypeptide of the present invention comprising (a) cultivating a transgenic plant or a plant cell comprising a polynucleotide encoding the polypeptide under conditions conducive for production of the polypeptide; and (b) recovering the polypeptide.

### Fermentation Broth Formulations or Cell Compositions

The present invention also relates to a fermentation broth formulation or a cell composition comprising a polypeptide of the present invention. The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Detergent Compositions

In an embodiment, the present invention relates to a detergent composition comprising isolated endoglucanases having activity on xanthan gum pretreated with xanthan lyase and having a sequence identity to the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 of at least 60%, e.g., at least 65%, at least 70%, at least 70%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In one embodiment, the invention is directed to detergent compositions comprising an enzyme of the present invention in combination with one or more additional cleaning composition components. The choice of additional components is within the skill of the artisan and includes conventional ingredients, including the exemplary non-limiting components set forth below.

The choice of components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation since the component may have one or more additional functionalities which the skilled artisan will appreciate.

The detergent composition may be suitable for the laundering of textiles such as e.g. fabrics, cloths or linen, or for cleaning hard surfaces such as e.g. floors, tables, or dish wash.

Enzyme of the Present Invention—

In one embodiment of the present invention, the a polypeptide of the present invention may be added to a detergent composition in an amount corresponding to 0.0001-200 mg of enzyme protein, such as 0.0005-100 mg of enzyme protein, preferably 0.001-30 mg of enzyme protein, more preferably 0.005-8 mg of enzyme protein, even more preferably 0.01-2 mg of enzyme protein per litre of wash liquor.

A composition for use in automatic dishwash (ADW), for example, may include 0.0001%-50%, such as 0.001%-20%, such as 0.01%-10%, such as 0.05-5% of enzyme protein by weight of the composition.

A composition for use in laundry granulation, for example, may include 0.0001%-50%, such as 0.001%-20%, such as 0.01%-10%, such as 0.05%-5% of enzyme protein by weight of the composition.

A composition for use in laundry liquid, for example, may include 0.0001%-10%, such as 0.001-7%, such as 0.1%-5% of enzyme protein by weight of the composition.

The enzyme(s) of the detergent composition of the invention may be stabilized using conventional stabilizing agents, e.g., a polyol such as propylene glycol or glycerol, a sugar or sugar alcohol, lactic acid, boric acid, or a boric acid derivative, e.g., an aromatic borate ester, or a phenyl boronic acid derivative such as 4-formylphenyl boronic acid, and the composition may be formulated as described in, for example, WO92/19709 and WO92/19708.

In certain markets different wash conditions and, as such, different types of detergents are used. This is disclosed in e.g. EP 1 025 240. For example, In Asia (Japan) a low detergent concentration system is used, while the United States uses a medium detergent concentration system, and Europe uses a high detergent concentration system.

A low detergent concentration system includes detergents where less than about 800 ppm of detergent components are present in the wash water. Japanese detergents are typically considered low detergent concentration system as they have approximately 667 ppm of detergent components present in the wash water.

A medium detergent concentration includes detergents where between about 800 ppm and about 2000 ppm of detergent components are present in the wash water. North American detergents are generally considered to be medium detergent concentration systems as they have approximately 975 ppm of detergent components present in the wash water.

A high detergent concentration system includes detergents where greater than about 2000 ppm of detergent components are present in the wash water. European detergents are generally considered to be high detergent concentration systems as they have approximately 4500-5000 ppm of detergent components in the wash water.

Latin American detergents are generally high suds phosphate builder detergents and the range of detergents used in Latin America can fall in both the medium and high detergent concentrations as they range from 1500 ppm to 6000 ppm of detergent components in the wash water. Such detergent compositions are all embodiments of the invention.

A polypeptide of the present invention may also be incorporated in the detergent formulations disclosed in WO97/07202, which is hereby incorporated by reference.

Surfactants—

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and includes any conventional surfactant(s) known in the art. Any surfactant known in the art for use in detergents may be utilized.

When included therein the detergent will usually contain from about 1% to about 40% by weight, such as from about 5% to about 30%, including from about 5% to about 15%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein the detergent will usually contain from about 0% to about 10% by weight of a cationic surfactant. Non-limiting examples of cationic surfactants include alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

When included therein the detergent will usually contain from about 0.2% to about 40% by weight of a non-ionic surfactant, for example from about 0.5% to about 30%, in particular from about 1% to about 20%, from about 3% to about 10%, such as from about 3% to about 5%, or from about 8% to about 12%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein the detergent will usually contain from about 0% to about 10% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, and combinations thereof.

When included therein the detergent will usually contain from about 0% to about 10% by weight of a zwitterionic surfactant. Non-limiting examples of zwitterionic surfactants include betaine, alkyldimethylbetaine, sulfobetaine, and combinations thereof.

Hydrotropes—

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-5% by weight, such as about 0.5 to about 5%, or about 3% to about 5%, of a hydrotrope. Any hydrotrope known in the art for use in detergents may be utilized. Non-limiting examples of hydrotropes include sodium benzene sulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

Builders and Co-Builders—

The detergent composition may contain about 0-65% by weight, such as about 5% to about 45% of a detergent builder or co-builder, or a mixture thereof. In a dish wash deteregent, the level of builder is typically 40-65%, particularly 50-65%. The builder and/or co-builder may particularly be a chelating agent that forms water-soluble complexes with Ca and Mg. Any builder and/or co-builder known in the art for use in laundry detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as iminodiethanol), triethanolamine (TEA, also known as 2,2′,2″-nitrilotriethanol), and carboxymethyl inulin (CMI), and combinations thereof.

The detergent composition may also contain 0-20% by weight, such as about 5% to about 10%, of a detergent co-builder, or a mixture thereof. The detergent composition may include include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra-(methylenephosphonic acid) (EDTMPA), diethylenetriaminepentakis(methylenephosphonic acid) (DTPMPA or DTMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N, N-diacetic acid (α-ALDA), serine-N, N-diacetic acid (SEDA), isoserine-N, N-diacetic acid (ISDA), phenylalanine-N, N-diacetic acid (PHDA), anthranilic acid-N, N-diacetic acid (ANDA), sulfanilic acid-N, N-diacetic acid (SLDA), taurine-N, N-diacetic acid (TUDA) and sulfomethyl-N, N-diacetic acid (SMDA), N-(2-hydroxyethyl)-ethylidenediamine-N, N′,N′-triacetate (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

Bleaching Systems—

The detergent may contain 0-50% by weight, such as about 0.1% to about 25%, of a bleaching system. Any bleaching system known in the art for use in laundry detergents may be utilized. Suitable bleaching system components include bleaching catalysts, photobleaches, bleach activators, sources of hydrogen peroxide such as sodium percarbonate and sodium perborates, preformed peracids and mixtures thereof. Suitable preformed peracids include, but are not limited to, peroxycarboxylic acids and salts, percarbonic acids and salts, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with peroxygen bleach like hydrogen peroxide to form a peracid. The peracid thus formed constitutes the activated bleach. Suitable bleach activators to be used herein include those belonging to the class of esters amides, imides or anhydrides. Suitable examples are tetracetylethylene diamine (TAED), sodium 4-[(3,5,5-trimethylhexanoyl)oxy]benzene sulfonate (ISONOBS), diperoxy dodecanoic acid, 4-(dodecanoyloxy)benzenesulfonate (LOBS), 4-(decanoyloxy)benzenesulfonate, 4-(decanoyloxy)benzoate (DOBS), 4-(nonanoyloxy)-benzenesulfonate (NOBS), and/or those disclosed in WO98/17767. A particular family of bleach activators of interest was disclosed in EP624154 and particulary preferred in that family is acetyl triethyl citrate (ATC). ATC or a short chain triglyceride like triacetin has the advantage that it is environmental friendly as it eventually degrades into citric acid and alcohol. Furthermore acetyl triethyl citrate and triacetin has a good hydrolytical stability in the product upon storage and it is an efficient bleach activator. Finally ATC provides a good building capacity to the laundry additive. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

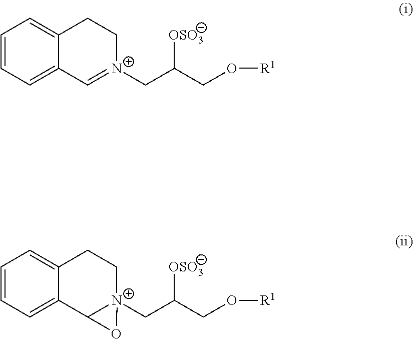

(iii) and mixtures thereof; wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl. Other exemplary bleaching systems are described, e.g. in WO2007/087258, WO2007/087244, WO2007/087259 and WO2007/087242. Suitable photobleaches may for example be sulfonated zinc phthalocyanine.

Polymers—

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

Fabric Hueing Agents—

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % fabric hueing agent. The composition may comprise from 0.0001 wt % to 0.2 wt % fabric hueing agent, this may be especially preferred when the composition is in the form of a unit dose pouch. Suitable hueing agents are also disclosed in, e.g. WO 2007/087257 and WO2007/087243.

Additional Enzymes—

The detergent additive as well as the detergent composition may comprise one or more additional enzymes such as a protease, lipase, cutinase, an amylase, carbohydrase, cellulase, pectinase, mannanase, arabinase, galactanase, xylanase, oxidase, e.g., a laccase, peroxidase and/or xanthan lyase.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

Cellulases:

Suitable cellulases include those of bacterial or fungal origin. Chemically modified or protein engineered variants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having color care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. Pat. No. 5,686,593, U.S. Pat. No. 5,763,254, WO 95/24471, WO 98/12307 and PCT/DK98/00299.

Example of cellulases exhibiting endo-beta-1,4-glucanase activity (EC 3.2.1.4) are those having described in WO02/099091.

Other examples of cellulases include the family 45 cellulases described in WO96/29397, and especially variants thereof having substitution, insertion and/or deletion at one or more of the positions corresponding to the following positions in SEQ ID NO: 8 of WO 02/099091: 2, 4, 7, 8, 10, 13, 15, 19, 20, 21,25, 26, 29, 32, 33, 34, 35, 37, 40, 42, 42a, 43, 44, 48, 53, 54, 55, 58, 59, 63, 64, 65, 66, 67, 70, 72, 76, 79, 80, 82, 84, 86, 88, 90, 91, 93, 95, 95d, 95h, 95j, 97, 100, 101, 102, 103, 113, 114, 117, 119, 121, 133, 136, 137, 138, 139, 140a, 141, 143a, 145, 146, 147, 150e, 150j, 151, 152, 153, 154, 155, 156, 157, 158, 159, 160c, 160e, 160k, 161,162, 164, 165, 168, 170, 171,172, 173, 175, 176, 178, 181, 183, 184, 185, 186, 188, 191, 192, 195, 196, 200, and/or 20, preferably selected among P19A, G20K, Q44K, N48E, Q119H or Q146 R.

Commercially available cellulases include Celluzyme™, and Carezyme™ (Novozymes A/S), Clazinase™, and Puradax HA™ (Genencor International Inc.), and KAC-500(B)™ (Kao Corporation).

Proteases:

The additional enzyme may be another protease or protease variant. The protease may be of animal, vegetable or microbial origin, including chemically or genetically modified variants. Microbial origin is preferred. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4, M5, M7 or M8.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family. In one aspect of the invention the protease may be a subtilase, such as a subtilisin or a variant hereof. Further the subtilases (and the serine proteases) are characterised by having two active site amino acid residues apart from the serine, namely a histidine and an aspartic acid residue.

Examples of subtilisins are those derived from Bacillus such as subtilisin lentus, Bacillus lentus, subtilisin Novo, subtilisin Carlsberg, Bacillus licheniformis, subtilisin BPN′, subtilisin 309, subtilisin 147 and subtilisin 168 described in WO 89/06279 and protease PD138 (WO 93/18140). Additional serine protease examples are described in WO 98/020115, WO 01/44452, WO 01/58275, WO 01/58276, WO 03/006602 and WO 04/099401. An example of a subtilase variants may be those having mutations in any of the positions: 3, 4, 9, 15, 27, 36, 68, 76, 87, 95, 96, 97, 98, 99, 100, 101, 102, 103, 104, 106, 118, 120, 123, 128, 129, 130, 160, 167, 170, 194, 195, 199, 205, 217, 218, 222, 232, 235, 236, 245, 248, 252 and 274 using the BPN′-numbering. More preferred the subtilase variants may comprise the mutations: S3T, V4I, S9R, A15T, K27R, *36D, V68A, N76D, N87S,R, *97E, A98S, S99G,D,A, S99AD, S101G,M,R S103A, V104I,Y,N, S106A, G118V,R, H120D,N, N123S, S128L, P129Q, S130A, G160D, Y167A, R170S, A194P, G195E, V199M, V2051, L217D, N218D, M222S, A232V, K235L, Q236H, Q245R, N252K, T274A (using BPN′ numbering). A further preferred protease is the alkaline protease from Bacillus lentus DSM 5483, as described for example in WO 95/23221, and variants thereof which are described in WO 92/21760, WO 95/23221, EP 1921147 and EP 1921148.

Examples of trypsin-like proteases are trypsin (e.g. of porcine or bovine origin) and the Fusarium protease described in WO 89/06270 and WO 94/25583. Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235, and 274.

Examples of metalloproteases are the neutral metalloprotease as described in WO 07/044993.

Preferred commercially available protease enzymes include Alcalase™, Coronase™ Duralase™, Durazym™, Esperase™, Everlase™, Kannase™, Liquanase™, Liquanase Ultra™, Ovozyme™, Polarzyme™, Primase™, Relase™, Savinase™ and Savinase Ultra™, (Novozymes A/S), Axapem™ (Gist-Brocases N.V.), BLAP and BLAP X (Henkel AG & Co. KGaA), Excellase™, FN2™, FN3™, FN4™, Maxaca™, Maxapem™, Maxatase™, Properase™ Purafast™, Purafect™, Purafect OxP™, Purafect Prime™ and Puramax™ (Genencor int.).

Lipases and Cutinases:

Suitable lipases and cutinases include those of bacterial or fungal origin. Chemically modified or protein engineered variant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147).

Further examples are lipases sometimes referred to as acyltransferases or perhydrolases, e.g. acyltransferases with homology to Candida antarctica lipase A (WO 10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Amylases—

The amylase may be an alpha-amylase, a beta-amylase or a glucoamylase and may be of bacterial or fungal origin. Chemically modified or protein engineered variants are included. Amylases include, for example, alpha-amylases obtained from Bacillus, e.g., a special strain of Bacillus licheniformis, described in more detail in GB 1,296,839.

Examples of amylases are those having SEQ ID NO: 3 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211,243, 264, 304, 305, 391, 408, and 444 of SEQ ID NO: 3 in WO 95/10603.

Further amylases which can be used are amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylase examples are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48+T49+G107+H156+A181+N190+I201+A209+Q264.

Further amylase examples are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions G182 and H183 or positions H183 and G184.

Additional amylases are those having SEQ ID NO: 1, SEQ ID NO: 2 or SEQ ID NO: 7 of WO 96/023873 or variants thereof having 90% sequence identity to SEQ ID NO: 1, SEQ ID NO: 2 or SEQ ID NO: 7. Preferred variants of SEQ ID NO: 1, SEQ ID NO: 2 or SEQ ID NO: 7 are those having a substitution, a deletion or an insertion in one or more of the following positions: 140, 181, 182, 183, 184, 195, 206, 212, 243, 260, 269, 304 and 476. More preferred variants are those having a deletion in positions 182 and 183 or positions 183 and 184. Most preferred amylase variants of SEQ ID NO: 1, SEQ ID NO: 2 or SEQ ID NO: 7 are those having a deletion in positions 183 and 184 and a substitution in positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further amylases which can be used are amylases having SEQ ID NO: 2 of WO 09/061380 or variants thereof having 90% sequence identity to SEQ ID NO: 2. Preferred variants of SEQ ID NO: 2 are those having a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K wherein the variant optionally further comprises a substitution at position 243 and/or a deletion at position 180 and/or position 181.

Other examples of amylases are the alpha-amylase having SEQ ID NO: 12 in WO01/66712 or a variant having at least 90%, such as at least 95%, sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Commercially available amylases are Duramyl™, Termamyl™, Fungamyl™ Stainzyme™, Stainzyme Plus™, Natalase™ and BAN™ (Novozymes A/S), Rapidase™ and Purastar™ (from Genencor International Inc.).

Peroxidases/Oxidases:

Suitable peroxidases/oxidases include those of plant, bacterial or fungal origin. Chemically modified or protein engineered variants are included. Examples of useful peroxidases include peroxidases from Coprinus, e.g., from C. cinereus, and variants thereof as those described in WO 93/24618, WO 95/10602, and WO 98/15257.

Commercially available peroxidases include Guardzyme™ (Novozymes A/S).

The enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as a granulate, liquid, slurry, etc. Preferred detergent additive formulations are granulates, in particular non-dusting granulates, liquids, in particular stabilized liquids, or slurries.

Non-dusting granulates may be produced, e.g., as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

Adjunct Materials—

Any detergent components known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

Dispersants:

The detergent compositions of the present invention can also contain dispersants. In particular powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

Dye Transfer Inhibiting Agents:

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent Whitening Agent:

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulphonic acid derivatives, diarylpyrazoline derivatives and bisphenyl-distyryl derivatives. Examples of the diaminostilbene-sulphonic acid derivative type of fluorescent whitening agents include the sodium salts of: 4,4′-bis-(2-diethanolamino-4-anilino-s-triazin-6-ylamino) stilbene-2,2′-disulphonate; 4,4′-bis-(2,4-dianilino-s-triazin-6-ylamino) stilbene-2,2′-disulphonate; 4,4′-bis-(2-anilino-4(N-methyl-N-2-hydroxy-ethylamino)-s-triazin-6-ylamino) stilbene-2,2′-disulphonate, 4,4′-bis-(4-phenyl-2,1,3-triazol-2-yl)stilbene-2,2′-disulphonate; 4,4′-bis-(2-anilino-4(1-methyl-2-hydroxy-ethylamino)-s-triazin-6-ylamino) stilbene-2,2′-disulphonate and 2-(stilbyl-4″-naptho-1,2′:4,5)-1,2,3-trizole-2″-sulphonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4 anilino-s-triazin-6-ylamino) stilbene disulphonate. Tinopal CBS is the disodium salt of 2,2′-bis-(phenyl-styryl) disulphonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01, from 0.05, from about 0.1 or even from about 0.2 wt % to upper levels of 0.5 or even 0.75 wt %.

Soil Release Polymers:

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-Redeposition Agents:

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid. There are a number of detergent formulation forms such as layers (same or different phases), pouches, as well as forms for machine dosing unit.

Pouches can be configured as single or multicompartments. It can be of any form, shape and material which is suitable for hold the composition, e.g. without allowing the release of the composition from the pouch prior to water contact. The pouch is made from water soluble film which encloses an inner volume. Said inner volume can be devided into compartments of the pouch. Preferred films are polymeric materials preferably polymers which are formed into a film or sheet. Preferred polymers, copolymers or derivates therof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxyprpyl methyl cellulose (HPMC). Preferably the level of polymer in the film for example PVA is at least about 60%. Preferred average molecular weight will typically be about 20,000 to about 150,000. Films can also be of blend compositions comprising hydrolytically degradable and water soluble polymer blends such as polyactide and polyvinyl alcohol (known under the Trade reference M8630 as sold by Chris Craft In. Prod. Of Gary, Ind., US) plus plasticisers like glycerol, ethylene glycerol, Propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids. Ref: (US2009/0011970 A1).

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

The enzymes of the invention may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

The laundry soap bar may contain one or more additional enzymes, protease inhibitors such as peptide aldehydes (or hydrosulfite adduct or hemiacetal adduct), boric acid, borate, borax and/or phenylboronic acid derivatives such as 4-formylphenylboronic acid, one or more soaps or synthetic surfactants, polyols such as glycerine, pH controlling compounds such as fatty acids, citric acid, acetic acid and/or formic acid, and/or a salt of a monovalent cation and an organic anion wherein the monovalent cation may be for example Na+, K+ or NH₄⁺ and the organic anion may be for example formate, acetate, citrate or lactate such that the salt of a monovalent cation and an organic anion may be, for example, sodium formate.

The laundry soap bar may also contain complexing agents like EDTA and HEDP, perfumes and/or different type of fillers, surfactants e.g. anionic synthetic surfactants, builders, polymeric soil release agents, detergent chelators, stabilizing agents, fillers, dyes, colorants, dye transfer inhibitors, alkoxylated polycarbonates, suds suppressers, structurants, binders, leaching agents, bleaching activators, clay soil removal agents, anti-redeposition agents, polymeric dispersing agents, brighteners, fabric softeners, perfumes and/or other compounds known in the art.

The laundry soap bar may be processed in conventional laundry soap bar making equipment such as but not limited to: mixers, plodders, e.g a two stage vacuum plodder, extruders, cutters, logo-stampers, cooling tunnels and wrappers. The invention is not limited to preparing the laundry soap bars by any single method. The premix of the invention may be added to the soap at different stages of the process. For example, the premix containing a soap, an enzyme, optionally one or more additional enzymes, a protease inhibitor, and a salt of a monovalent cation and an organic anion may be prepared and and the mixture is then plodded. The enzyme and optional additional enzymes may be added at the same time as the protease inhibitor for example in liquid form. Besides the mixing step and the plodding step, the process may further comprise the steps of milling, extruding, cutting, stamping, cooling and/or wrapping.

Use to Degrade Xanthan Gum

Xanthan gum has been use as an ingredient in many consumer products including foods and cosmetics and has found use in the oil industry. Therefore the degradation of xanthan gum can result in improved cleaning processes, such as the easier removal of stains containing gums, such as xanthan gum, as well as the degradation of xanthan gum which is often used in the oil and drilling industry. Thus the present invention is directed to the use of endoglucanases of the invention or compositions thereof to degrade xanthan gum. The present invention is also directed to the use of xanthan lyases or compositions thereof to degrade xanthan gum. An embodiment is the use of endoglucanases of the invention together with xanthan lyases or compositions thereof to degrade xanthan gum. Degradation of xanthan gum can preferably be measured using the viscosity reduction assay (ViPr assay) as described in Example 5 or alternatively the reducing ends assay as described in Example 6.

In an embodiment, degradation of xanthan gum may be measured using the viscosity reduction assay as described herein on xanthan gum. A preferred embodiment is the use of xanthan gum (0.25% or 0.5%) in buffer or water wherein the drop in viscosity is measured after 5 minutes, 30 minutes, 1 hour, 1.5 hours, 2 hours, 2.5 hours, 3 hours, 3.5 hours or 4 hours. A more preferred embodiment is the use of xanthan gum (0.25%) in water wherein the drop in viscosity is measured after 3 hours.

The drop in viscosity for the degradation of xanthan gum is at least 200 Pa when using the viscosity reduction assay. The drop in viscosity for the degradation of xanthan gum is at least 250 Pa when using the viscosity reduction assay. The drop in viscosity for the degradation of xanthan gum is at least 300 Pa when using the viscosity reduction assay. The drop in viscosity for the degradation of xanthan gum is at least 350 Pa when using the viscosity reduction assay. The drop in viscosity for the degradation of xanthan gum is at least 400 Pa when using the viscosity reduction assay. The drop in viscosity for the degradation of xanthan gum is at least 450 Pa when using the viscosity reduction assay. The drop in viscosity for the degradation of xanthan gum is at least 500 Pa when using the viscosity reduction assay. The drop in viscosity for the degradation of xanthan gum is at least 550 Pa when using the viscosity reduction assay. The drop in viscosity for the degradation of xanthan gum is at least 600 Pa when using the viscosity reduction assay.

Xanthan degrading activity may alternatively be measured as reducing ends on xanthan gum pre-treated with xanthan lyase using the colorimetric assay developed by Lever (1972), Anal. Biochem. 47: 273-279, 1972. A preferred embodiment is the use of 0.1% xanthan gum pre-treated with xanthan lyase. Degradation of xanthan gum pre-treated with xanthan lyase may be determined by calculating difference between blank and sample wherein a difference of more than 0.1 mAU, more than 0.15 mAU, more than 0.2 mAU, more than 0.25 mAU more than 0.5 mAU, preferably more than 0.6 mAU, more preferably more than 0.7 mAU or even more preferably more than 0.8 mAU shows degradation of xanthan gum pre-treated with xanthan lyase.

Use in Detergents.

The present invention is directed to the use of endoglucanases of the invention or compositions thereof in cleaning processes such as the laundering of textiles and fabrics (e.g. household laundry washing and industrial laundry washing), as well as household and industrial hard surface cleaning, such as dish wash. The endoglucanases of the invention may be added to a detergent composition comprising of one or more detergent components.

An embodiment is the use of endoglucanases of the invention together with xanthan lyases or compositions thereof in cleaning processes such as the laundering of textiles and fabrics (e.g. household laundry washing and industrial laundry washing), as well as household and industrial hard surface cleaning, such as dish wash. The endoglucanases of the invention together with xanthan lyases may be added to a detergent composition comprising of one or more detergent components.

The polypeptides of the present invention may be added to and thus become a component of a detergent composition. The detergent composition of the present invention may be formulated, for example, as a hand or machine laundry detergent composition for both household and industrial laundry cleaning, including a laundry additive composition suitable for pre-treatment of stained fabrics and a rinse added fabric softener composition, or be formulated as a detergent composition for use in general household or industrial hard surface cleaning operations, or be formulated for hand or machine (both household and industrial) dishwashing operations. In a specific aspect, the present invention provides a detergent additive comprising a polypeptide of the present invention as described herein.

In an embodiment, the ΔInt enzyme value may be measured using the AMSA on xanthan gum with carbon black swatches as described in WO 2013/167581. A preferred embodiment is the use of xanthan gum with carbon black (DN31, DN31C or DN31D) swatches at 20° C. or at 40° C. A more preferred embodiment is the use of xanthan gum with carbon black (DN31C or DN31D) swatches at 40° C. An even more preferred embodiment is the use of xanthan gum with carbon black (DN31D) swatches at 40° C. The preferred enzyme concentration used for the endoglucanase having activity on xanthan gum pretreated with xanthan lyase, and for the xanthan lyase is 0.5 mg EP/L and 1.0 mg EP/L respectively.

The delta intensity value for xanthan gum with carbon black swatch is at least 3 units as determined by AMSA. The delta intensity value for xanthan gum with carbon black swatch is at least 3.5 units as determined by AMSA. The delta intensity value for xanthan gum with carbon black swatch is at least 4 units as determined by AMSA. The delta intensity value for xanthan gum with carbon black swatch is at least 4.5 units as determined by AMSA. The delta intensity value for xanthan gum with carbon black swatch is at least 5 units as determined by AMSA. The delta intensity value for xanthan gum with carbon black swatch is at least 5.5 units as determined by AMSA. The delta intensity value for xanthan gum with carbon black swatch is at least 6 units as determined by AMSA. The delta intensity value for xanthan gum with carbon black swatch is at least 7 units as determined by AMSA. The delta intensity value for xanthan gum with carbon black swatch is at least 8 units as determined by AMSA. The delta intensity value for xanthan gum with carbon black swatch is at least 9 units as determined by AMSA. The delta intensity value for xanthan gum with carbon black swatch is at least 10 units as determined by AMSA.

In an embodiment, the ΔRem enzyme value may be measured using the MiniLOM assay on xanthan gum with carbon black swatches as described in WO 2013/167581. A preferred embodiment is the use of xanthan gum with carbon black (DN31, DN31C or DN31D) swatches at 20° C. or at 40° C. A more preferred embodiment is the use of xanthan gum with carbon black (DN31C or DN31D) swatches at 40° C. An even more preferred embodiment is the use of xanthan gum with carbon black (DN31D) swatches at 40° C. The remission value is preferably measured at 460 nm. The preferred enzyme concentration used for the endoglucanase having activity on xanthan gum pretreated with xanthan lyase, and for the xanthan lyase is 0.5 mg EP/L and 1.0 mg EP/L respectively.

The ΔRem enzyme value for xanthan gum with carbon black swatch is at least 1.5 units as determined by MiniLOM. The ΔRem enzyme value for xanthan gum with carbon black swatch is at least 1.75 units as determined by MiniLOM. The ΔRem enzyme value for xanthan gum with carbon black swatch is at least 2 units as determined by MiniLOM. The ΔRem enzyme value for xanthan gum with carbon black swatch is at least 2.25 units as determined by MiniLOM. The ΔRem enzyme value for xanthan gum with carbon black swatch is at least 2.5 units as determined by MiniLOM. The ΔRem enzyme value for xanthan gum with carbon black swatch is at least 2.75 units as determined by MiniLOM. The ΔRem enzyme value for xanthan gum with carbon black swatch is at least 3 units as determined by MiniLOM. The ΔRem enzyme value for xanthan gum with carbon black swatch is at least 3.5 units as determined by MiniLOM. The ΔRem enzyme value for xanthan gum with carbon black swatch is at least 4 units as determined by MiniLOM. The ΔRem enzyme value for xanthan gum with carbon black swatch is at least 4.5 units as determined by MiniLOM. The ΔRem enzyme value for xanthan gum with carbon black swatch is at least 5 units as determined by MiniLOM.

The invention also relates to methods for degrading xanthan gum on the surface of a textile or hard surface, such as dish wash, comprising applying a composition comprising one or more endoglucanases of the invention to xanthan gum. The invention further relates to methods for degrading xanthan gum on the surface of a textile or hard surface, such as dish wash, comprising applying a composition comprising one or more xanthan lyases to xanthan gum. An embodiment is a method for degrading xanthan gum on the surface of a textile or hard surface, such as dish wash, comprising applying a composition comprising one or more endoglucanases of the invention together with one or more xanthan lyases to xanthan gum. An embodiment is the composition comprising one or more detergent components as described herein.

Use in the Fracturing of a Subterranean Formation (Oil and/or Gas Drilling)

Hydraulic fracturing is used to create subterranean fractures that extend from the borehole into rock formation in order to increase the rate at which fluids can be produced by the formation. Generally, a high viscosity fracturing fluid is pumped into the well at sufficient pressure to fracture the subterranean formation. In order to maintain the increased exposure to the formation, a solid proppant is added to the fracturing fluid which is carried into the fracture by the high pressure applied to the fluid. Once the high viscosity fracturing fluid has carried the proppant into the formation, breakers are used to reduce the fluid's viscosity which allows the proppant to settle into the fracture and thereby increase the exposure of the formation to the well. Breakers work by reducing the molecular weight of the polymers, thus ‘breaking’ or degrading the polymer. The fracture then becomes a high permeability conduit for fluids and gas to be produced back to the well. Such processes are further disclosed in U.S. Pat. Nos. 7,360,593, 5,806,597, 5,562,160, 5,201,370 and 5,067,566.

Thus the invention relates to the use of an endoglucanase of the invention as enzyme breakers. An embodiment of the invention is the use of an endoglucanase of the invention together with xanthan lyases as enzyme breakers.

Accordingly, the invention provides a method for breaking xanthan gum in a well bore comprising: (i) blending together a gellable fracturing fluid comprising aqueous fluid, one or more hydratable polymers, suitable cross-linking agents for cross-linking the hydratable polymer to form a polymer gel and one or more enzymes of the invention (i.e. the enzyme breaker); (ii) pumping the cross-linked polymer gel into the well bore under sufficient pressure to fracture the surrounding formation; and (iii) allowing the enzyme breaker to degrade the cross-linked polymer to reduce the viscosity of the fluid so that the fluid can be pumped from the formation back to the well surface. As such, the endoglucanases of the invention can be used to control the viscosity of fracturing fluids. Furthermore, one or more endoglucanases of the invention together with one or more xanthan lyases can be used to control the viscosity of fracturing fluids.

The enzyme breaker of the present invention may be an ingredient of a fracturing fluid or a breaker-crosslinker-polymer complex which further comprises a hydratable polymer and a crosslinking agent. The fracturing fluid or complex may be a gel or may be gellable. The complex is useful in a method for using the complex in a fracturing fluid to fracture a subterranean formation that surrounds a well bore by pumping the fluid to a desired location within the well bore under sufficient pressure to fracture the surrounding subterranean formation. The complex may be maintained in a substantially non-reactive state by maintaining specific conditions of pH and temperature, until a time at which the fluid is in place in the well bore and the desired fracture is completed. Once the fracture is completed, the specific conditions at which the complex is inactive are no longer maintained. When the conditions change sufficiently, the complex becomes active and the breaker begins to catalyze polymer degradation causing the fracturing fluid to become sufficiently fluid to be pumped from the subterranean formation to the well surface.

Method of Degrading Xanthan Gum Wherein the Xanthan Gum is Used in Fracturing of a Subterranean Formation Perpetrated by a Well Bore

When a well is drilled, reservoir drilling fluid (RDF) is circulated within the drilling equipment to cool down and clean the drill bit, remove the drill cuttings out of the well bore, reduce friction between the drill string and the sides of the borehole, and form a filtercake in order to prevent fluid leak off into the formation. The driving force for the formation of the filtercake is the higher wellbore pressure applied to maintain the borehole stability. This filtercake restricts the inflow of reservoir fluids into the wellbore during the drilling process and placement of the completion. If the filtercake damage that is created during the drilling process is not removed prior to or during completion of the well, a range of issues can arise when the well is put on production, i.e., completion equipment failures and impaired reservoir productivity.

Drilling fluid (mud), also called reservoir drilling fluid (RDF), can be synthetic/oil based or water based. To minimize invasion of the drilling fluid into the formation, both oil based and water based mud filtercakes typically contain a bridging or weighting agent, usually particles of calcium carbonate, barite or a mixture of the two, that bridge at the pore throats of the formation and thereby form a relatively low permeability filtercake. Both oil based and water based mud filtercakes also contain solids called cuttings that have been picked up during drilling, as opposed to the bridging/weighting agents that are added in the formulation of the drilling fluid. These solids can be quartz (sand), silts and/or shales, depending on the reservoir formation as well as the formations traversed by the drilling path to the reservoir. In addition, oil based drilling muds contain water droplets that become trapped in the pore space of the filtercake, while water based mud filtercakes contain polymers, such as starch and xanthan gum, and other inorganic salts.

The formation of a mud filtercake is often necessary for drilling, particularly in unconsolidated formations with wellbore stability problems and typically high permeabilities. The filtercake is then treated with various chemicals, such as chelates or acids to dissolve the calcite component; and/or enzymes or oxidizers to degrade the polymer component to recover permeability.

In one aspect, the invention provides a method for degrading xanthan gum wherein xanthan gum is used in fracturing of a subterranean formation perpetrated by a well bore by applying a composition comprising one of more enzymes of the invention. The method can include the steps of: (i) pumping a treatment fluid comprising one or more enzymes of the invention into the borehole in contact with the filtercake to be removed to establish a differential pressure between the treatment fluid and the formation adjacent the filtercake and (ii) evenly propagating treatment of the filtercake during the differential pressure period to delay breakthrough by the treatment fluid.

In one embodiment, the method can include establishing permeability through the treated filtercake between the formation and the borehole. In another embodiment, the filtercake can include drilling solids and clays, and may be formed from an aqueous drilling fluid. If desired, the treatment fluid for treating the aqueous drilling fluid filtercake can also include an oxidizer and/or a chelate, or it can be substantially free of chelate and oxidizer additives. In another example, the filtercake can be formed from an oil or invert emulsion drilling fluid. If desired, the treatment fluid for treating the oil or invert emulsion drilling fluid filtercake can also include a mutual solvent, a water-wetting agent or a combination thereof to disperse hydrophobic components in the filtercake.

In one embodiment, the treatment fluid comprises one or more endoglucanases of the invention. In another embodiment, the treatment fluid comprises one or more xanthan lyases. In a preferred embodiment, the treatment fluid comprises one or more endoglucanases and one or more xanthan lyases.

Method of Degrading Xanthan Gum Wherein the Xanthan Gum is a Component in Borehole Filtercake

In one aspect, the invention provides a method for cleaning borehole filtercake, comprising polymers, such as xanthan gum and drilling fluid solids once the filtercake has been pumped to the surface. Drilling mud is pumped from mud pits to the drill bit and then back out to the surface, carrying out amongst other things crushed or cut rock (cuttings) in the process. The cuttings are filtered out and the mud is returned to the mud pits where fines can settle and/or chemicals or enzymes (breakers) can be added.

The method for degrading xanthan gum wherein the xanthan gum is a component in borehole filtercake can include the steps of (i) treating the borehole filtercake with a treatment fluid comprising one or more enzymes of the invention and (ii) separating the solids from the fluids. In one embodiment, the treatment fluid comprises one or more endoglucanases of the invention. In another embodiment, the treatment fluid comprises one or more xanthan lyases. In a preferred embodiment, the treatment fluid comprises one or more endoglucanases of the invention and one or more xanthan lyases.

The borehole filtercake may be treated in mud pits with one or more enzymes of the invention and the drilling fluid can be re-circulated. Alternatively, once the filtercake has been treated with one or more enzymes of the invention, the solids and fluid are separated using solid-liquid separation processes, such as centrifugation.

Use in Processing of Cellulosic Material.

The endoglucanase activity of the polypeptide of the present invention may also be applied for degrading or converting a cellulosic material, comprising: treating the cellulosic material with an enzyme composition comprising the polypeptide of the present invention. In a preferred aspect, the method further comprises recovering the degraded or converted cellulosic material.

The present invention also relates to methods of producing a fermentation product, comprising: (a) saccharifying a cellulosic material with an enzyme composition in the presence of a polypeptide of the present invention; (b) fermenting the saccharified cellulosic material with one or more (several) fermenting microorganisms to produce the fermentation product; and (c) recovering the fermentation product from the fermentation.

The present invention also relates to methods of fermenting a cellulosic material, comprising: fermenting the cellulosic material with one or more (several) fermenting microorganisms, wherein the cellulosic material is saccharified with an enzyme composition in the presence of a polypeptide of the present invention. In a preferred aspect, the fermenting of the cellulosic material produces a fermentation product. In another preferred aspect, the method further comprises recovering the fermentation product from the fermentation.

The methods of the present invention can be used to saccharify a cellulosic material to fermentable sugars and convert the fermentable sugars to many useful substances, e.g., fuel, potable ethanol, and/or fermentation products (e.g., acids, alcohols, ketones, gases, and the like). The production of a desired fermentation product from cellulosic material typically involves pretreatment, enzymatic hydrolysis (saccharification), and fermentation.

The processing of cellulosic material according to the present invention can be accomplished using processes conventional in the art. Moreover, the methods of the present invention can be implemented using any conventional biomass processing apparatus configured to operate in accordance with the invention.

Hydrolysis (saccharification) and fermentation, separate or simultaneous, include, but are not limited to, separate hydrolysis and fermentation (SHF); simultaneous saccharification and fermentation (SSF); simultaneous saccharification and cofermentation (SSCF); hybrid hydrolysis and fermentation (HHF); separate hydrolysis and co-fermentation (SHCF); hybrid hydrolysis and co-fermentation (HHCF); and direct microbial conversion (DMC).

A conventional apparatus can include a fed-batch stirred reactor, a batch stirred reactor, a continuous flow stirred reactor with ultrafiltration, and/or a continuous plug-flow column reactor (Corazza et al., 2003, Optimal control in fed-batch reactor for the cellobiose hydrolysis, Acta Scientiarum. Technology 25: 33-38; Gusakov and Sinitsyn, 1985, Kinetics of the enzymatic hydrolysis of cellulose: 1. A mathematical model for a batch reactor process, Enz. Microb. Technol. 7: 346-352), an attrition reactor (Ryu and Lee, 1983, Bioconversion of waste cellulose by using an attrition bioreactor, Biotechnol. Bioeng. 25: 53-65), or a reactor with intensive stirring induced by an electromagnetic field (Gusakov et al., 1996, Enhancement of enzymatic cellulose hydrolysis using a novel type of bioreactor with intensive stirring induced by electromagnetic field, Appl. Biochem. Biotechnol. 56: 141-153). Additional reactor types include: fluidized bed, upflow blanket, immobilized, and extruder type reactors for hydrolysis and/or fermentation.

Pretreatment.

In practicing the methods of the present invention, any pretreatment process known in the art can be used to disrupt plant cell wall components of cellulosic material (Chandra et al., 2007, Substrate pretreatment: The key to effective enzymatic hydrolysis of lignocellulosics? Adv. Biochem. Engin./Biotechnol. 108: 67-93; Galbe and Zacchi, 2007, Pretreatment of lignocellulosic materials for efficient bioethanol production, Adv. Biochem. Engin./Biotechnol. 108: 41-65; Hendriks and Zeeman, 2009, Pretreatments to enhance the digestibility of lignocellulosic biomass, Bioresource Technol. 100: 10-18; Mosier et al., 2005, Features of promising technologies for pretreatment of lignocellulosic biomass, Bioresource Technol. 96: 673-686; Taherzadeh and Karimi, 2008, Pretreatment of lignocellulosic wastes to improve ethanol and biogas production: A review, Int. J. of Mol. Sci. 9: 1621-1651; Yang and Wyman, 2008, Pretreatment: the key to unlocking low-cost cellulosic ethanol, Biofuels Bioproducts and Biorefining-Biofpr. 2: 26-40).

The cellulosic material can also be subjected to particle size reduction, pre-soaking, wetting, washing, or conditioning prior to pretreatment using methods known in the art.

Conventional pretreatments include, but are not limited to, steam pretreatment (with or without explosion), dilute acid pretreatment, hot water pretreatment, alkaline pretreatment, lime pretreatment, wet oxidation, wet explosion, ammonia fiber explosion, organosolv pretreatment, and biological pretreatment. Additional pretreatments include ammonia percolation, ultrasound, electroporation, microwave, supercritical CO₂, supercritical H₂O, ozone, and gamma irradiation pretreatments.

The cellulosic material can be pretreated before hydrolysis and/or fermentation. Pretreatment is preferably performed prior to the hydrolysis. Alternatively, the pretreatment can be carried out simultaneously with enzyme hydrolysis to release fermentable sugars, such as glucose, xylose, and/or cellobiose. In most cases the pretreatment step itself results in some conversion of biomass to fermentable sugars (even in absence of enzymes).

Steam Pretreatment. In steam pretreatment, cellulosic material is heated to disrupt the plant cell wall components, including lignin, hemicellulose, and cellulose to make the cellulose and other fractions, e.g., hemicellulose, accessible to enzymes. Cellulosic material is passed to or through a reaction vessel where steam is injected to increase the temperature to the required temperature and pressure and is retained therein for the desired reaction time. Steam pretreatment is preferably done at 140-230° C., more preferably 160-200C, and most preferably 170-190° C., where the optimal temperature range depends on any addition of a chemical catalyst. Residence time for the steam pretreatment is preferably 1-15 minutes, more preferably 3-12 minutes, and most preferably 4-10 minutes, where the optimal residence time depends on temperature range and any addition of a chemical catalyst. Steam pretreatment allows for relatively high solids loadings, so that cellulosic material is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. Patent Application No. 20020164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

A catalyst such as H₂SO₄ or SO₂ (typically 0.3 to 3% w/w) is often added prior to steam pretreatment, which decreases the time and temperature, increases the recovery, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762).

Chemical Pretreatment: The term “chemical treatment” refers to any chemical pretreatment that promotes the separation and/or release of cellulose, hemicellulose, and/or lignin. Examples of suitable chemical pretreatment processes include, for example, dilute acid pretreatment, lime pretreatment, wet oxidation, ammonia fiber/freeze explosion (AFEX), ammonia percolation (APR), and organosolv pretreatments.

In dilute acid pretreatment, cellulosic material is mixed with dilute acid, typically H₂SO₄, and water to form a slurry, heated by steam to the desired temperature, and after a residence time flashed to atmospheric pressure. The dilute acid pretreatment can be performed with a number of reactor designs, e.g., plug-flow reactors, counter-current reactors, or continuous counter-current shrinking bed reactors (Duff and Murray, 1996, supra; Schell et al., 2004, Bioresource Technol. 91: 179-188; Lee et al., 1999, Adv. Biochem. Eng. Biotechnol. 65: 93-115).

Several methods of pretreatment under alkaline conditions can also be used. These alkaline pretreatments include, but are not limited to, lime pretreatment, wet oxidation, ammonia percolation (APR), and ammonia fiber/freeze explosion (AFEX).

Lime pretreatment is performed with calcium carbonate, sodium hydroxide, or ammonia at low temperatures of 85-150° C. and residence times from 1 hour to several days (Wyman et al., 2005, Bioresource Technol. 96: 1959-1966; Mosier et al., 2005, Bioresource Technol. 96: 673-686). WO 2006/110891, WO 2006/110899, WO 2006/110900, and WO 2006/110901 disclose pretreatment methods using ammonia.

Wet oxidation is a thermal pretreatment performed typically at 180-200° C. for 5-15 minutes with addition of an oxidative agent such as hydrogen peroxide or over-pressure of oxygen (Schmidt and Thomsen, 1998, Bioresource Technol. 64: 139-151; Palonen et al., 2004, Appl. Biochem. Biotechnol. 117: 1-17; Varga et al., 2004, Biotechnol. Bioeng. 88: 567-574; Martin et al., 2006, J. Chem. Technol. Biotechnol. 81: 1669-1677). The pretreatment is performed at preferably 1-40% dry matter, more preferably 2-30% dry matter, and most preferably 5-20% dry matter, and often the initial pH is increased by the addition of alkali such as sodium carbonate.

A modification of the wet oxidation pretreatment method, known as wet explosion (combination of wet oxidation and steam explosion), can handle dry matter up to 30%. In wet explosion, the oxidizing agent is introduced during pretreatment after a certain residence time. The pretreatment is then ended by flashing to atmospheric pressure (WO 2006/032282).

Ammonia fiber explosion (AFEX) involves treating cellulosic material with liquid or gaseous ammonia at moderate temperatures such as 90-100° C. and high pressure such as 17-20 bar for 5-10 minutes, where the dry matter content can be as high as 60% (Gollapalli et al., 2002, Appl. Biochem. Biotechnol. 98: 23-35; Chundawat et al., 2007, Biotechnol. Bioeng. 96: 219-231; Alizadeh et al., 2005, Appl. Biochem. Biotechnol. 121: 1133-1141; Teymouri et al., 2005, Bioresource Technol. 96: 2014-2018). AFEX pretreatment results in the depolymerization of cellulose and partial hydrolysis of hemicellulose. Lignin-carbohydrate complexes are cleaved.

Organosolv pretreatment delignifies cellulosic material by extraction using aqueous ethanol (40-60% ethanol) at 160-200° C. for 30-60 minutes (Pan et al., 2005, Biotechnol. Bioeng. 90: 473-481; Pan et al., 2006, Biotechnol. Bioeng. 94: 851-861; Kurabi et al., 2005, Appl. Biochem. Biotechnol. 121: 219-230). Sulphuric acid is usually added as a catalyst. In organosolv pretreatment, the majority of hemicellulose is removed.

Other examples of suitable pretreatment methods are described by Schell et al., 2003, Appl. Biochem. and Biotechnol. 105-108: 69-85, and Mosier et al., 2005, Bioresource Technology 96: 673-686, and U.S. Published Application No. 2002/0164730.

In one aspect, the chemical pretreatment is preferably carried out as an acid treatment, and more preferably as a continuous dilute and/or mild acid treatment. The acid is typically sulfuric acid, but other acids can also be used, such as acetic acid, citric acid, nitric acid, phosphoric acid, tartaric acid, succinic acid, hydrogen chloride, or mixtures thereof. Mild acid treatment is conducted in the pH range of preferably 1-5, more preferably 1-4, and most preferably 1-3. In one aspect, the acid concentration is in the range from preferably 0.01 to 20 wt % acid, more preferably 0.05 to 10 wt % acid, even more preferably 0.1 to 5 wt % acid, and most preferably 0.2 to 2.0 wt % acid. The acid is contacted with cellulosic material and held at a temperature in the range of preferably 160-220° C., and more preferably 165-195° C., for periods ranging from seconds to minutes to, e.g., 1 second to 60 minutes.

In another aspect, pretreatment is carried out as an ammonia fiber explosion step (AFEX pretreatment step).

In another aspect, pretreatment takes place in an aqueous slurry. In preferred aspects, cellulosic material is present during pretreatment in amounts preferably between 10-80 wt %, more preferably between 20-70 wt %, and most preferably between 30-60 wt %, such as around 50 wt %. The pretreated cellulosic material can be unwashed or washed using any method known in the art, e.g., washed with water.

Mechanical Pretreatment: The term “mechanical pretreatment” refers to various types of grinding or milling (e.g., dry milling, wet milling, or vibratory ball milling).

Physical Pretreatment: The term “physical pretreatment” refers to any pretreatment that promotes the separation and/or release of cellulose, hemicellulose, and/or lignin from cellulosic material. For example, physical pretreatment can involve irradiation (e.g., microwave irradiation), steaming/steam explosion, hydrothermolysis, and combinations thereof.

Physical pretreatment can involve high pressure and/or high temperature (steam explosion). In one aspect, high pressure means pressure in the range of preferably about 300 to about 600 psi, more preferably about 350 to about 550 psi, and most preferably about 400 to about 500 psi, such as around 450 psi. In another aspect, high temperature means temperatures in the range of about 100 to about 300° C., preferably about 140 to about 235° C. In a preferred aspect, mechanical pretreatment is performed in a batch-process, steam gun hydrolyzer system that uses high pressure and high temperature as defined above, e.g., a Sunds Hydrolyzer available from Sunds Defibrator AB, Sweden.

Combined Physical and Chemical Pretreatment: Cellulosic material can be pretreated both physically and chemically. For instance, the pretreatment step can involve dilute or mild acid treatment and high temperature and/or pressure treatment. The physical and chemical pretreatments can be carried out sequentially or simultaneously, as desired. A mechanical pretreatment can also be included.

Accordingly, in a preferred aspect, cellulosic material is subjected to mechanical, chemical, or physical pretreatment, or any combination thereof, to promote the separation and/or release of cellulose, hemicellulose, and/or lignin.

Biological Pretreatment: The term “biological pretreatment” refers to any biological pretreatment that promotes the separation and/or release of cellulose, hemicellulose, and/or lignin from cellulosic material. Biological pretreatment techniques can involve applying lignin-solubilizing microorganisms (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh and Singh, 1993, Physicochemical and biological treatments for enzymatic/microbial conversion of cellulosic biomass, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Fermentation of lignocellulosic hydrolysates for ethanol production, Enz. Microb. Tech. 18: 312-331; and Vallander and Eriksson, 1990, Production of ethanol from lignocellulosic materials: State of the art, Adv. Biochem. Eng./Biotechnol. 42: 63-95).

Saccharification.

In the hydrolysis step, also known as saccharification, the cellulosic material, e.g., pretreated, is hydrolyzed to break down cellulose and alternatively also hemicellulose to fermentable sugars, such as glucose, cellobiose, xylose, xylulose, arabinose, mannose, galactose, and/or soluble oligosaccharides. The hydrolysis is performed enzymatically by an enzyme composition in the presence of a polypeptide of the present invention. The composition can further comprise one or more (several) hemicellulolytic or xylan degrading enzymes. The enzymes of the compositions can also be added sequentially.

Enzymatic hydrolysis is preferably carried out in a suitable aqueous environment under conditions that can be readily determined by one skilled in the art. In a preferred aspect, hydrolysis is performed under conditions suitable for the activity of the enzyme(s), i.e., optimal for the enzyme(s). The hydrolysis can be carried out as a fed batch or continuous process where the pretreated cellulosic material (substrate) is fed gradually to, for example, an enzyme containing hydrolysis solution.

The saccharification is generally performed in stirred-tank reactors or fermentors under controlled pH, temperature, and mixing conditions. Suitable process time, temperature and pH conditions can readily be determined by one skilled in the art. For example, the saccharification can last up to 200 hours, but is typically performed for preferably about 12 to about 96 hours, more preferably about 16 to about 72 hours, and most preferably about 24 to about 48 hours. The temperature is in the range of preferably about 25° C. to about 70° C., more preferably about 30° C. to about 65° C., and more preferably about 40° C. to 60° C., in particular about 50° C. The pH is in the range of preferably about 3 to about 8, more preferably about 3.5 to about 7, and most preferably about 4 to about 6, in particular about pH 5. The dry solids content is in the range of preferably about 5 to about 50 wt %, more preferably about 10 to about 40 wt %, and most preferably about 20 to about 30 wt %.

The enzyme composition preferably comprises enzymes having cellulolytic activity and/or xylan degrading activity. In one aspect, the enzyme composition comprises one or more (several) cellulolytic enzymes. In another aspect, the enzyme composition comprises one or more (several) xylan degrading enzymes. In another aspect, the enzyme composition comprises one or more (several) cellulolytic enzymes and one or more (several) xylan degrading enzymes.

The one or more (several) cellulolytic enzymes are preferably selected from the group consisting of an endoglucanase, a cellobiohydrolase, and a beta-glucosidase. The one or more (several) xylan degrading enzymes are preferably selected from the group consisting of a xylanase, an acetyxylan esterase, a feruloyl esterase, an arabinofuranosidase, a xylosidase, and a glucuronidase.

In another aspect, the enzyme composition further or even further comprises a polypeptide having cellulolytic enhancing activity (see, for example, WO 2005/074647, WO 2005/074656, and WO 2007/089290). In another aspect, the enzyme composition may further or even further comprise one or more (several) additional enzyme activities to improve the degradation of the cellulose-containing material. Preferred additional enzymes are hemicellulases (e.g., alpha-D-glucuronidases, alpha-L-arabinofuranosidases, endo-mannanases, beta-mannosidases, alpha-galactosidases, endo-alpha-L-arabinanases, beta-galactosidases), carbohydrate-esterases (e.g., acetyl-xylan esterases, acetyl-mannan esterases, ferulic acid esterases, coumaric acid esterases, glucuronoyl esterases), pectinases, proteases, ligninolytic enzymes (e.g., laccases, manganese peroxidases, lignin peroxidases, H₂O₂-producing enzymes, oxidoreductases), expansins, swollenins, or mixtures thereof. In the methods of the present invention, the additional enzyme(s) can be added prior to or during fermentation, e.g., during saccharification or during or after propagation of the fermenting microorganism(s).

One or more (several) components of the enzyme composition may be wild-type proteins, recombinant proteins, or a combination of wild-type proteins and recombinant proteins. For example, one or more (several) components may be native proteins of a cell, which is used as a host cell to express recombinantly one or more (several) other components of the enzyme composition. One or more (several) components of the enzyme composition may be produced as monocomponents, which are then combined to form the enzyme composition. The enzyme composition may be a combination of multicomponent and monocomponent protein preparations.

The enzymes used in the methods of the present invention may be in any form suitable for use in the processes described herein, such as, for example, a crude fermentation broth with or without cells removed, a cell lysate with or without cellular debris, a semi-purified or purified enzyme preparation, or a host cell as a source of the enzymes. The enzyme composition may be a dry powder or granulate, a non-dusting granulate, a liquid, a stabilized liquid, or a stabilized protected enzyme. Liquid enzyme preparations may, for instance, be stabilized by adding stabilizers such as a sugar, a sugar alcohol or another polyol, and/or lactic acid or another organic acid according to established processes.

The Invention is Further Summarized in the Paragraphs Below:

1. An polypeptide having endoglucanase activity and/or having activity on xanthan gum pretreated with xanthan lyase, selected from the group consisting of:

(a) a polypeptide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40;

(b) a polypeptide encoded by a polynucleotide that hybridizes under medium stringency conditions with (i) the mature polypeptide coding sequence of any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, and SEQ ID NO: 39, (ii), or the full-length complement of (i);

(c) a polypeptide encoded by a polynucleotide having at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to the mature polypeptide coding sequence of any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, and SEQ ID NO: 39;

(d) a variant of the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 comprising a substitution, deletion, and/or insertion at one or more positions; and

(e) a fragment of the polypeptide of (a), (b), (c), or (d) that has endoglucanase activity and has activity on xanthan gum pretreated with xanthan lyase.

2. The polypeptide of paragraph 1, having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40.
3. The polypeptide of any of paragraphs 1 or 2, which is encoded by a polynucleotide that hybridizes under medium-high stringency conditions with (i) the mature polypeptide coding sequence of any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, and SEQ ID NO: 39, or (ii) the full-length complement of (i).
4. The polypeptide of any of paragraphs 1-3, which is encoded by a polynucleotide having at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide coding sequence of any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, and SEQ ID NO: 39.
5. The polypeptide of any of paragraphs 1-4, consisting of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 or the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40.
6. The polypeptide of any of paragraphs 1-4, comprising any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 or the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40.
7. The polypeptide of any of paragraphs 1-4, which is a variant of the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 comprising a substitution, deletion, and/or insertion at one or more positions, such as up to 10, e.g., 1, 2, 3, 4, 5, 6, 7, 8, 9, or 10 positions.
8. The polypeptide of paragraphs 1 to 7, which is a fragment of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40, wherein the fragment has endoglucanase activity and has activity on xanthan gum pretreated with xanthan lyase.
9. The polypeptide of any of paragraphs 1 to 8, wherein the mature polypeptide are any of the polypeptide shown in positions 1 to 813 in SEQ ID NO: 2, the polypeptide shown in positions 1 to 836 in SEQ ID NO: 4, the polypeptide shown in positions 1 to 949 in SEQ ID NO: 6, the polypeptide shown in positions 1 to 830 in SEQ ID NO: 8, the polypeptide shown in positions 1 to 866 in SEQ ID NO: 10, the polypeptide shown in positions 1 to 827 in SEQ ID NO: 12, the polypeptide shown in positions 1 to 903 in SEQ ID NO: 14, the polypeptide shown in positions 1 to 932 in SEQ ID NO: 16, the polypeptide shown in positions 1 to 920 in SEQ ID NO:18, the polypeptide shown in positions 1 to 844 in SEQ ID NO:20, the polypeptide shown in positions 1 to 849 in SEQ ID NO:22, the polypeptide shown in positions 1 to 903 in SEQ ID NO:24, the polypeptide shown in positions 1 to 894 in SEQ ID NO:26, the polypeptide shown in positions 1 to 894 in SEQ ID NO:28, the polypeptide shown in positions 1 to 955 in SEQ ID NO:30, the polypeptide shown in positions 1 to 894 in SEQ ID NO:32, the polypeptide shown in positions 1 to 893 in SEQ ID NO:34, the polypeptide shown in positions 1 to 894 in SEQ ID NO:36, the polypeptide shown in positions 1 to 894 in SEQ ID NO:38, and the polypeptide shown in positions 1 to 867 in SEQ ID NO:40.
10. An polynucleotide encoding the polypeptide of any of paragraphs 1-9.
11. A nucleic acid construct or expression vector comprising the polynucleotide of paragraph 10 operably linked to one or more control sequences that direct the production of the polypeptide in an expression host.
12. A recombinant host cell comprising the polynucleotide of paragraph 10 operably linked to one or more control sequences that direct the production of the polypeptide.
13. A method of producing the polypeptide of any of paragraphs 1-9, comprising cultivating a cell, which in its wild-type form produces the polypeptide, under conditions conducive for production of the polypeptide.
14. The method of paragraph 13, further comprising recovering the polypeptide.
15. A method of producing a polypeptide having endoglucanase activity and/or having activity on xanthan gum pretreated with xanthan lyase, comprising cultivating the host cell of paragraph 12 under conditions conducive for production of the polypeptide.
16. A transgenic plant, plant part or plant cell transformed with a polynucleotide encoding the polypeptide of any of paragraphs 1-9.
17. A method of producing a polypeptide having endoglucanase activity and/or having activity on xanthan gum pretreated with xanthan lyase, comprising cultivating the transgenic plant or plant cell of paragraph 16 under conditions conducive for production of the polypeptide.
18. The method of paragraph 17, further comprising recovering the polypeptide.
19. A whole broth formulation or cell culture composition comprising a polypeptide of any of paragraphs 1-9.
20. A composition comprising the polypeptide of any of paragraphs 1-9.
21. The composition of paragraph 20 further comprising a polypeptide having xanthan lyase activity.
22. The composition of any of paragraphs 20 or 21 being a detergent composition comprising one or more detergent components.
23. The composition of any of paragraphs 20-22, wherein the detergent components are selected from the group comprising of surfactants, builders, hydrotopes, bleaching systems, polymers, fabric hueing agents, adjunct materials, dispersants, dye transfer inhibiting agents, fluorescent whitening agents and soil release polymers, or any mixture thereof.
24. The composition of any of paragraphs 20-23, wherein the detergent composition is in form of a bar, a homogenous tablet, a tablet having two or more layers, a pouch having one or more compartments, a regular or compact powder, a granulate, a paste, a gel, or a regular, compact or concentrated liquid.
25. Use of a composition according to any of paragraphs 20-23 for degrading xanthan gum.
26. The use of paragraph 25 for controlling the viscosity of drilling fluids.
27. The use of paragraph 24 for washing or cleaning a textile and/or a hard surface such as dish wash.
28. The use of paragraph 24, wherein the detergent composition has an enzyme detergency benefit.
29. A method for degrading xanthan gum comprising applying a composition according to any of paragraphs 20-24 to xanthan gum.
30. The method of paragraph 29, wherein the xanthan gum is on the surface of a textile or hard surface, such as dish wash.
31. The method of paragraph 29, wherein the xanthan gum is used in fracturing of a subterranean formation penetrated by a well bore.
32. The method of paragraph 29, wherein the xanthan gum is a component in a borehole filtercake.
33. A method for degrading or converting a cellulosic material, comprising: treating the cellulosic material with the enzyme composition according to any of paragraphs 20-24 or in the presence of the polypeptide of any of paragraphs 1-9.
34. The method of paragraph 33, wherein the cellulosic material is pretreated.
35. The method of paragraph 32 or 33, wherein the enzyme composition comprises one or more enzymes selected from the group consisting of a cellulase, a polypeptide having cellulolytic enhancing activity, a hemicellulase, an esterase, a protease, a laccase, or a peroxidase.
36. The method of paragraph 35, wherein the cellulase is one or more enzymes selected from the group consisting of an endoglucanase, a cellobiohydrolase, and a beta-glucosidase.
37. The method of paragraph 35, wherein the hemicellulase is one or more enzymes selected from the group consisting of a xylanase, an acetyxylan esterase, a feruloyl esterase, an arabinofuranosidase, a xylosidase, and a glucuronidase.
38. The method of any of paragraphs 33-37, further comprising recovering the degraded cellulosic material.
39. The method of paragraph 38, wherein the degraded cellulosic material is a sugar, preferably selected from the group consisting of glucose, xylose, mannose, galactose, and arabinose.
40. A method for producing a fermentation product, comprising:

(a) saccharifying a cellulosic material with an enzyme composition in the presence of the polypeptide of any of paragraph 1-9;

(b) fermenting the saccharified cellulosic material with one or more fermenting microorganisms to produce the fermentation product; and

(c) recovering the fermentation product from the fermentation.

The present invention is further described by the following examples that should not be construed as limiting the scope of the invention.

### EXAMPLES

### Example 1: Search for and Identification of Putative Endo-Glucanase Genes

A Hidden Markov model (HMM) was created based on the endoglucanase disclosed in SEQ ID NO 2 of the patent application no. EP14170186.2 and closest homologs in UniProt release 2013_04 (Bairoch et al., 2005) and internal databases, by extracting the C-terminal part of each cellulase, creating a multiple alignment using MAFFT (Katoh, 2002), and constructing the HMM using HMMER3's hmmbuild (Eddy, 2011). A HMMER3's hmmsearch (Eddy, 2011) for the conserved domain found 334 homologs in publically available genomic and metagenomic assemblies available from DOE Joint Genome Institute, NCBI (Database resources of the National Center for Biotechnology Information. Nucleic Acids Res. 2009 January), and The European Nucleotide Archive (Leinonen, Nucleic Acids Res. 2011 January). Blast hits were sorted using the following parameters; coverage >50%, bit score >50 and E-value <1e-05.

### Example 2: Bioinformatics Analysis of the Endo-Glucanase Encoding Gene

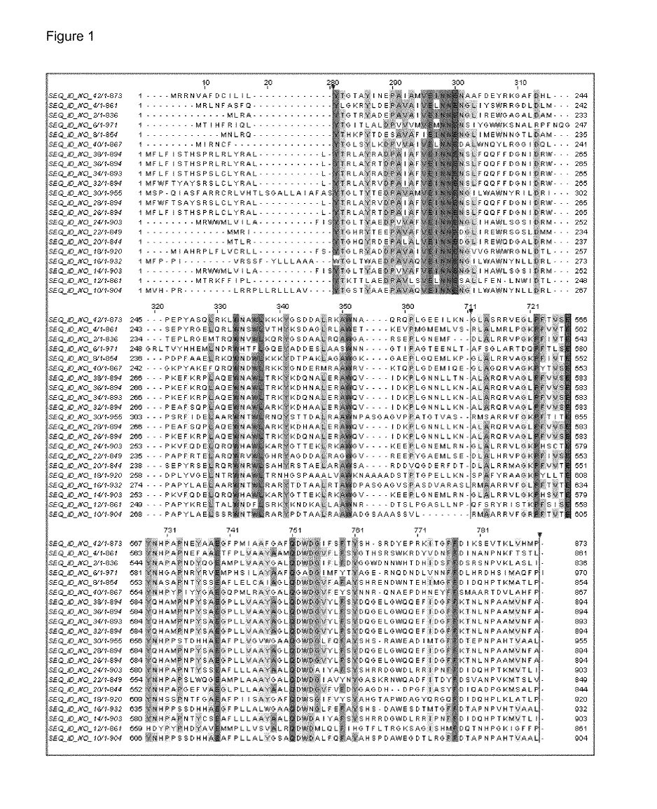

Twenty molecules selected by the procedure described in Example 1 were further classified by blast searches of the amino acid sequences (SEQ ID NO: 2, 4, 6, 8, 10, 12, 14, 16, 18, 20, 22, 24, 26, 28, 30, 32, 34, 36, 38, 40) against the Pfam database (M. Punta, P. C. Coggill, R. Y. Eberhardt, J. Mistry, J. Tate, C. Boursnell, N. Pang, K. Forslund, G. Ceric, J. Clements, A. Heger, L. Holm, E. L. L. Sonnhammer, S. R. Eddy, A. Bateman, R. D. Finn. Pfam: the protein families database. Nucleic Acids Research (2014) Database Issue 42:D222-D230) and by amino acid sequence alignment with the endoglucanase disclosed in SEQ ID NO 2 of the patent application no. EP14170186.2. In some instances, the blast searches against the Pfam database identified a very distantly related Pfam PF00150 domain with an % sequence identity and HMM score just above the noise cut-off defined by Pfam (% sequence cut-off of 20.7% and HMM score cut-off of 20.7). In most cases, the % sequence identity was below the noise cut-off. Moreover, the distantly related PF00150 domain was only partial, spanning approximately 150 residues out of 281 residues defined as the model length at the curation and model section available at the Pfam homepage (pfam.sanger.ac.uk). In addition, PF02018 domains or non-annotated regions equivalent in length to the PF02018 domain were identified in SEQ ID NO 2, 4, 6, 8, 10, 12, 14, 16, 18, 20, 22, 24, 26, 28, 30, 32, 34, 36, 38, 40. The postulated catalytic domain of PF00150 consists of two glutamates near the carboxy-terminal ends of β-strands four and seven, one acting as a proton donor and the other as the nucleophile (Jenkins J, Lo Leggio L, Harris G, and Pickersgill R. Beta-glucosidase, beta-galactosidase, family A cellulases, family F xylanases and two barley glycanases form a superfamily of enzymes with 8-fold beta/alpha architecture and with two conserved glutamates near the carboxy-terminal ends of beta-strands four and seven. FEBS Lett. 1995 Apr. 10; 362(3):281-5). Based on the amino acid sequence alignment, the putative glutamate proton donor and putative glutamate nucleophile were identified in all molecules (SEQ ID NO 2, 4, 6, 8, 10, 12, 14, 16, 18, 20, 22, 24, 26, 28, 30, 32, 34, 36, 38, 40), irrespective of the presence of a distantly related PF00150 domain (FIG. 1). The presence of the catalytic residues in highly conserved regions strongly indicates a close relationship between the molecules. The putative glutamate proton donor is located in the partial PF00150 domain and the putative nucleophile (E566) is located after the PF02018 domain (or in some instances a non-annotated region equivalent in length to the PF02018 domain). This suggests that the PF02018 domain has been introduced in between the glutamate catalytic residues, resulting in a novel domain architecture.

### Example 3: Cloning and Expression of Endo-Glucanases

The mature peptide encoding part of the endo-glucanase genes (positions 67 to 2508 in SEQ ID NO: 1, positions 76 to 2583 in SEQ ID NO: 3, positions 67 to 2913 in SEQ ID NO: 5 and positions 73 to 2562 in SEQ ID NO: 7) were obtained as synthetic genes. The mature peptide encoding part of the endo-glucanase genes were identified and inserted into E. coli. Expression plasmids containing the insert were purified from the E. coli transformants, and transformed into a Bacillus subtilis host cell. The transformed host cell was grown in liquid culture. Expression of the endo-glucanases was verified by SDS-page and activity testing.

### Example 4: Characterization of the Endo-Glucanases

A culture broth was centrifuged for 20 minutes at 20,000 g in a tabletop centrifuged and the supernatant was used directly for characterization of the endo-glucanases. An AZCL-HE-Cellulose (cross-linked and dyed cellulose) assay was used for detection of endo-glucancase activity. 1% AZCL-HE-Cellulose (from Megazyme) was suspended in 0.01% Triton X-100 by gentle stirring. 200 microliter of this suspension and 200 microliter assay buffer were mixed in an Eppendorf tube and placed on ice. 20 microliter endo-glucanase sample was added. The assay was initiated by transferring the Eppendorf tube to an Eppendorf thermomixer, which was set to the assay temperature. The tube was incubated for up to 60 min on the Eppendorf thermomixer at its highest shaking rate (1400 rpm). The incubation was stopped by transferring the tube back to the ice bath. The tube was then centrifuged in an icecold centrifuge for 2 min, 200 microliter supernatant was transferred to a microtitter plate and OD₅₉₀ was read. The Bacillus subtilis host strain was included in the assay. ΔOD₅₉₀=OD₅₉₀(enzyme)−OD₅₉₀(Bacillus subtilis host strain) was used a measure of endo-glucancase activity.

### Variable Assay Conditions

### Temperature: 30-80° C.

### Substrates: AZCL-HE-cellulose, AZCL-Pullulan, AZCL-xyloglucan and AZCL-curdlan.

Assay buffers: 100 mM succinic acid, 100 mM HEPES, 100 mM CHES, 100 mM CABS, 1 mM CaCl₂, 150 mM KCl, 0.01% Triton X-100 adjusted to pH 3-11.

The above endo-glucanase assay was used for obtaining the pH-activity profile, the temperature-activity profile as well as the substrate specificity profile.

TABLE 1
pH-activity profile (at 50° C.)

| | SEQ ID NO: 2 | SEQ ID NO: 8 |
|---|---|---|
| pH | ΔOD₅₉₀ | ΔOD₅₉₀ |
| 3 | 0 | 0 |
| 4 | 0 | 0 |
| 5 | 0.47 | 0.53 |
| 6 | 1.00 | 1.00 |
| 7 | 0.57 | 0.89 |
| 8 | 0.04 | 0.20 |
| 9 | 0 | 0.08 |
| 10 | 0 | 0 |
| 11 | 0 | 0 |

TABLE 2
Temperature-activity profile (at pH 6)

| | SEQ ID NO: 2 | SEQ ID NO: 8 |
|---|---|---|
| Temp (° C.) | ΔOD₅₉₀ | ΔOD₅₉₀ |
| 30 | 0.40 | 0.30 |
| 40 | 0.70 | 0.63 |
| 50 | 1.00 | 1.00 |
| 60 | 0.75 | 0.98 |
| 70 | 0.08 | 0.42 |
| 80 | 0.05 | 0.39 |

TABLE 3
Substrate specificity profile at pH 6, 50° C.

| | SEQ ID NO: 2 | SEQ ID NO: 8 |
|---|---|---|
| Substrate | ΔOD₅₉₀ | ΔOD₅₉₀ |
| HE-cellulose | 1.00 | 1.00 |
| Curdlan | 0.00 | 0.04 |
| Pullulan | 0.00 | 0.07 |
| Xyloglucan | 0.02 | 0.27 |

### Example 5: Xanthan Degrading Activity Viscosity Assay

Xanthan degrading activity of the polypeptide of the invention can assessed by measuring reduction in viscosity of a solution of xanthan gum or a solution of modified xanthan gum upon incubation with the endoglucanase. The viscosity measurements are performed using the viscosity pressure assay described in WO2011/107472.

The substrate modified xanthan gum (mXG) is xanthan gum (XG) treated with a xanthan lyase which removes the terminal pyruvated mannose, and was prepared using an adaption of the method described in Nankai, Hashimoto et al. 1999, Appl. Environ. Microbiol 65(6): 2520-2526: 2.5 g of xanthan gum (CP Kelco) is wet with 5 mL of 96% ethanol in a 2 L beaker. 500 mL of 100 mM ACES buffer pH 7.00 is added and the solution stirred at ambient temperature for 2 h. 250 μL of xanthan lyase (Megazyme product E-XANLB, Bacillus sp.) is added and the solution incubated for 20 h at 50° C. After hydrolysis 1400 mL of 96% ethanol is added to the 500 mL sample, under stirring. Precipitation occurs, and after approximately 5 min the ethanol is decanted thereby removing the pyruvated mannose residues. 500 mL of 96% ethanol is added again to the remaining solution, and decanted after any precipitation. The sample is dried on a Whatman filter GF/C on an evaporating funnel. The filters are dried at 50° C. for 20 h. The sample is collected, grinded and sieved through a 300 μM sieve.

The hydrolysis conditions are as follows: 50° C., 0.6% xanthan gum (XG) or 0.3% modified xanthan gum (mXG) in 50 mM HEPES buffer+0.01% triton X-100 pH 7.0. Enzyme is added upon thermal equilibration. The initial viscosity is measured, after thermal equilibration and prior to enzyme addition. Controls are the same with buffer added instead of enzyme.

The reduction in viscosity is a measure of enzyme activity. A significant drop in viscosity is observed when the endoglucanase and xanthan lyase are incubated together with xanthan gum, or when the endoglucanase is incubated alone with modified xanthan gum. This indicates that once the pyruvated mannose is removed, the substrate is now sterically available and is degraded by the endoglucanase.

Modified xanthan gum: The substrate modified xanthan gum (mXG) is xanthan gum (XG) treated with a xanthan lyase which removes the terminal pyruvated mannose, and was prepared using an adaption of the method described in Nankai, Hashimoto et al. 1999, Appl. Environ. Microbiol 65(6): 2520-2526: 2.5 g of xanthan gum (CP Kelco) is wet with 5 mL of 96% ethanol in a 2 L beaker. 500 mL of 100 mM ACES buffer pH 7.00 is added and the solution stirred at ambient temperature for 2 h. 250 μL of xanthan lyase (Megazyme product E-XANLB, Bacillus sp.) is added and the solution incubated for 20 h at 50° C. After hydrolysis 1400 mL of 96% ethanol is added to the 500 mL sample, under stirring. Precipitation occurs, and after approximately 5 min the ethanol is decanted thereby removing the pyruvated mannose residues. 500 mL of 96% ethanol is added again to the remaining solution, and decanted after any precipitation. The sample is dried on a Whatman filter GF/C on an evaporating funnel. The filters are dried at 50° C. for 20 h. The sample is collected, grinded and sieved through a 300 pM sieve.

### Example 6: Reducing Ends Assay

The endoglucanase activity is determined by reducing ends on xanthan gum pre-treated with xanthan lyase (mXG, prepared as in Example 5) using the colorimetric assay developed by Lever (1972), Anal. Biochem. 47: 273-279, 1972. Any reducing ends that are produced will react with PAHBAH generating an increase of colour which is proportional to the enzyme activity under the conditions used in the assay.

Xanthan lyase activity is determined by reducing ends as described in above except that 0.1% xanthan gum is used as substrate.

Materials and Chemicals:

0.1% Substrate: 6 ml (5 mg/ml) xanthan gum pre-treated with xanthan lyase in 24 ml Milli-Q water.

Activity buffer: 100 mM sodium acetate, 100 mM MES, 1 mM CaCl2, in 0.01% Triton X100, pH 7.

Ka-Na-tartrate/NaOH buffer: Dissolve Ka-Na-tartrate (50 g) and NaOH (20 g) in water to a total volume of 1 liter. Store at 4° C.

Stop solution: Dissolve PAHBAH (Sigma H-9882) in Ka-Na-tartrate/NaOH solution to a concentration of 15 mg/ml (e.g. dissolve 500 mg PAHBAH in 33 ml Ka-Na-tartrate/NaOH solution).

Sample Preparation:

The enzyme samples are diluted to 0.1 mg/ml in activity buffer in costarstrips using a BioMek liquid handler robot. 50 μl of substrate and 50 μl of each diluted sample is transferred to a 96-well PCR-MTP plate, 50 μl activity buffer is added to each sample and the solutions mixed. The sealed PCR-plate is incubated in a PCR machine at 37° C. for 15 min. then immediately cooled to 10° C. 75 μl of stop solution is added to each sample, the mixture is shaken, and 75 μl of each sample is discarded. The samples are incubated for 10 min. at 95° C., then 1 min. 10° C. 150 μl of each sample is transferred to a new 96-well PCR-MTP and the absorbance at 405 nm is measured.

The colourmetric response is proportional to the amount of reducing ends produced, and thus proportional to the amount of the endoglucanase present.

1. A composition comprising a polypeptide having endoglucanase activity and/or having activity on xanthan gum pretreated with xanthan lyase, selected from the group consisting of:
(a) a polypeptide having at least 60% sequence identity to the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40;
(b) a polypeptide encoded by a polynucleotide that hybridizes under medium stringency conditions with (i) the mature polypeptide coding sequence of any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, and SEQ ID NO: 39, (ii), or the full-length complement of (i);
(c) a polypeptide encoded by a polynucleotide having at least 60% sequence identity to the mature polypeptide coding sequence of SEQ ID NO: 1;
(d) a variant of the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 comprising a substitution, deletion, and/or insertion at one or more positions; and
(e) a fragment of the polypeptide of (a), (b), (c), or (d) that has endoglucanase activity and has activity on xanthan gum pretreated with xanthan lyase.

2. The composition of claim 1, wherein the polypeptide is a variant of the mature polypeptide of any of SEQ ID NO: 2, SEQ ID NO: 4, SEQ ID NO: 6, SEQ ID NO: 8, SEQ ID NO: 10, SEQ ID NO: 12; SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, and SEQ ID NO: 40 comprising a substitution, deletion, and/or insertion at one or more positions.

3. The composition of claim 1, wherein the mature polypeptide are any of the polypeptide shown in positions 1 to 813 in SEQ ID NO: 2, the polypeptide shown in positions 1 to 836 in SEQ ID NO: 4, the polypeptide shown in positions 1 to 949 in SEQ ID NO: 6, the polypeptide shown in positions 1 to 830 in SEQ ID NO: 8, the polypeptide shown in positions 1 to 866 in SEQ ID NO: 10, the polypeptide shown in positions 1 to 827 in SEQ ID NO: 12, the polypeptide shown in positions 1 to 903 in SEQ ID NO: 14, the polypeptide shown in positions 1 to 932 in SEQ ID NO: 16, the polypeptide shown in positions 1 to 920 in SEQ ID NO:18, the polypeptide shown in positions 1 to 844 in SEQ ID NO:20, the polypeptide shown in positions 1 to 849 in SEQ ID NO:22, the polypeptide shown in positions 1 to 903 in SEQ ID NO:24, the polypeptide shown in positions 1 to 894 in SEQ ID NO:26, the polypeptide shown in positions 1 to 894 in SEQ ID NO:28, the polypeptide shown in positions 1 to 955 in SEQ ID NO:30, the polypeptide shown in positions 1 to 894 in SEQ ID NO:32, the polypeptide shown in positions 1 to 893 in SEQ ID NO:34, the polypeptide shown in positions 1 to 894 in SEQ ID NO:36, the polypeptide shown in positions 1 to 894 in SEQ ID NO:38, and the polypeptide shown in positions 1 to 867 in SEQ ID NO:40.

4. The composition of claim 4 further comprising a polypeptide having xanthan lyase activity.

5. The composition of claim 1 being a detergent composition comprising one or more detergent components.

6. The composition of claim 1, wherein the detergent components are selected from the group comprising of surfactants, builders, hydrotopes, bleaching systems, polymers, fabric hueing agents, adjunct materials, dispersants, dye transfer inhibiting agents, fluorescent whitening agents and soil release polymers, or any mixture thereof.

7. The composition of claim 1, wherein the detergent composition is in form of a bar, a homogenous tablet, a tablet having two or more layers, a pouch having one or more compartments, a regular or compact powder, a granulate, a paste, a gel, or a regular, compact or concentrated liquid.

8. (canceled)

9. (canceled)

10. (canceled)

11. (canceled)

12. A method for degrading xanthan gum comprising applying a composition according to claim 1 to xanthan gum.

13. The method of claim 12, wherein the xanthan gum is on the surface of a textile or hard surface.

14. The method of claim 12, wherein the xanthan gum is used in fracturing of a subterranean formation penetrated by a well bore.

15. The method of claim 12, wherein the xanthan gum is a component in a borehole filtercake.
